SEQUENCE LISTING (235 sequences)

SEQ ID NO: 1 - DNA, 13 bp, Artificial Sequence
  Synthetic Oligonucleotide
ggccccggccccg

SEQ ID NO: 2 - DNA, 14 bp, Artificial Sequence
  Synthetic Oligonucleotide
ccccggccccggcc

SEQ ID NO: 3 - DNA, 15 bp, Artificial Sequence
  Synthetic Oligonucleotide
gccccggccccggcc

SEQ ID NO: 4 - DNA, 16 bp, Artificial Sequence
  Synthetic Oligonucleotide
ccccggccccggcccc

SEQ ID NO: 5 - DNA, 16 bp, Artificial Sequence
  Synthetic Oligonucleotide
cccggccccggccccg

SEQ ID NO: 6 - DNA, 17 bp, Artificial Sequence
  Synthetic Oligonucleotide
cggccccggccccggcc

SEQ ID NO: 7 - DNA, 18 bp, Artificial Sequence
  Synthetic Oligonucleotide
ccggccccggccccggcc

SEQ ID NO: 8 - DNA, 19 bp, Artificial Sequence
  Synthetic Oligonucleotide
cccggccccggccccggcc

SEQ ID NO: 9 - DNA, 20 bp, Artificial Sequence
  Synthetic Oligonucleotide
gccccggccccggccccggc

SEQ ID NO: 10 - DNA, 12 bp, Artificial Sequence
  Synthetic Oligonucleotide
ggccccggcccc

SEQ ID NO: 11 - DNA, 13 bp, Artificial Sequence
  Synthetic Oligonucleotide
ggccccggccccg

SEQ ID NO: 12 - DNA, 14 bp, Artificial Sequence
  Synthetic Oligonucleotide
ccccggccccggcc

SEQ ID NO: 13 - DNA, 15 bp, Artificial Sequence
  Synthetic Oligonucleotide
gccccggccccggcc

SEQ ID NO: 14 - DNA, 16 bp, Artificial Sequence
  Synthetic Oligonucleotide
ccccggccccggcccc

SEQ ID NO: 15 - DNA, 16 bp, Artificial Sequence
  Synthetic Oligonucleotide
cccggccccggccccg

SEQ ID NO: 16 - DNA, 17 bp, Artificial Sequence
  Synthetic Oligonucleotide
cggccccggccccggcc

SEQ ID NO: 17 - DNA, 18 bp, Artificial Sequence
  Synthetic Oligonucleotide
ccggccccggccccggcc

SEQ ID NO: 18 - DNA, 19 bp, Artificial Sequence
  Synthetic Oligonucleotide
cccggccccggccccggcc

SEQ ID NO: 19 - DNA, 20 bp, Artificial Sequence
  Synthetic Oligonucleotide
gccccggccccggccccggc

SEQ ID NO: 20 - DNA, 12 bp, Artificial Sequence
  Synthetic Oligonucleotide
ggccccggcccc

SEQ ID NO: 21 - DNA, 13 bp, Artificial Sequence
  Synthetic Oligonucleotide
ggccccggccccg

SEQ ID NO: 22 - DNA, 15 bp, Artificial Sequence
  Synthetic Oligonucleotide
gccccggccccggcc

SEQ ID NO: 23 - DNA, 15 bp, Artificial Sequence
  Synthetic Oligonucleotide
gccccggccccggcc

SEQ ID NO: 24 - DNA, 16 bp, Artificial Sequence
  Synthetic Oligonucleotide
cccggccccggccccg

SEQ ID NO: 25 - DNA, 16 bp, Artificial Sequence
  Synthetic Oligonucleotide
cccggccccggccccg

SEQ ID NO: 26 - DNA, 18 bp, Artificial Sequence
  Synthetic Oligonucleotide
ccggccccggccccggcc

SEQ ID NO: 27 - DNA, 18 bp, Artificial Sequence
  Synthetic Oligonucleotide
ccggccccggccccggcc

SEQ ID NO: 28 - DNA, 18 bp, Artificial Sequence
  Synthetic Oligonucleotide
ccggccccggccccggcc

SEQ ID NO: 29 - DNA, 20 bp, Artificial Sequence
  Synthetic Oligonucleotide
gccccggccccggccccggc

SEQ ID NO: 30 - DNA, 20 bp, Artificial Sequence
  Synthetic Oligonucleotide
gccccggccccggccccggc

SEQ ID NO: 31 - DNA, 16 bp, Artificial Sequence
  Synthetic Oligonucleotide
ggacaccgtaggttac

SEQ ID NO: 32 - DNA, 16 bp, Artificial Sequence
  Synthetic Oligonucleotide
ctagcgggacaccgta

SEQ ID NO: 33 - DNA, 16 bp, Artificial Sequence
  Synthetic Oligonucleotide
tctttcctagcgggac

SEQ ID NO: 34 - DNA, 16 bp, Artificial Sequence
  Synthetic Oligonucleotide
cacctctctttcctag

SEQ ID NO: 35 - DNA, 16 bp, Artificial Sequence
  Synthetic Oligonucleotide
ttgacgcacctctctt

SEQ ID NO: 36 - DNA, 16 bp, Artificial Sequence
  Synthetic Oligonucleotide
cgctgtttgacgcacc

SEQ ID NO: 37 - DNA, 16 bp, Artificial Sequence
  Synthetic Oligonucleotide
acttgtcgctgtttga

SEQ ID NO: 38 - DNA, 16 bp, Artificial Sequence
  Synthetic Oligonucleotide
ggcggaacttgtcgct

SEQ ID NO: 39 - DNA, 16 bp, Artificial Sequence
  Synthetic Oligonucleotide
tacgtgggcggaactt

SEQ ID NO: 40 - DNA, 16 bp, Artificial Sequence
  Synthetic Oligonucleotide
atcttttacgtgggcg

SEQ ID NO: 41 - DNA, 16 bp, Artificial Sequence
  Synthetic Oligonucleotide
agcgtcatcttttacg

SEQ ID NO: 42 - DNA, 16 bp, Artificial Sequence
  Synthetic Oligonucleotide
acaccaagcgtcatct

SEQ ID NO: 43 - DNA, 16 bp, Artificial Sequence
  Synthetic Oligonucleotide
gctgacacaccaagcg

SEQ ID NO: 44 - DNA, 16 bp, Artificial Sequence
  Synthetic Oligonucleotide
gggacggctgacacac

SEQ ID NO: 45 - DNA, 16 bp, Artificial Sequence
  Synthetic Oligonucleotide
gcagcagggacggctg

SEQ ID NO: 46 - DNA, 16 bp, Artificial Sequence
  Synthetic Oligonucleotide
aaccgggcagcaggga

SEQ ID NO: 47 - DNA, 16 bp, Artificial Sequence
  Synthetic Oligonucleotide
agaagcaaccgggcag

SEQ ID NO: 48 - DNA, 16 bp, Artificial Sequence
  Synthetic Oligonucleotide
caaaagagaagcaacc

SEQ ID NO: 49 - DNA, 16 bp, Artificial Sequence
  Synthetic Oligonucleotide
cgcccccaaaagagaa

SEQ ID NO: 50 - DNA, 16 bp, Artificial Sequence
  Synthetic Oligonucleotide
agaccccgcccccaaa

SEQ ID NO: 51 - DNA, 16 bp, Artificial Sequence
  Synthetic Oligonucleotide
cttgctagaccccgcc

SEQ ID NO: 52 - DNA, 16 bp, Artificial Sequence
  Synthetic Oligonucleotide
cctgctcttgctagac

SEQ ID NO: 53 - DNA, 16 bp, Artificial Sequence
  Synthetic Oligonucleotide
cccacacctgctcttg

SEQ ID NO: 54 - DNA, 16 bp, Artificial Sequence
  Synthetic Oligonucleotide
cctaaacccacacctg

SEQ ID NO: 55 - DNA, 16 bp, Artificial Sequence
  Synthetic Oligonucleotide
acacctcctaaaccca

SEQ ID NO: 56 - DNA, 16 bp, Artificial Sequence
  Synthetic Oligonucleotide
aaacacacacctccta

SEQ ID NO: 57 - DNA, 16 bp, Artificial Sequence
  Synthetic Oligonucleotide
agagggtgggaaaaac

SEQ ID NO: 58 - DNA, 16 bp, Artificial Sequence
  Synthetic Oligonucleotide
ggggagagagggtggg

SEQ ID NO: 59 - DNA, 16 bp, Artificial Sequence
  Synthetic Oligonucleotide
agtagtggggagagag

SEQ ID NO: 60 - DNA, 16 bp, Artificial Sequence
  Synthetic Oligonucleotide
agagcaagtagtgggg

SEQ ID NO: 61 - DNA, 16 bp, Artificial Sequence
  Synthetic Oligonucleotide
actgtgagagcaagta

SEQ ID NO: 62 - DNA, 16 bp, Artificial Sequence
  Synthetic Oligonucleotide
gcgagtactgtgagag

SEQ ID NO: 63 - DNA, 16 bp, Artificial Sequence
  Synthetic Oligonucleotide
ccctcagcgagtactg

SEQ ID NO: 64 - DNA, 16 bp, Artificial Sequence
  Synthetic Oligonucleotide
tgttcaccctcagcga

SEQ ID NO: 65 - DNA, 16 bp, Artificial Sequence
  Synthetic Oligonucleotide
ttttcttgttcaccct

SEQ ID NO: 66 - DNA, 16 bp, Artificial Sequence
  Synthetic Oligonucleotide
caggtcttttcttgtt

SEQ ID NO: 67 - DNA, 16 bp, Artificial Sequence
  Synthetic Oligonucleotide
ctttatcaggtctttt

SEQ ID NO: 68 - DNA, 16 bp, Artificial Sequence
  Synthetic Oligonucleotide
gttaatctttatcagg

SEQ ID NO: 69 - DNA, 16 bp, Artificial Sequence
  Synthetic Oligonucleotide
cttctggttaatcttt

SEQ ID NO: 70 - DNA, 16 bp, Artificial Sequence
  Synthetic Oligonucleotide
tgttttcttctggtta

SEQ ID NO: 71 - DNA, 16 bp, Artificial Sequence
  Synthetic Oligonucleotide
cctccttgttttcttc

SEQ ID NO: 72 - DNA, 16 bp, Artificial Sequence
  Synthetic Oligonucleotide
tgtttccctccttgtt

SEQ ID NO: 73 - DNA, 16 bp, Artificial Sequence
  Synthetic Oligonucleotide
tgcggttgtttccctc

SEQ ID NO: 74 - DNA, 16 bp, Artificial Sequence
  Synthetic Oligonucleotide
acaggctgcggttgtt

SEQ ID NO: 75 - DNA, 16 bp, Artificial Sequence
  Synthetic Oligonucleotide
cttgctacaggctgcg

SEQ ID NO: 76 - DNA, 16 bp, Artificial Sequence
  Synthetic Oligonucleotide
ccagagcttgctacag

SEQ ID NO: 77 - DNA, 16 bp, Artificial Sequence
  Synthetic Oligonucleotide
tgagttccagagcttg

SEQ ID NO: 78 - DNA, 16 bp, Artificial Sequence
  Synthetic Oligonucleotide
gactcctgagttccag

SEQ ID NO: 79 - DNA, 16 bp, Artificial Sequence
  Synthetic Oligonucleotide
gcgcgcgactcctgag

SEQ ID NO: 80 - DNA, 16 bp, Artificial Sequence
  Synthetic Oligonucleotide
cccctagcgcgcgact

SEQ ID NO: 81 - DNA, 12 bp, Artificial Sequence
  Synthetic Oligonucleotide
ccggccccggcc

SEQ ID NO: 82 - DNA, 12 bp, Artificial Sequence
  Synthetic Oligonucleotide
ccggccccggcc

SEQ ID NO: 83 - DNA, 12 bp, Artificial Sequence
  Synthetic Oligonucleotide
ccggccccggcc

SEQ ID NO: 84 - DNA, 12 bp, Artificial Sequence
  Synthetic Oligonucleotide
cccggccccggc

SEQ ID NO: 85 - DNA, 12 bp, Artificial Sequence
  Synthetic Oligonucleotide
cccggccccggc

SEQ ID NO: 86 - DNA, 12 bp, Artificial Sequence
  Synthetic Oligonucleotide
cccggccccggc

SEQ ID NO: 87 - DNA, 12 bp, Artificial Sequence
  Synthetic Oligonucleotide
ccccggccccgg

SEQ ID NO: 88 - DNA, 12 bp, Artificial Sequence
  Synthetic Oligonucleotide
ccccggccccgg

SEQ ID NO: 89 - DNA, 12 bp, Artificial Sequence
  Synthetic Oligonucleotide
ccccggccccgg

SEQ ID NO: 90 - DNA, 13 bp, Artificial Sequence
  Synthetic Oligonucleotide
cggccccggcccc

SEQ ID NO: 91 - DNA, 13 bp, Artificial Sequence
  Synthetic Oligonucleotide
cggccccggcccc

SEQ ID NO: 92 - DNA, 13 bp, Artificial Sequence
  Synthetic Oligonucleotide
cggccccggcccc

SEQ ID NO: 93 - DNA, 13 bp, Artificial Sequence
  Synthetic Oligonucleotide
ccggccccggccc

SEQ ID NO: 94 - DNA, 13 bp, Artificial Sequence
  Synthetic Oligonucleotide
ccggccccggccc

SEQ ID NO: 95 - DNA, 13 bp, Artificial Sequence
  Synthetic Oligonucleotide
ccggccccggccc

SEQ ID NO: 96 - DNA, 13 bp, Artificial Sequence
  Synthetic Oligonucleotide
gccccggccccgg

SEQ ID NO: 97 - DNA, 13 bp, Artificial Sequence
  Synthetic Oligonucleotide
gccccggccccgg

SEQ ID NO: 98 - DNA, 13 bp, Artificial Sequence
  Synthetic Oligonucleotide
gccccggccccgg

SEQ ID NO: 99 - DNA, 13 bp, Artificial Sequence
  Synthetic Oligonucleotide
ggccccggccccg

SEQ ID NO: 100 - DNA, 13 bp, Artificial Sequence
  Synthetic Oligonucleotide
ggccccggccccg

SEQ ID NO: 101 - DNA, 14 bp, Artificial Sequence
  Synthetic Oligonucleotide
ccggccccggcccc

SEQ ID NO: 102 - DNA, 14 bp, Artificial Sequence
  Synthetic Oligonucleotide
ccggccccggcccc

SEQ ID NO: 103 - DNA, 14 bp, Artificial Sequence
  Synthetic Oligonucleotide
ccggccccggcccc

SEQ ID NO: 104 - DNA, 14 bp, Artificial Sequence
  Synthetic Oligonucleotide
cccggccccggccc

SEQ ID NO: 105 - DNA, 14 bp, Artificial Sequence
  Synthetic Oligonucleotide
cccggccccggccc

SEQ ID NO: 106 - DNA, 14 bp, Artificial Sequence
  Synthetic Oligonucleotide
cccggccccggccc

SEQ ID NO: 107 - DNA, 14 bp, Artificial Sequence
  Synthetic Oligonucleotide
ggccccggccccgg

SEQ ID NO: 108 - DNA, 14 bp, Artificial Sequence
  Synthetic Oligonucleotide
ggccccggccccgg

SEQ ID NO: 109 - DNA, 14 bp, Artificial Sequence
  Synthetic Oligonucleotide
ggccccggccccgg

SEQ ID NO: 110 - DNA, 14 bp, Artificial Sequence
  Synthetic Oligonucleotide
cggccccggccccg

SEQ ID NO: 111 - DNA, 14 bp, Artificial Sequence
  Synthetic Oligonucleotide
cggccccggccccg

SEQ ID NO: 112 - DNA, 14 bp, Artificial Sequence
  Synthetic Oligonucleotide
cggccccggccccg

SEQ ID NO: 113 - DNA, 15 bp, Artificial Sequence
  Synthetic Oligonucleotide
cccggccccggcccc

SEQ ID NO: 114 - DNA, 15 bp, Artificial Sequence
  Synthetic Oligonucleotide
cccggccccggcccc

SEQ ID NO: 115 - DNA, 15 bp, Artificial Sequence
  Synthetic Oligonucleotide
cccggccccggcccc

SEQ ID NO: 116 - DNA, 15 bp, Artificial Sequence
  Synthetic Oligonucleotide
gccccggccccggcc

SEQ ID NO: 117 - DNA, 15 bp, Artificial Sequence
  Synthetic Oligonucleotide
cggccccggccccgg

SEQ ID NO: 118 - DNA, 15 bp, Artificial Sequence
  Synthetic Oligonucleotide
cggccccggccccgg

SEQ ID NO: 119 - DNA, 15 bp, Artificial Sequence
  Synthetic Oligonucleotide
cggccccggccccgg

SEQ ID NO: 120 - DNA, 15 bp, Artificial Sequence
  Synthetic Oligonucleotide
ccggccccggccccg

SEQ ID NO: 121 - DNA, 15 bp, Artificial Sequence
  Synthetic Oligonucleotide
ccggccccggccccg

SEQ ID NO: 122 - DNA, 15 bp, Artificial Sequence
  Synthetic Oligonucleotide
ccggccccggccccg

SEQ ID NO: 123 - DNA, 16 bp, Artificial Sequence
  Synthetic Oligonucleotide
ccccggccccggcccc

SEQ ID NO: 124 - DNA, 16 bp, Artificial Sequence
  Synthetic Oligonucleotide
ccccggccccggcccc

SEQ ID NO: 125 - DNA, 16 bp, Artificial Sequence
  Synthetic Oligonucleotide
ccccggccccggcccc

SEQ ID NO: 126 - DNA, 16 bp, Artificial Sequence
  Synthetic Oligonucleotide
cggccccggccccggc

SEQ ID NO: 127 - DNA, 16 bp, Artificial Sequence
  Synthetic Oligonucleotide
cggccccggccccggc

SEQ ID NO: 128 - DNA, 16 bp, Artificial Sequence
  Synthetic Oligonucleotide
cggccccggccccggc

SEQ ID NO: 129 - DNA, 16 bp, Artificial Sequence
  Synthetic Oligonucleotide
ccggccccggccccgg

SEQ ID NO: 130 - DNA, 16 bp, Artificial Sequence
  Synthetic Oligonucleotide
ccggccccggccccgg

SEQ ID NO: 131 - DNA, 16 bp, Artificial Sequence
  Synthetic Oligonucleotide
ccggccccggccccgg

SEQ ID NO: 132 - DNA, 16 bp, Artificial Sequence
  Synthetic Oligonucleotide
cccggccccggccccg

SEQ ID NO: 133 - DNA, 17 bp, Artificial Sequence
  Synthetic Oligonucleotide
cggccccggccccggcc

SEQ ID NO: 134 - DNA, 17 bp, Artificial Sequence
  Synthetic Oligonucleotide
cggccccggccccggcc

SEQ ID NO: 135 - DNA, 17 bp, Artificial Sequence
  Synthetic Oligonucleotide
cggccccggccccggcc

SEQ ID NO: 136 - DNA, 17 bp, Artificial Sequence
  Synthetic Oligonucleotide
ccggccccggccccggc

SEQ ID NO: 137 - DNA, 17 bp, Artificial Sequence
  Synthetic Oligonucleotide
ccggccccggccccggc

SEQ ID NO: 138 - DNA, 17 bp, Artificial Sequence
  Synthetic Oligonucleotide
ccggccccggccccggc

SEQ ID NO: 139 - DNA, 17 bp, Artificial Sequence
  Synthetic Oligonucleotide
cccggccccggccccgg

SEQ ID NO: 140 - DNA, 17 bp, Artificial Sequence
  Synthetic Oligonucleotide
cccggccccggccccgg

SEQ ID NO: 141 - DNA, 17 bp, Artificial Sequence
  Synthetic Oligonucleotide
cccggccccggccccgg

SEQ ID NO: 142 - DNA, 17 bp, Artificial Sequence
  Synthetic Oligonucleotide
ccccggccccggccccg

SEQ ID NO: 143 - DNA, 17 bp, Artificial Sequence
  Synthetic Oligonucleotide
ccccggccccggccccg

SEQ ID NO: 144 - DNA, 17 bp, Artificial Sequence
  Synthetic Oligonucleotide
ccccggccccggccccg

SEQ ID NO: 145 - DNA, 18 bp, Artificial Sequence
  Synthetic Oligonucleotide
cggccccggccccggccc

SEQ ID NO: 146 - DNA, 18 bp, Artificial Sequence
  Synthetic Oligonucleotide
cggccccggccccggccc

SEQ ID NO: 147 - DNA, 18 bp, Artificial Sequence
  Synthetic Oligonucleotide
cggccccggccccggccc

SEQ ID NO: 148 - DNA, 18 bp, Artificial Sequence
  Synthetic Oligonucleotide
cccggccccggccccggc

SEQ ID NO: 149 - DNA, 18 bp, Artificial Sequence
  Synthetic Oligonucleotide
cccggccccggccccggc

SEQ ID NO: 150 - DNA, 18 bp, Artificial Sequence
  Synthetic Oligonucleotide
cccggccccggccccggc

SEQ ID NO: 151 - DNA, 18 bp, Artificial Sequence
  Synthetic Oligonucleotide
ccccggccccggccccgg

SEQ ID NO: 152 - DNA, 18 bp, Artificial Sequence
  Synthetic Oligonucleotide
ccccggccccggccccgg

SEQ ID NO: 153 - DNA, 18 bp, Artificial Sequence
  Synthetic Oligonucleotide
ccccggccccggccccgg

SEQ ID NO: 154 - DNA, 19 bp, Artificial Sequence
  Synthetic Oligonucleotide
cggccccggccccggcccc

SEQ ID NO: 155 - DNA, 19 bp, Artificial Sequence
  Synthetic Oligonucleotide
cggccccggccccggcccc

SEQ ID NO: 156 - DNA, 19 bp, Artificial Sequence
  Synthetic Oligonucleotide
cggccccggccccggcccc

SEQ ID NO: 157 - DNA, 19 bp, Artificial Sequence
  Synthetic Oligonucleotide
ccggccccggccccggccc

SEQ ID NO: 158 - DNA, 19 bp, Artificial Sequence
  Synthetic Oligonucleotide
ccggccccggccccggccc

SEQ ID NO: 159 - DNA, 19 bp, Artificial Sequence
  Synthetic Oligonucleotide
ccggccccggccccggccc

SEQ ID NO: 160 - DNA, 19 bp, Artificial Sequence
  Synthetic Oligonucleotide
cccggccccggccccggcc

SEQ ID NO: 161 - DNA, 19 bp, Artificial Sequence
  Synthetic Oligonucleotide
cccggccccggccccggcc

SEQ ID NO: 162 - DNA, 19 bp, Artificial Sequence
  Synthetic Oligonucleotide
cccggccccggccccggcc

SEQ ID NO: 163 - DNA, 19 bp, Artificial Sequence
  Synthetic Oligonucleotide
ggccccggccccggccccg

SEQ ID NO: 164 - DNA, 19 bp, Artificial Sequence
  Synthetic Oligonucleotide
ggccccggccccggccccg

SEQ ID NO: 165 - DNA, 19 bp, Artificial Sequence
  Synthetic Oligonucleotide
ggccccggccccggccccg

SEQ ID NO: 166 - DNA, 20 bp, Artificial Sequence
  Synthetic Oligonucleotide
ccggccccggccccggcccc

SEQ ID NO: 167 - DNA, 20 bp, Artificial Sequence
  Synthetic Oligonucleotide
ccggccccggccccggcccc

SEQ ID NO: 168 - DNA, 20 bp, Artificial Sequence
  Synthetic Oligonucleotide
ccggccccggccccggcccc

SEQ ID NO: 169 - DNA, 20 bp, Artificial Sequence
  Synthetic Oligonucleotide
ccccggccccggccccggcc

SEQ ID NO: 170 - DNA, 20 bp, Artificial Sequence
  Synthetic Oligonucleotide
ccccggccccggccccggcc

SEQ ID NO: 171 - DNA, 20 bp, Artificial Sequence
  Synthetic Oligonucleotide
ccccggccccggccccggcc

SEQ ID NO: 172 - DNA, 20 bp, Artificial Sequence
  Synthetic Oligonucleotide
gccccggccccggccccggc

SEQ ID NO: 173 - DNA, 20 bp, Artificial Sequence
  Synthetic Oligonucleotide
cggccccggccccggccccg

SEQ ID NO: 174 - DNA, 20 bp, Artificial Sequence
  Synthetic Oligonucleotide
cggccccggccccggccccg

SEQ ID NO: 175 - DNA, 20 bp, Artificial Sequence
  Synthetic Oligonucleotide
cggccccggccccggccccg

SEQ ID NO: 176 - DNA, 16 bp, Artificial Sequence
  Synthetic Oligonucleotide
cgtcagtatgcgaatc

SEQ ID NO: 177 - DNA, 20 bp, Artificial Sequence
  Primer
caattccaccagtcgctaga

SEQ ID NO: 178 - DNA, 19 bp, Artificial Sequence
  Primer
ctgcggttgcggtgcctgc

SEQ ID NO: 179 - DNA, 20 bp, Artificial Sequence
  Primer
atcatttggggttttgatgg

SEQ ID NO: 180 - DNA, 20 bp, Artificial Sequence
  Primer
tcttggcaacagctggagat

SEQ ID NO: 181 - DNA, 20 bp, Artificial Sequence
  Primer
gttggaatgcagtgatgtcg

SEQ ID NO: 182 - DNA, 18 bp, Artificial Sequence
  Primer
aagaggcgcgggtagaag

SEQ ID NO: 183 - DNA, 19 bp, Artificial Sequence
  Primer
agtcgctagaggcgaaagc

SEQ ID NO: 184 - DNA, 20 bp, Artificial Sequence
  Primer
ccctctcatttctctgaccg

SEQ ID NO: 185 - DNA, 988 bp, Homo sapiens
tgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggcttt

SEQ ID NO: 186 - DNA, 988 bp, Homo sapiens
aaagcccgacacccagcttcggtcagagaaatgagagggaaagtaaaaatgcgtcgagctctgaggagagcccccgcttctacccgcgcctcttcccggcagccgaaccccaaacagccacccgccaggatgccgcctcctcactcacccactcgccaccgcctgcgcctccgccgccgcgggcgcaggcaccgcaaccgcagccccgccccgggcccgcccccgggcccgccccgaccacgccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggcccctagcgcgcgactcctgagttccagagcttgctacaggctgcggttgtttccctccttgttttcttctggttaatctttatcaggtcttttcttgttcaccctcagcgagtactgtgagagcaagtagtggggagagagggtgggaaaaacaaaaacacacacctcctaaacccacacctgctcttgctagaccccgcccccaaaagagaagcaaccgggcagcagggacggctgacacaccaagcgtcatcttttacgtgggcggaacttgtcgctgtttgacgcacctctctttcctagcgggacaccgtaggttacgtctgtctgttttctatgtgcgatgacgttttctcacgaggctagcgaaatggggcggggcaacttgtcctgttcttttatcttaagacccgctctggaggagcgttggcgcaatagcgtgtgcgaaccttaataggggaggctgctggatctggagaaagtgaagacgatttcgtggttttgaatggttttgtttgtgcttggtaggcagtgggcgctcaacaca

SEQ ID NO: 187 - DNA, 34322 bp, Homo sapiens
ttgtaagttctctgaggcatccccagaagctgatgctgccatgcttcctatacagcctgcagaaccatgagtcaattaaacctcttttctttgtaaattacccagtctcaagtatttctttatagcaatgcaagaatggactaatacagaaaattgttactgagaagaagggcattgctataaagatacctgaaaatgtagaagtgactttggaaccggctaacaggcagaagttgaaacattttagagggctcagaagaagacagaaagatgagagaaagtttggaactcgctaggaacttgttgagtggttgtaaccaaaatactgatagtgatatagacagtgaagtccaggctgaggaggtctcagatggaaatgagaaatttattgggaatgagtaaaggtcaggtttgctatgctttagcaaagagcttagctgcattgttcctctgttctagggatctgtgaaatcttagacttaagaatgatgatttagggtatctggcagaagaaatttctaagcagcagagtgttcaagaagtaacctagctgcttctaatagcctatgctcataggcatgagcacagaaatgacctgaaattggaacttacacttaaaagggaagcagagcataaaagtttgtaaattttgcagcctggccatgtggtagtaaagaaaagctcgttctcaggagaggaagtcaagcaggctgcataaatttgcataactaaaaggaaggcaagggctgataaccaaaacaatggggagaaagactcataggactaacaggcattttattttattttatttttattttattattattatactttaagttttagggtacatgtgcacaatgtgcaggttagttgcatatgtatacatgtgccatgctggtgtgctgcacccattaactcgtcatttagcattaggtatatctcctaatgctatccctcccccctcccccaccccacaacagtccccagagtgtgatgttccccttcctgtgtccatgtgttctcattgttcaattcccacctatgagtgagaacatgtggtgtttggttttttgaccttgcaatagtttactgagaatgacgatttccaatttcatccatgtccctacaaaggacatgaactcatcattttttatggctgcatagtattccatggtgtatatgtgccacattttcttaatccagtctatcactgttggacatttgggttggttccaagtctttgctattgtgaatagtgccacaataaacatagtgtgcatgtgtctttatagcagcaggatttatagtcctttgggtatatacccagtgatgggatggctgggtcaaatggtatttctagttctagatccctgaggaatcgccacactgacttccacaatggttgaactagtttacagtcccaccaacagtgtaaaagtgttcctaataggcattttaggctttcatggtggtccctctcatcacaggccccgaggcctaggaggactgaatcatttcctgggccaggcctagggcccctgctccctcttacagccttgggactctgctccctgaatcccagctgctcaaaggggcccaggtactgttacagtaggtagctaatcaggcatgagtggggtaagagagaagtccccaccacccaccaggaatgtcaggcaaccatcagatgatggtcaggcagttgtcatactgcctctctaaaatagtaattggttgcagccagcaccagggagaggcaacttctcaatagatagaaacacctgaaattggtaactgggcgcttccaataagatctcaggaactgagagagtgggcttaacatgcacattaagaggcaaaatggtgaagtatgacctttgggggcattccaccggaaaagggaagaaagcctcaggtaagcatgtatacaactccagtaaacacactgcacacgctcaccttccaagtgcaagcagggcaccatgcatgcggcaagctcacccttagggaaggaccaagggaaaggggcacaagatgtcagaagtaggccagtgtataagatcctaggttcaaggtcaaacagggcacttgacctccaaggtgcccacttgggcctcttccaaatgtactttcctttcattcctgttctaaagctttttaataaacttttactcctgctctgaaacttgtcgcagtctctttttctgccttatgcctcttggtcaaattctttcttctgaggaggcaagaattgaggttgctgcagacccacatggatttgcagctggtaactcagataactttcaccagtaagaatacagttcaggctgctgcttcacagggtgccaggcataagccttggtggcttccataagctgtgaagccggcgggcgcacataatgcaagagttgaggcttaagaagctctgcctagattttagaggatgtatgaaaaagcctggatgtccagacagaagcctgttactggggtggaatcctcatggagaacatctactagggaagcaaggagaagaaatgtggggttgcagcccccacagagagtcccctggggcactgcctagcagagctatgacaagacagccaccgtcctccagaccccagaatggtagatccaccaacaacttgcaccctgcagcctggaaaagctgcaagcactcaatgctagcccatgagagcagctgtgggagatgaaccctggaaaaccacaggggtggttctgcccaaggttttgggagcccactcattgcatcagtgttccctgggtgtgagtcaaaggagattatttcagagctttaacatttaatgactgcccggctggctttcagacttgcaatggggccctatagcctctttcttttggcagatttctccctttcggaatggcagtatctgcccaatgcctatacccccattgtatctttgaagcaattaccttgtttttgattttacaggttcataggtagaagggactagcttcgtctcaggtgagacttgggactttggacttttgaatgaatgctggatcgagttaagactttggggaactgttggtaaggcacgacagtattttgcaatatgagaaggacattagatttgggaggggccagagttggaataacatggtttggatctctgtccccacccaaatctcatgttcaactgtaatccccagtgttggaggttgggcctggtgggaggtgagtggattatggggtggcttctaatggttttgtacagtcccctcttggtactatatagtgagttctgacaagatctagttgtttaaacgtatgtagcacctcccatttctctcttcccccagttcctgccatgtgaagtctggggtctccctatgccttccatcatgattttaagttccctatggcctgcccagaagctgatccagccatgcttcttgtacagcctgcagaactgtgagccattaaacttttctttataaattacccagtttcagttatttctttatagcagtgtaagaatggactaacacaattattaacgctagtcctcatgttgtacattaaatctctagatgtattagacgtaactgcaactttgtaccctaccctacaattttctttccccccaagccccccaaccaagggtctactctgtttctataaattcagttgttttttaattccacgtataagtgaagtacaactcagtgtagaaacttggtaaatgctagctacttgttataagctgtcagtcaaaataaaaatacagagatgaatctctaaattaagtgatttatttgggaagaaagaattgcaattagggcatacatgtagatcagatggtcttcggtatatccacacaacaaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctatgcaaggttcattttgtaaatattggaaatggagtgataatacgtacttcaccagaggatttaatgagaccttatacgatccttagttcagtacctgactagtgcttcataaatgctttttcatccaatctgacaatctccagcttgtaattggggcatttagaacatttaatatgattattggcatggtaggttaaagctgtcatcttgctgttttctatttgttctttttgttttctccttacttttggatttttttattctactatgtcttttctattgtcttattaactatactctttgatttattttagtggttgttttagggttatacctctttctaatttaccagtttataaccagtttatatactacttgacatatagcttaagaaacttactgttgttgtctttttgctgttatggtcttaacgtttttatttctacaaacattataaactccacactttattgttttttaattttacttatacagtcaattatcttttaaagatatttaaatataaacattcaaaacaccccaattaaaagtcagagattgttaataccacatgatctcacttacacacagaattgaaaaacttggaactcatagaagcagagagtaaaaacatggttaccaggtgctggggagaggcggtgggctggggagatgttggtcaaagttagacaggaggaataagttcaagagatctattgtacaacttattcagttagataggaggaataagctaaagatcaagagatctattgtacaatgtgactataaccaacaacatatattgtacacttgaaaattgctaacagtatcttttaagtgttctctctacaaataaatatgtgaggtaatgtatatattaattaactgtagtcatttcacaatgtatacttatttcaaaacatcatattgtatgctataaatatatacaacttttatttttcaattttagaaatgtccttaaaaaatcagattttcagatca

SEQ ID NO: 188 - DNA, 1957 bp, Homo sapiens
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttataaaaaaaa

SEQ ID NO: 189 - DNA, 20 bp, Artificial Sequence
  Synthetic Oligonucleotide
ccccggccccggcccctagc

SEQ ID NO: 190 - DNA, 20 bp, Artificial Sequence
  Synthetic Oligonucleotide
gccccggccccggcccctag

SEQ ID NO: 191 - DNA, 20 bp, Artificial Sequence
  Synthetic Oligonucleotide
gccccggccccggcccctag

SEQ ID NO: 192 - DNA, 20 bp, Artificial Sequence
  Synthetic Oligonucleotide
ggccccggccccggccccta

SEQ ID NO: 193 - DNA, 18 bp, Artificial Sequence
  Synthetic Oligonucleotide
ccccggccccggccccta

SEQ ID NO: 194 - DNA, 16 bp, Artificial Sequence
  Synthetic Oligonucleotide
ccccggccccggcccc

SEQ ID NO: 195 - DNA, 16 bp, Artificial Sequence
  Synthetic Oligonucleotide
ccccggccccggcccc

SEQ ID NO: 196 - DNA, 20 bp, Artificial Sequence
  Synthetic Oligonucleotide
ccggccccggccccggcccc

SEQ ID NO: 197 - DNA, 19 bp, Artificial Sequence
  Synthetic Oligonucleotide
cggccccggccccggcccc

SEQ ID NO: 198 - DNA, 18 bp, Artificial Sequence
  Synthetic Oligonucleotide
ggccccggccccggcccc

SEQ ID NO: 199 - DNA, 17 bp, Artificial Sequence
  Synthetic Oligonucleotide
gccccggccccggcccc

SEQ ID NO: 200 - DNA, 16 bp, Artificial Sequence
  Synthetic Oligonucleotide
ccccggccccggcccc

SEQ ID NO: 201 - DNA, 20 bp, Artificial Sequence
  Synthetic Oligonucleotide
cccggccccggccccggccc

SEQ ID NO: 202 - DNA, 20 bp, Artificial Sequence
  Synthetic Oligonucleotide
cccggccccggccccggccc

SEQ ID NO: 203 - DNA, 19 bp, Artificial Sequence
  Synthetic Oligonucleotide
ccggccccggccccggccc

SEQ ID NO: 204 - DNA, 18 bp, Artificial Sequence
  Synthetic Oligonucleotide
cggccccggccccggccc

SEQ ID NO: 205 - DNA, 17 bp, Artificial Sequence
  Synthetic Oligonucleotide
ggccccggccccggccc

SEQ ID NO: 206 - DNA, 16 bp, Artificial Sequence
  Synthetic Oligonucleotide
gccccggccccggccc

SEQ ID NO: 207 - DNA, 20 bp, Artificial Sequence
  Synthetic Oligonucleotide
ccccggccccggccccggcc

SEQ ID NO: 208 - DNA, 20 bp, Artificial Sequence
  Synthetic Oligonucleotide
ccccggccccggccccggcc

SEQ ID NO: 209 - DNA, 19 bp, Artificial Sequence
  Synthetic Oligonucleotide
cccggccccggccccggcc

SEQ ID NO: 210 - DNA, 18 bp, Artificial Sequence
  Synthetic Oligonucleotide
ccggccccggccccggcc

SEQ ID NO: 211 - DNA, 17 bp, Artificial Sequence
  Synthetic Oligonucleotide
cggccccggccccggcc

SEQ ID NO: 212 - DNA, 16 bp, Artificial Sequence
  Synthetic Oligonucleotide
ggccccggccccggcc

SEQ ID NO: 213 - DNA, 20 bp, Artificial Sequence
  Synthetic Oligonucleotide
gccccggccccggccccggc

SEQ ID NO: 214 - DNA, 19 bp, Artificial Sequence
  Synthetic Oligonucleotide
ccccggccccggccccggc

SEQ ID NO: 215 - DNA, 18 bp, Artificial Sequence
  Synthetic Oligonucleotide
cccggccccggccccggc

SEQ ID NO: 216 - DNA, 17 bp, Artificial Sequence
  Synthetic Oligonucleotide
ccggccccggccccggc

SEQ ID NO: 217 - DNA, 16 bp, Artificial Sequence
  Synthetic Oligonucleotide
cggccccggccccggc

SEQ ID NO: 218 - DNA, 19 bp, Artificial Sequence
  Synthetic Oligonucleotide
gccccggccccggccccgg

SEQ ID NO: 219 - DNA, 18 bp, Artificial Sequence
  Synthetic Oligonucleotide
ccccggccccggccccgg

SEQ ID NO: 220 - DNA, 17 bp, Artificial Sequence
  Synthetic Oligonucleotide
cccggccccggccccgg

SEQ ID NO: 221 - DNA, 16 bp, Artificial Sequence
  Synthetic Oligonucleotide
ccggccccggccccgg

SEQ ID NO: 222 - DNA, 20 bp, Artificial Sequence
  Synthetic Oligonucleotide
acgccccggccccggccccg

SEQ ID NO: 223 - DNA, 18 bp, Artificial Sequence
  Synthetic Oligonucleotide
gccccggccccggccccg

SEQ ID NO: 224 - DNA, 17 bp, Artificial Sequence
  Synthetic Oligonucleotide
ccccggccccggccccg

SEQ ID NO: 225 - DNA, 20 bp, Artificial Sequence
  Synthetic Oligonucleotide
cacgccccggccccggcccc

SEQ ID NO: 226 - DNA, 20 bp, Artificial Sequence
  Synthetic Oligonucleotide
ccacgccccggccccggccc

SEQ ID NO: 227 - DNA, 18 bp, Artificial Sequence
  Synthetic Oligonucleotide
acgccccggccccggccc

SEQ ID NO: 228 - DNA, 20 bp, Artificial Sequence
  Synthetic Oligonucleotide
accacgccccggccccggcc

SEQ ID NO: 229 - DNA, 18 bp, Artificial Sequence
  Synthetic Oligonucleotide
cacgccccggccccggcc

SEQ ID NO: 230 - DNA, 17 bp, Artificial Sequence
  Synthetic Oligonucleotide
acgccccggccccggcc

SEQ ID NO: 231 - DNA, 20 bp, Artificial Sequence
  Synthetic Oligonucleotide
accacgccccggccccggcc

SEQ ID NO: 232 - DNA, 20 bp, Artificial Sequence
  Synthetic Oligonucleotide
ggccccggccccggccccgg

SEQ ID NO: 233 - DNA, 20 bp, Artificial Sequence
  Synthetic Oligonucleotide
cggccccggccccggccccg

SEQ ID NO: 234 - DNA, 19 bp, Artificial Sequence
  Synthetic Oligonucleotide
ggccccggccccggccccg

SEQ ID NO: 235 - DNA, 20 bp, Artificial Sequence
  Synthetic Oligonucleotide
gccttactctaggaccaaga

(19) United States
(12) Patent Application Publication
(10) Pub. No.: US 2016/0108396 A1
(43) Pub. Date: Apr. 21, 2016

(54) OLIGOMERS TARGETING HEXANUCLEOTIDE REPEAT EXPANSION IN HUMAN C9ORF72 GENE

(71) Applicant: Pfizer Inc., New York, NY (US)

(72) Inventors: Mads Aaboe Jensen, Farum (DK); Morten Lindow, København SV (DK)

(21) Appl. No.: 14/826,829

(22) Filed: Aug. 14, 2015

Related U.S. Application Data
(63) Continuation of application No. PCT/IB2015/056080, filed on Aug. 10, 2015.
(60) Provisional application No. 62/037,741, filed on Aug. 15, 2014.

(51) Int. Cl. C12N 15/113

(52) U.S. Cl. CPC C12N 15/113; C12N 2310/341; C12N 2310/3231; C12N 2310/11; C12N 2310/3341; C12N 2320/30; C12N 2310/322

(57) ABSTRACT

The disclosure relates to oligomers capable of targeting RNA expressed from the human C9ORF72 gene containing a pathogenic hexanucleotide repeat expansion. Such oligomers are useful for, among other things, reducing or eliminating C9ORF72 RNA and/or proteins translated therefrom, and treating or preventing diseases or disorders caused by, or associated with, hexanucleotide repeat expansion, including familial frontotemporal dementia (FTD) and familial amyotrophic lateral sclerosis (ALS).

### CROSS-REFERENCE TO RELATED APPLICATION

This application is a continuation application of International PCT Application No. PCT/IB2015/056080, filed 10 Aug. 2015, which claims priority to U.S. Provisional Application No. 62/037,741, filed 15 Aug. 2014, each of which is incorporated herein by reference in its entirety.

### REFERENCE TO SEQUENCE LISTING

The Sequence Listing submitted concurrently herewith under 37 CFR §1.821 in a computer readable form (CRF) via EFS-Web as file name PC072085A_Seq_ST25.txt is incorporated herein by reference. The electronic copy of the Sequence Listing was created on 30 Jul. 2015, with a file size of 93,135 bytes.

### BACKGROUND

Frontotemporal dementia (FTD) and amyotrophic lateral sclerosis (ALS) are severe neurological diseases with no effective treatment. FTD is a common cause of early-onset dementia and includes a group of disorders characterized clinically by abnormalities in behavior, language, and personality. ALS is typified by degeneration of motor neurons leading to muscle atrophy and paralysis. Due to their substantial clinical and pathological overlap, FTD and ALS have been proposed to constitute a disease spectrum. For example, frontal lobe impairment is seen in ALS and some FTD patients develop features of motor neuron disease. Recently, two independent groups identified an expansion of a hexanucleotide (GGGGCC) repeat present in a non-coding region of the Homo sapiens chromosome 9 open reading frame 72 (C9ORF72) gene (termed “hexanucleotide repeat expansion” or simply “repeat expansion,” as used interchangeably herein) as the most common genetic cause of ALS and FTD.

The C9ORF72 gene encodes long and short protein isoforms of a protein of uncertain function (Ling et al., Neuron 79: 416-438 (2011)). Although the mechanism by which C9ORF72 hexanucleotide repeat expansion causes the familial versions FTD and ALS remains to be elucidated, preliminary analysis suggests a variety of mechanisms may be at work. More recent studies found intracellular inclusions of aggregated dipeptide repeat proteins in the brains of patients with familial FTD or ALS, presumably generated by non-standard translation of the expanded GGGCC repeat. This observation suggests the hypothesis that the protein aggregates are neurotoxic and may in part explain the etiology of the diseases. Ash, et al., Neuron 77:639-646 (2013); Mori, et al., Science 339:1335-1338 (2013). Additionally, other groups found intranuclear foci of RNA transcripts containing the GGGGCC repeat in the brains of familial FTD or ALS, suggesting a toxic gain of function attributable to RNA. DeJesus-Hernandez, et al., Neuron 72:245-256 (2011); Donnelly, et al., Neuron 80:415-428 (2013).

In view of the evidence that the GGGGCC repeat expansion in the C9ORF72 gene can cause severe neurological disease in humans through mechanisms involving the accumulation of excessive concentrations of RNA and/or aggregated protein produced from the gene, there exists a need in the art for agents capable of reducing expression of RNA from the C9ORF72 gene comprising hexanucleotide repeats. The inventions described herein meet that need.

### SUMMARY OF DISCLOSURE

Disclosed are oligomers that are complementary to the C9ORF72 gene. In particular, the present disclosure provides gapmer compositions for targeting RNA containing a pathogenic number (e.g., at least 30 repeats) of hexanucleotide repeats transcribed from the C9ORF72 gene and methods of treating subjects with disorders associated with such hexanucleotide repeat expansion.
E1. In a first aspect of the invention, there is provided a gapmer comprising between 12 to 30 nucleosides, complementary to at least a 12 contiguous nucleobase portion of SEQ ID NO:187, wherein the gapmer further comprises in 5′ to 3′ order:
   (a) a 5′ flanking region consisting of 1 to 5 contiguously linked nucleosides, at least one of which is an LNA monomer,
   (b) a gap region consisting of contiguously linked deoxyribonucleosides; and
   (c) a 3′ flanking region consisting of 1 to 5 contiguously linked nucleosides, at least one of which is an LNA monomer.
   wherein the gapmer is capable of preferentially inhibiting expression of C9ORF72 sense transcript containing an expanded hexanucleotide repeat region when compared with inhibiting expression of a wild-type C9ORF72 sense transcript, and
   wherein the gapmer is further capable of inhibiting expression of C9ORF72 antisense transcript containing an expanded hexanucleotide repeat region.
      Described below are a number of embodiments (E) of this first aspect of the invention where, for convenience E1 is identical thereto.
E2. The gapmer of E1, wherein the expanded hexanucleotide region contains at least 20 hexanucleotide repeats.
E3. The gapmer of E1, wherein the expanded hexanucleotide region contains at least 30 hexanucleotide repeats.
E4. The gapmer of E1, wherein the expanded hexanucleotide region contains at least 50 hexanucleotide repeats.
E5. The gapmer of E1, wherein the expanded hexanucleotide region contains at least 75 hexanucleotide repeats.
E6. The gapmer of E1, wherein the expanded hexanucleotide region contains at least 100 hexanucleotide repeats.
E7. The gapmer of E1 or E2, wherein the IC₅₀ for inhibition of expression of the C9ORF72 sense transcript containing an expanded hexanucleotide repeat region is 50% or less of the 1050 for a wild-type C9ORF72.
E8. The gapmer of E1 or E2, wherein the IC₅₀ for inhibition of expression of the C9ORF72 sense transcript containing an expanded hexanucleotide repeat region is 40% or less of the 1050 for a wild-type C9ORF72.
E9. The gapmer of E1 or E2, wherein the IC₅₀ for inhibition of expression of the C9ORF72 sense transcript containing an expanded hexanucleotide repeat region is 30% or less of the 1050 for a wild-type C9ORF72.
E10. The gapmer of E1 or E2, wherein the IC₅₀ for inhibition of expression of the C9ORF72 sense transcript containing an expanded hexanucleotide repeat region is 20% or less of the 1050 for a wild-type C9ORF72.
E11. The gapmer of E1 or E2, wherein the IC₅₀ for inhibition of expression of the C9ORF72 sense transcript containing an expanded hexanucleotide repeat region is 10% or less of the 1050 for a wild-type C9ORF72.
E12. The gapmer of E1 or E2, wherein the IC₅₀ for inhibition of expression of the C9ORF72 sense transcript containing an expanded hexanucleotide repeat region is 5% or less of the 1050 for a wild-type C9ORF72.
E13. The gapmer of any of E1 to E12, which is capable of reducing the expression of the C9ORF72 sense transcript containing an expanded hexanucleotide repeat region in a cell with an 1050 of less than 10 micromolar without electroporation.
E14. The gapmer of any of any of E1 to E13, having a sequence that is identical to at least a 12 nucleobase portion of a nucleobase sequence selected from the group consisting of SEQ ID NO:1 to SEQ ID NO:175.
E15. The gapmer of E6, having a sequence that is identical to at least a 12 contiguous nucleobase portion of a nucleobase sequence selected from the group consisting of SEQ ID NOs: 9, 10, 11, 12, 13, 14, 16, 17, 19, 20, 21, 22, 23, 24, 26, 95, 98, 103, 106, 109, 119, 120, 123, 125, 126, 127, 134, 135, 142, 144, 145, 146, 147, 149, 151, 152, 153, 154, 155, 156, 157, 158, 159, 160, 161, 162, 163, 166, 167, 169, 170, 172, 173, 174, and 175.
E16. The gapmer of E6, having a sequence that is identical to at least a 12 contiguous nucleobase portion of a nucleobase sequence selected from the group consisting of SEQ ID NOs: 12, 13, 14, 20, 103, 106, 109, 149, 155, 158, 160 and 161.
E17. The gapmer of any of E1-E16, wherein the 1050 for the C9ORF72 sense transcript and 1050 for the C9ORF72 antisense transcript are less than 5 μM.
E18. The gapmer of E17, having a sequence selected from the group consisting of SEQ ID NOs: 9, 12, 13, 14, 16, 17, 19, 20, 21, 26, 103, 106, 109, 119, 126, 127, 135, 142, 144, 146, 147, 149, 154, 155, 156, 157, 158, 159, 160, 161, 162, 166, and 170.
E19. The gapmer of E17, having a sequence selected from the group consisting of SEQ ID NOs: 9, 13, 16, 17, 19, 106, 135, 142, 147, 154, 156, 157, 158, 159 and 162.
E20. The gapmer of any of the E1 to E19, wherein the gapmer sequence does not consist of any sequence selected from the group consisting of SEQ ID Nos:189-234.
E21. The gapmer of any of E1-E7, which is complementary to an at least 12 contiguous nucleobase portion between nucleobase 5159 and 11702 of SEQ ID NO:187, for example, the gapmer of E1-E7, which is complementary to an at least 12 contiguous nucleobase portion between nucleobases 5311-5359 of SEQ ID NO: 187.
E22. The gapmer of any of the E1 to E21 which is not complementary to an at least 5 contiguous nucleobase portion of SEQ ID NO:188.
E23. The gapmer of any of the E1 to E22, comprising at least one modified base.
E24. The gapmer of E10, wherein the modified base is 5-methylcytosine. In one particular embodiment, the gap region of the gapmer of any of E1-E8 comprises at least 5-methylcytosine.
E25. The gapmer of any of E1-E11, wherein all internucleoside linkages are phosphorothioate.
E26. The gapmer of any of E1-E12, wherein all of the nucleosides in the 5′ flanking region and 3′ flanking region are modified.
E27. The gapmer of any of E1-E13, wherein all modified nucleosides are LNA monomers.
E28. The gapmer of any of E1-E14, wherein all LNA monomers are beta-D-oxy-LNA monomers.
E29. The gapmer of any of E1-E15, wherein the nucleobase sequence of said gapmer is CCc cgg ccc cgg CC (SEQ ID NO:12), wherein the 5′ flanking region and 3′ flanking region are each respectively indicated by capital letters; and wherein the gap region is indicated by lower case letters.
E30. The gapmer of E16, wherein at least one cytosine is 5-methylcytosine.
E31. The gapmer of E16 or E17, wherein cytosines at positions 1, 2, 4, 10, 13, and 14 are 5-methylcytosine.
E32. The gapmer of any of E1 to E15, wherein the nucleobase sequence of said gapmer is GCc ccg gcc ccg gCC (SEQ ID NO:13), wherein the 5′ flanking region and 3′ flanking region are each respectively indicated by capital letters, and wherein the gap region is indicated by lower case letters.
E33. The gapmer of E19, wherein at least one cytosine is 5-methylcytosine.
E34. The gapmer of E19 or E20, wherein cytosines at positions 2, 5, 11, 14, and 15 are 5-methylcytosine.
E35. The gapmer of any of E1 to E15, wherein the nucleobase sequence of said gapmer is CCc cgg ccc cgg ccC C (SEQ ID NO:14), wherein the 5′ flanking region and 3′ flanking region are each respectively indicated by capital letters, and wherein the gap region is indicated by lower case letters.
E36. The gapmer of E22, wherein at least one cytosine is 5-methylcytosine.
E37. The gapmer of E22 or E23, wherein cytosines at positions 1, 2, 4, 10, 15, and 16 are 5-methylcytosine.
E38. The gapmer of any of E1 to E15, wherein the nucleobase sequence of said gapmer is Ggc ccc ggc ccC (SEQ ID NO:20), wherein the 5′ flanking region and 3′ flanking region are each respectively indicated by capital letters, and wherein the gap region is indicated by lower case letters.
E39. The gapmer of E25, wherein at least one cytosine is 5-methylcytosine.
E40. The gapmer of E25 or E26, wherein cytosines at positions 6 and 12 are 5-methylcytosine.
E41. The gapmer of any of E1 to E15, wherein the nucleobase sequence of said gapmer is CCg gcc ccg gcC CC (SEQ ID NO:103), wherein the 5′ flanking region and 3′ flanking region are each respectively indicated by capital letters, and wherein the gap region is indicated by lower case letters.
E42. The gapmer of E28, wherein at least one cytosine is 5-methylcytosine.
E43. The gapmer of E28 or E29, wherein cytosines at positions 1, 2, 8, 12, 13, and 14 are 5-methylcytosine.
E44. The gapmer of any of E1 to E43, wherein the nucleobase sequence of said gapmer is CCc ggc ccc ggC CC (SEQ ID NO:106), wherein the 5′ flanking region and 3′ flanking region are each respectively indicated by capital letters; and wherein the gap region is indicated by lower case letters.
E45. The gapmer of E31, wherein at least one cytosine is 5-methylcytosine.
E46. The gapmer of E31 or E32, wherein cytosines at positions 1, 2, 3, 9, 12, 13, and 14 are 5-methylcytosine.
E47. The gapmer of any of E1 to E15, wherein the nucleobase sequence of said gapmer is GGc ccc ggc ccC GG (SEQ ID NO:109), wherein the 5′ flanking region and 3′ flanking region are each respectively indicated by capital letters, and wherein the gap region is indicated by lower case letters.
E48. The gapmer of E34, wherein at least one cytosine is 5-methylcytosine.
E49. The gapmer of E34 or E35, wherein cytosines at positions 6 and 12 are 5-methylcytosine.
E50. The gapmer of any of E1 to E15, wherein the nucleobase sequence of said gapmer is CCc ggc ccc ggc ccC GGC (SEQ ID NO:149), wherein the 5′ flanking region and 3′ flanking region are each respectively indicated by capital letters, and wherein the gap region is indicated by lower case letters.
E51. The gapmer of E37, wherein at least one cytosine is 5-methylcytosine.
E52. The gapmer of E37 or E38, wherein cytosines at positions 1, 2, 3, 9, 15, and 18 are 5-methylcytosine.
E53. The gapmer of any of E1 to E15, wherein the nucleobase sequence of said gapmer is CGg ccc cgg ccc cgg CCC C (SEQ ID NO:155), wherein the 5′ flanking region and 3′ flanking region are each respectively indicated by capital letters, and wherein the gap region is indicated by lower case letters.
E54. The gapmer of E40, wherein at least one cytosine is 5-methylcytosine.
E55. The gapmer of E40 or E41, wherein cytosines at positions 1, 7, 13, 16, 17, 18, and 19 are 5-methylcytosine.
E56. The gapmer of any of E1 to E15, wherein the nucleobase sequence of said gapmer is CCg gcc ccg gcc ccg GCC C (SEQ ID NO:158), wherein the 5′ flanking region and 3′ flanking region are each respectively indicated by capital letters, and wherein the gap region is indicated by lower case letters.
E57. The gapmer of E43, wherein at least one cytosine is 5-methylcytosine.
E58. The gapmer of E43 or E44, wherein cytosines at positions 1, 2, 8, 14, 17, 18, and 19 are 5-methylcytosine.
E59. The gapmer of any of E1 to E15, wherein the nucleobase sequence of said gapmer is CCC ggc ccc ggc ccc gGC C (SEQ ID NO:160), wherein the 5′ flanking region and 3′ flanking region are each respectively indicated by capital letters, and wherein the gap region is indicated by lower case letters.
E60. The gapmer of E46, wherein at least one cytosine is 5-methylcytosine.
E61. The gapmer of E46 or E47, wherein cytosines at positions 1, 2, 3, 9, 15, 18, and 19 are 5-methylcytosine.
E62. The gapmer of any of E1 to E15, wherein the nucleobase sequence of said gapmer is CCc ggc ccc ggc ccc GGC C (SEQ ID NO:161), wherein the 5′ flanking region and 3′ flanking region are each respectively indicated by capital letters, and wherein the gap region is indicated by lower case letters.
E63. The gapmer of E49, wherein at least one cytosine is 5-methylcytosine.
E64. The gapmer of E49 or E50, wherein cytosines at positions 1, 2, 3, 9, 15, 18, and 19 are 5-methylcytosine.
E65. The gapmer of any of E1-E51, covalently conjugated to a nucleotide or non-nucleotide moiety selected from the group consisting of proteins, fatty acid chains, sugar residues, glycoproteins, polymers, or combinations thereof.
E66. A composition comprising the gapmer of any of E1 to E65 and a pharmaceutically acceptable carrier.
E67. A method of treating a disorder in a subject in need thereof wherein the subject is suffering from a disease or disorder mediated by or associated with repeat expansion, comprising administering to said subject a therapeutically effective amount of the gapmer of any of the preceding claims.
E68. The method of E54, wherein said disorder is a neurological disorder.
E69. The method of E55, wherein said neurological disorder is a neurodegenerative disease.
E70. The method of E56, wherein said neurodegenerative disease is associated with hexanucleotide repeat expansion in the C9ORF72 gene.
E71. The method of any of E54 to E57, wherein said neurodegenerative disease is selected from amyotrophic lateral sclerosis (ALS) and frontotemporal dementia (FTD).
E72. The method of any of E54 to E58, wherein the gapmer is administered into the central nervous system intrathecally or intraventricularly.
E73. Use of the gapmer of any of E1 to E52, or the composition of E53 in the manufacture of a medicament for the treatment of a neurological disorder.
E74. The use of E60, wherein said neurological disorder is a neurodegenerative disease.
E75. The use of E60, wherein said neurodegenerative disease is associated with hexanucleotide repeat expansion in the C9ORF72 gene.
E76. The use of E61 or E62, wherein said neurodegenerative disease is selected from amyotrophic lateral sclerosis (ALS) and frontotemporal dementia (FTD).
E77. The use of any of E60 to E63, wherein the gapmer is administered into the central nervous system intrathecally or intraventricularly.
E78. A method of inhibiting expression of a C9ORF72 transcript containing an expanded hexanucleotide repeat region in a cell, the method comprising contacting a cell containing a target nucleic acid with an effective amount of the gapmer of any of E1 to E52, or the composition of E53.
E79. The method of E65, wherein the contacting is in vitro or in vivo.
E80. The method of E65 or E66, wherein the C9ORF72 transcript is a C9ORF72 sense transcript.
E81. The method of any of E65 to E67, wherein the C9ORF72 transcript is a C9ORF72 antisense transcript.
E82. The method of any of E65 to E70, wherein the C9ORF72 transcript is a pre-mRNA.
E83. The method of any of E65-E69, wherein the gapmer hybridizes with a target sequence present in the expanded hexanucleotide region of the C9ORF72 sense transcript or C9ORF72 antisense transcript.
E84. A gapmer comprising 12 to 30 nucleosides, including a 5′ flanking region and a 3′ flanking region, each flanking region consisting of 1 to 5 nucleosides, all of which are LNA monomers, and a gap region between the flanking regions consisting of contiguously linked deoxyribonucleosides, wherein said gapmer hybridizes with no more than 6 mismatches to the hexanucleotide repeat region of either C9ORF72 sense transcript or C9ORF72 antisense transcript.
E85. The gapmer of E71, wherein said gapmer hybridizes with at least 1 mismatch to the hexanucleotide repeat region of a RNA transcript transcribed from either C9ORF72 sense transcript or C9ORF72 antisense transcript.
E86. The gapmer of E71 or E72, wherein said gapmer hybridizes with at least 3 mismatches to the hexanucleotide repeat region of a RNA transcript transcribed from either C9ORF72 sense transcript or C9ORF72 antisense transcript.
E87. The gapmer of any of E71 to E73, wherein said gapmer hybridizes with at least 5 mismatches to the hexanucleotide repeat region of a RNA transcript transcribed from either C9ORF72 sense transcript or C9ORF72 antisense transcript.
E88. The gapmer of any of E71 to E74, wherein all modified nucleosides are LNA monomers.
E89. The gapmer of any of E71 to E75, wherein all LNA monomers are beta-D-oxy-LNA monomers.
E90. The gapmer of any of E71 to E76, wherein all internucleoside linkages are phosphorothioate.
E91. The gapmer of any of E71 to E77, wherein said gapmer comprises at least one modified base.
E92. The gapmer of any of E71 to E78, wherein the modified base is 5-methylcytosine.
E93. Use of the gapmer of any of E1 to E52, or the composition of E53, for the treatment of a disorder in a subject in need thereof wherein the subject is suffering from a disease or disorder mediated by or associated with repeat expansion.
E94. A method of down-regulating the expression of C9ORF72 sense transcript containing an expanded hexanucleotide repeat region in a cell, tissue or organism comprising contacting or administering said cell, tissue or organism with an effective amount of one or more of the gapmers of any of E1 to E52, or the compositions of E53.
E95. A method of down-regulating the expression of C9ORF72 antisense transcript containing an expanded hexanucleotide repeat region in a cell, tissue or organism comprising contacting or administering said cell, tissue or organism with an effective amount of one or more of the gapmers of any of E1 to E52, or the compositions of E53.
E96. A method of simultaneously down-regulating the expression of C9ORF72 sense transcript containing an expanded hexanucleotide repeat region and C9ORF72 antisense transcript containing an expanded hexanucleotide repeat region in a cell, tissue or organism comprising contacting or administering said cell, tissue or organism with an effective amount of one or more of the gapmers of any of E1 to E52, or the compositions of E53.

Accordingly, the present disclosure provides oligomers comprising between 12 to 30 linked nucleosides, at least a 12 nucleobase portion of which is present within a nucleobase sequence selected from the group consisting of SEQ ID NO:1 to SEQ ID NO:176. In other embodiments, oligomers are gapmers which, in some embodiments, comprise (in 5′ to 3′ order), a 5′ flanking region consisting of contiguously linked nucleosides, at least one of which is modified, a gap region consisting of contiguously linked deoxyribonucleosides, and a 3′ flanking region consisting of contiguously linked nucleosides, at least one of which is modified. In some of these embodiments, the 5′ flanking region and 3′ flanking region each consist of 1 to 5 nucleosides, all of which are modified. In related embodiments, all the modified nucleosides are LNA monomers, including sometimes beta-D-oxy-LNA monomers. In some embodiments, all the internucleoside linkages are phosphorothioate. Bases in gapmers can also be modified, for example, by adding a methyl group to one or more cytosines to form 5-methylcytosine.

Specific examples of oligomers of the disclosure include those having the following nucleobase sequences: CCc cgg ccc cgg CC (SEQ ID NO:12), GCc ccg gcc ccg gCC (SEQ ID NO:13), CCc cgg ccc cgg ccC C (SEQ ID NO:14), Ggc ccc ggc ccC (SEQ ID NO:20), CCg gcc ccg gcC CC (SEQ ID NO:103), CCc ggc ccc ggC CC (SEQ ID NO:106), GGc ccc ggc ccC GG (SEQ ID NO:109), CCc ggc ccc ggc ccC GGC (SEQ ID NO:149), CGg ccc cgg ccc cgg CCC C (SEQ ID NO:155), CCg gcc ccg gcc ccg GCC C (SEQ ID NO:158), CCC ggc ccc ggc ccc gGC C (SEQ ID NO:160), and CCc ggc ccc ggc ccc GGC C (SEQ ID NO:161). In these exemplary gapmers, the 5′ and 3′ flanking regions contain LNA monomers, such as LNA nucleosides, indicated by capital letters (e.g., A, T, G, C), whereas the gap regions contain DNA monomers, such as DNA nucleosides, indicated by lower case letters (e.g, a, t, g, c). All the linkages forming the backbone of the exemplary gapmers are phosphorothioate. In related embodiments, at least one base in each of the exemplary gapmers is modified. For example, cytosines can be modified by the addition of a methyl group to form 5-methylcytosine. In related embodiments of the exemplary gapmers, all cytosines in the 5′ and 3′ flanking regions are 5-methylcytosine, and in the DNA gap region, any cytosine immediately preceding a guanine (e.g., “cg” or “cG”) is 5-methylcytosine, whereas all other cytosines in the gap region remain unmodified.

In certain embodiments, the gapmers of the present invention do not consist of sequences selected from the group consisting of SEQ ID NOs: 189-234.

Gapmers of the disclosure can be included in compositions also including pharmaceutically acceptable carriers or excipients. Gapmers can also be covalently conjugated to a non-polynucleotide moiety.

Gapmers of the disclosure can usefully be employed in methods of preventing or treating disorders, such as neurological disorders in subjects, such as human subjects, by administering to a subject in need of treatment a therapeutically effective amount of a gapmer. In some embodiments, the neurological disorder is a neurodegenerative disease, including neurodegenerative diseases associated with hexanucleotide repeat expansion in the C9ORF72 gene. Specific examples of such neurodegenerative diseases include amyotrophic lateral sclerosis (ALS) and frontotemporal dementia (FTD). Gapmers, particularly when included in compositions comprising a pharmaceutically acceptable carrier, can be administered into the central nervous system of a subject intrathecally or intraventricularly. Gapmers of the disclosure can additionally be used in the manufacture of a medicament for the treatment of a neurological disorder, such as a neurodegenerative disease, including neurodegenerative diseases associated with hexanucleotide repeat expansion in the C9ORF72 gene, for example, ALS and FTD.

Also provided is a method of reducing the amount of RNA transcribed from the C9ORF72 gene in a cell by contacting the cell in vitro or in vivo with an effective amount of a gapmer of the disclosure. In some embodiments, the RNA is transcribed from the minus strand, such as pre-mRNA or mRNA, and in other embodiments, the RNA is transcribed from the plus strand. In some other embodiments, the gapmer hybridizes with a target sequence present in the hexanucleotide region of RNA transcribed from both the minus and plus strands of the C9ORF72 gene and is effective to reduce the amount of one or the other, or both types of RNA so transcribed.

The disclosure further provides gapmers comprising 12 to 30 linked nucleosides, including a 5′ flanking region and a 3′ flanking region, each independently consisting of 1 to 5 nucleosides, all of which are modified, and a gap region consisting of contiguously linked deoxynucleosides positioned between the flanking regions, wherein the gapmer hybridizes with no more than 4 mismatches to the hexanucleotide repeat region of RNA transcripts transcribed from both the minus and plus strands of the C9ORF72 gene. In other embodiments, fewer mismatches are allowed, for example, 3 or fewer, 2 or fewer, or not more than 1. According to yet other embodiments, all modified nucleosides in the 5′ and/or 3′ flanking regions are LNA monomers, for example, beta-D-oxy-LNA monomers. In some other embodiments, all internucleoside linkages are phosphorothioate. In yet other embodiments, at least one base of the gapmer is modified, for example, by the addition of a methyl group, cytosine can be modified to form 5-methylcytosine.

### BRIEF DESCRIPTION OF THE FIGURES

FIG. 1. Graph reports data demonstrating effect on C9ORF72 pre-mRNA levels in human ND40063 cells of different doses of oligomer numbers 176, 13, 146, and 156 as disclosed in Table 4.

FIG. 2. Graph reports data demonstrating effect on levels of sense and antisense RNA produced from C9ORF72 in human ND40063 cells of different doses of oligomer numbers 61, 9, 154, 156, and 158 as disclosed in Table 4.

FIG. 3. Graph reports data demonstrating effect on C9ORF72 pre-mRNA levels in HEK-293 cells and human ND40063 cells of different doses of oligomer numbers 127, 144, 145, 146, and 147 as disclosed in Table 4.

FIG. 4. Nucleic acid sequence of the plus (SEQ ID NO:185) and minus (SEQ ID NO:186) strands of an exemplary human C9ORF72 gene containing 33 repeats of the hexanucleotide sequence GGGGCC. The sequence shown is based on that of NCBI reference sequence NG_031977.1, which contains three repeats of the hexanucleotide sequence and one partial repeat. Exon 1 is identified by bold underline font. The hexanucleotide repeat section is identified by bold italic font. Certain oligomers of the disclosure, including for example those identified by SEQ ID NOs 31 to 80, target sequences within the region encompassing exon 1 and the sequence up to the beginning of the repeat region. Other oligomers of the disclosure, including for example those identified by SEQ ID NOs: 1 to 30 and SEQ ID NOs: 81 to 176, target sequences within the hexanucleotide repeat region.

FIG. 5. Graph reports data comparing effect on C9ORF72 pre-mRNA levels in ND40063 cells of different doses of oligomer number 109 with 2′-MOE containing oligomers disclosed in Table 5.

### DETAILED DESCRIPTION

### Oligomers for Modulating Expression of the C9ORF72 Gene

Disclosed herein are compounds, compositions and methods for modulating the expression of C9ORF72. In particular, the disclosure provides oligomers capable of down-regulating the expression of C9ORF72 by targeting certain complementary nucleobase sequences in the GGGGCC hexanucleotide repeat region of the C9ORF72 gene. In some embodiments, oligomers of the disclosure are capable of down-regulating expression of C9ORF72 RNA transcribed from the sense (plus) strand of the C9ORF72 gene. In other embodiments, oligomers are capable of down-regulating the expression of C9ORF72 RNA transcribed from the antisense (minus) strand of the C9ORF72 gene.

In certain embodiments, the disclosure provides oligomers capable of hybridizing under intracellular conditions to RNA transcribed from the C9ORF72 gene which includes the hexanucleotide repeat region of C9ORF72. In some embodiments, the oligomers target an expanded hexanucleotide repeat region. In some other embodiments, certain oligomers are incapable of targeting C9ORF72 RNA unless it contains two or more hexanucleotide repeats. In some embodiments, the RNA is transcribed from the sense strand of the C9ORF72 gene, and in other embodiments the RNA is transcribed from the antisense strand of the C9ORF72 gene.

Also provided are methods of using the oligomers of the disclosure to treat or prevent diseases associated with expression of C9ORF72 RNA transcripts containing an expansion of the hexanucleotide repeat region. Exemplary diseases that can be treated or prevented include, but are not limited to, frontotemporal dementia (FTD) and amyotrophic lateral sclerosis (ALS). In some embodiments, oligomers of the disclosure down-regulate RNA expression from the C9ORF72 gene, thereby reducing the incidence of intranuclear RNA foci or intracellular aggregates of protein produced from transcripts of the C9ORF72 gene containing hexanucleotide repeats. The oligomers of the disclosure are composed of covalently linked monomers. The term “monomer” includes both nucleosides and deoxynucleosides (collectively, “nucleosides”) that occur naturally in nucleic acids and that contain neither modified sugars nor modified nucleobases, i.e., compounds in which a ribose sugar or deoxyribose sugar is covalently bonded to a naturally-occurring, unmodified nucleobase (sometimes simply called a base) moiety (i.e., the purine and pyrimidine heterocycles adenine, guanine, cytosine, thymine or uracil, abbreviated A, G, C, T, and U, respectively) and “nucleoside analogues,” which are nucleosides that either do occur naturally in nucleic acids or do not occur naturally in nucleic acids, wherein either the sugar moiety is other than a ribose or a deoxyribose sugar (such as bicyclic sugars or 2′ modified sugars, such as 2′ substituted sugars), or the base moiety is modified (e.g., 5-methylcytosine, which can be abbreviated m5C, or C5Me), or both.

An “RNA monomer” is a nucleoside containing a ribose sugar and an unmodified nucleobase.

A “DNA monomer” is a nucleoside containing a deoxyribose sugar and an unmodified nucleobase.

A “Locked Nucleic Acid monomer,” “locked monomer,” or “LNA monomer” is a nucleoside analogue having a bicyclic sugar, as further described herein.

A “modified base”, as used herein, refers to any nucleobase other than adenine, cytosine, guanine, thymidine, or uracil.

As used herein, “5-methylcytosine” refers to a cytosine modified with a methyl group attached to the 5′ position. A 5-methylcytosine is a modified base.

A “2′-O-methoxyethyl group’ (also 2′-MOE and MOE) refers to an O-methoxy-ethyl modification of the 2′ position of a furanosyl ring. A 2′-O-methoxyethyl modified sugar is a modified sugar. Similarly, a “2′-MOE nucleoside” (also 2′-O-methoxyethyl nucleoside), as used herein, refers to a nucleoside comprising a 2′-O-methoxyethyl group.

The terms “corresponding nucleoside analogue” and “corresponding nucleoside” indicate that the base moiety in the nucleoside analogue and the base moiety in the nucleoside are identical. For example, when a “nucleoside” contains a 2-deoxyribose sugar linked to an adenine base moiety, the “corresponding nucleoside analogue” contains, for example, a modified sugar linked to an adenine base moiety.

The terms “oligomer,” “oligomeric compound,” and “oligonucleotide” are used interchangeably in the context of the disclosure, and refer to a molecule formed by covalent linkage of two or more contiguous monomers by, for example, a phosphate group (forming a phosphodiester linkage between adjacent nucleosides) or a phosphorothioate group (forming a phosphorothioate linkage between adjacent nucleosides). In some embodiments, oligomers of the disclosure comprise or consist of 8-50 monomers, such as 10-30 monomers.

In some embodiments, an oligomer comprises nucleosides, or nucleoside analogues, or mixtures thereof as referred to herein. The terms “LNA oligomer” or “LNA oligonucleotide” refer to an oligonucleotide containing one or more LNA monomers.

In various embodiments, oligomers according to the disclosure comprise at least one nucleoside analogue monomer, such as an LNA monomer, or other nucleoside analogue monomer.

Nucleoside analogues optionally included within oligomers may function similarly to corresponding nucleosides, or may have distinct functions. Oligomers in which some or all of the monomers are nucleoside analogues may have improved properties compared to oligomers lacking such analogues making them function better as drugs. Such improved properties may include the ability to cross the blood-brain-barrier, penetrate cell membranes, resistance to extracellular and/or intracellular nucleases and high affinity and specificity for the nucleic acid target. In some embodiments, LNA monomers possess several of the above-mentioned improved properties.

Nucleoside analogues may also be “silent” or “equivalent” in function to the corresponding unmodified nucleoside so that they have no known effect on the way the oligomer functions to inhibit target gene expression and/or its resistance to nucleases. Such “equivalent” nucleoside analogues may nevertheless be useful in that they are easier or less costly to manufacture, be more stable under storage or manufacturing conditions, or serve as an attachment point for a tag or label.

The term “at least one” means integers equal to or larger than 1, such as 1, 2, 3, 4, 5, 6, 7, 8, 9, 10, 11, 12, 13, 14, 15, 16, 17, 18, 19, 20 and so forth. In various embodiments, such as when referring to the nucleic acid targets of the oligomers of the disclosure, the term “at least one” encompasses the terms “at least two” and “at least three” and “at least four,” etc. Similarly, in some embodiments, the term “at least two” comprises the terms “at least three,” “at least four,” etc.

In some embodiments, an oligomer consists of 8-50 contiguously linked monomers, such as 8, 9, 10, 11, 12, 13, 14, 15, 16, 17, 18, 19, 20, 21, 22, 23, 24, 25, 26, 27, 28, 29 or 30 contiguously linked monomers. In other embodiments, an oligomer consists of 10-25 monomers, 10-16 monomers, or 12-16 monomers.

In various embodiments, an oligomer of the disclosure does not comprise RNA monomers (e.g., RNA nucleosides or nucleotides or other RNA monomers). In some embodiments, an oligomer comprises DNA monomers (e.g., DNA nucleosides or nucleotides or other DNA monomers).

When used in reference to the oligomers of the disclosure, the term “region” means a certain number of contiguously linked monomers within the overall oligomer sequence that is less than the overall length of the oligomer, defined as the total number of monomers in the oligomer.

In some embodiments of the disclosure, oligomers are single-stranded, molecules that exclude any substantially self-complementary regions, thereby avoiding or reducing the likelihood that intrastrand base pairing can occur, or that an internal duplex can form. In related embodiments, oligomers are not substantially double-stranded, or are not siRNA.

In some embodiments, the oligomers of the disclosure consist of a contiguously linked stretch of monomers the base sequences of which, and corresponding sequence identification numbers, are disclosed in one or more Tables set forth herein.

### Target Nucleic Acids

The terms “nucleic acid” and “polynucleotide” are used interchangeably herein and are defined as a molecule formed by covalent linkage of two or more monomers as that term is described elsewhere herein. A nucleic acid can be any length, starting with two monomers. The terms nucleic acid and polynucleotide are generic to oligomer, which has structure and length described elsewhere herein. The terms nucleic acid and polynucleotide include, but are not limited to, single-stranded, double-stranded, partially double-stranded, self-complementary and circular nucleic acids and polynucleotides.

As it exists in nature, the human C9ORF72 gene (SEQ ID NO: 187) is located on chromosome 9 at 9p21.2. The reference sequence for the gene has GenBank Accession No. NG_031977.1, which is incorporated by reference. Based on the reference sequence, exon 1 corresponds to bases 5001-5158, exon 2 corresponds to bases 11703-12190, exon 3 corresponds to bases 13277-13336, exon 4 corresponds to bases 16391-16486, exon 5 corresponds to bases 17218-17282, exon 6 corresponds to bases 18568-18640, exon 7 corresponds to bases 20260-20376, exon 8 corresponds to bases 22071-22306, exon 9 corresponds to bases 28160-28217, exon 10 corresponds to bases 30201-30310, and exon 11 corresponds to bases 30445-32322. Exon 1 is non-coding, whereas exons 2-11 have the potential to encode protein depending on the result of alternative mRNA splicing.

To date, three different mRNA transcripts from the C9ORF72 gene have been identified. Transcript variant 1 is predicted to encode a 222 amino acid protein encoded by C9ORF72 exons 2-5 and is called “protein isoform b.” The nucleic acid sequence of transcript variant 1 is set forth as GenBank Accession No. NM_145005.6 (SEQ ID NO:188) and the amino acid sequence of isoform b is set forth as GenBank Accession No. NP_659442.2. Each of these sequences is incorporated by reference herein. In contrast, transcript variants 2 and 3 are predicted to encode the same 481 amino acid protein encoded by C9ORF72 exons 2 through 11 which is called “protein isoform a.” The nucleic acid sequence of transcript variant 2 is set forth as GenBank Accession No. NM_018325.3, the nucleic acid sequence of transcript variant 3 is set forth as GenBank Accession No. NM_001256054.1, and the amino acid sequence of isoform a is set forth as GenBank Accession No. NP_060795.1. Each of these sequences is incorporated by reference herein.

The hexanucleotide (GGGGCC) repeat region, if present, is positioned in the intron located between exons 1a and 1b, usually closer to the first exon. A limited number of repeats of the GGGGCC sequence have been found to be non-pathogenic in humans. As reported in the scientific literature, healthy individuals have an average of two repeats of the GGGGCC sequence, but can possess as many as about 20, whereas expansion to about 30 or more GGGGCC repeats is statistically correlated with developing the neurological diseases ALS or FTD. The typical number of repeats in affected individuals can be many fold higher, however, such as including up to 700 to 1600 hexanucleotide repeats, or more. DeJesus-Hernandez, et al., Neuron 72:245-256 (2011); Renton, et al., Neuron 72:257-268 (2011).

Interestingly, it has been reported that both sense and antisense transcripts from the C9ORF72 gene containing hexanucleotide repeats are elevated in the brains of patients with repeat expansion. M. Kohji, et al., Science 339 (6125):1335-1338 (2013). It was also reported that RNA foci in cells obtained from patients with hexanucleotide repeat expansion contain antisense transcripts. Lagier-Tourenne, PNAS 110(47):E4530-E4539 (2013). Another report found that C9ORF72 antisense transcripts are elevated in the brains of repeat expansion-positive patients and that antisense RNAs accumulate in foci in the brain cells of such patients. Such RNA foci were hypothesized to contribute to the pathogenesis of the ALS and FTD attributed to hexanucleotide repeat expansion.

It was additionally reported that proteins containing dipeptide repeats encoded by the hexanucleotide repeats were translated from sense and antisense transcripts through a mechanism termed repeat-associated non-ATG (RAN) translation. T. Zu, et al., PNAS 110(51):E4968-E4977 (2013). More specifically, proteins containing the dipeptides Pro-Arg, Pro-Ala, and Gly-Pro were translated from the antisense transcripts, whereas proteins containing the dipeptides Gly-Ala, Gly-Arg, and Gly-Pro were translated from the sense transcripts. Because these proteins were observed to collect in cytoplasmic aggregates in affected brain regions, the authors hypothesized the RAN proteins could play a role in pathogenesis causing the ALS or FTD observed in the affected patients.

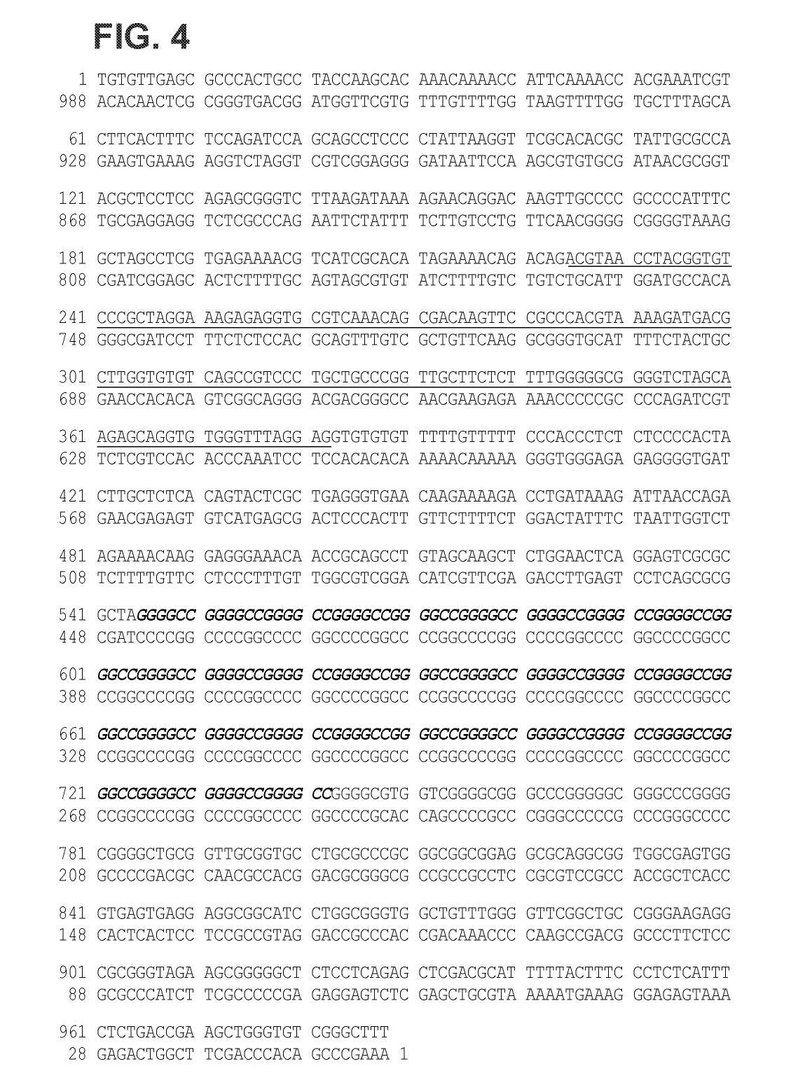

FIG. 4 provides the nucleic acid sequence of the plus strand of an exemplary human C9ORF72 gene containing 33 repeats of the hexanucleotide sequence GGGGCC. The sequence shown is based on that of NCBI reference sequence NG_031977.1, which contains three repeats of the hexanucleotide sequence and one partial repeat (GGGGC). Exon 1 is identified by bold underline font, whereas the hexanucleotide repeat section is identified by bold italic font. The sequence of FIG. 4, however, is merely exemplary and should not be construed as limiting. As discussed further below, other naturally occurring variants or alleles of the C9ORF72 gene exist in human populations, for example, with fewer or a greater number of hexanucleotide repeats. Partial repeats and/or other sequence variants such as single nucleotide polymorphisms (SNPs), or other variants, may also be present adjacent to or outside the hexanucleotide repeat region.

The term “target nucleic acid” refers to the C9ORF72 gene (and naturally occurring variants thereof) as it exists in the genome or as isolated therefrom (such as genomic fragments), including exons and introns (including the hexanucleotide repeat region), genetic control regions (such as promoters and enhancers), splice junctions, and 5′ and 3′ untranslated regions (UTR). Target nucleic acid also includes any RNA transcribed from the plus strand or minus strand of the C9ORF72 gene.

As used herein, “plus strand” of the C9ORF72 gene (and the equivalent terms, “sense,” “coding” or “non-template” strand) refers to the DNA strand of the C9ORF72 gene comprising the same nucleobase sequence (except for thymine in DNA which is replaced by uracil in RNA) as the pre-mRNA which, after splicing, generates mRNA translatable to any of the known C9ORF72 protein isoforms. In contrast, the “minus strand” of the C9ORF72 gene (and the equivalent terms, “antisense,” “non-coding” or “template” strand) refers to the DNA strand of the C9ORF72 gene comprising the nucleobase sequence that is transcribed by cellular RNA polymerase to synthesize the C9ORF72 pre-mRNA described immediately above. The minus strand of the C9ORF72 gene is complementary to the plus strand and vice versa.

RNA transcripts within the term target nucleic acid include C9ORF72 pre-mRNA and mRNA encoding the C9ORF72 protein, including the three known mRNA variants described above, and any new mRNA variants that may yet be discovered. Additionally encompassed by the term target nucleic acid is cDNA prepared from such mRNA.

The present invention contemplates gapmers, uses and compositions thereof, wherein the gapmer is capable of preferentially inhibiting expression of C9ORF72 sense transcript containing an expanded hexanucleotide repeat region when compared with inhibiting expression of a wild-type C9ORF72 sense transcript. As used herein, a “C9ORF72 sense transcript” refers to RNA that is transcribed from the sense strand of the C9ORF72 gene and which also contains the hexanucleotide repeat region found between exons 1a and 1b. As such, the C9ORF72 sense transcript will have areas of substantial sequence identity to the C9ORF72 mRNA sequence, for example, SEQ ID NO:188, but will additionally contain intronic sequence, particularly at least the aforementioned hexanucleotide repeat region.

As used herein, a transcript is said to have an “expanded hexanucleotide repeat region” when it has a pathogenic number of hexanucleotide repeats [for the sense transcript, (GGGGCC)ₙ, and for the antisense transcript, (GGCCCC)ₙ]. Typically, this number is significantly higher than the number of repeats found in the C9ORF72 gene in a normal subject (i.e., a human subject that is not suffering from familiar ALS or FTD), the latter of which generally has 30 or fewer repeats. Expanded hexanucleotide repeat regions of the C9ORF72 gene can be detected using means known in the art. In one example, the number of repeats is determined by sequencing the intronic region between exons 1b and 1b of the C9ORF72 gene. As such, as used herein, an expanded hexanucleotide repeat region is a transcript which has more than 30 hexanucleotide repeats, for example, at least 40 repeats, at least 50 repeats, at least 60 repeats, at least 75 repeats, at least 100 repeats, at least 125, repeats, at least 150 repeats, at least 200 repeats, at least 250 repeats, at least 300 repeats, or more hexanucleotide repeats.

The gapmers of the present invention also are capable of inhibiting expression of a C9ORF72 antisense transcript. As used herein, a “C9ORF72 antisense transcript” refers to RNA that is transcribed from the antisense strand of the C9ORF72 gene and which also contains the hexanucleotide repeat region found between exons 1a and 1b. The C9ORF72 antisense transcript will have areas of substantial sequence complementarity to the C9ORF72 mRNA sequence, for example, SEQ ID NO:188, but additionally contain intron sequences, particularly the hexanucleotide repeat region (GGCCCC)ₙ.

Target nucleic acid also includes RNA transcripts transcribed from the plus (sense) or minus (antisense) strand of the C9ORF72 gene that are not spliced into mRNA encoding C9ORF72 protein. Examples of such RNA transcripts include those present within RNA foci in cells (e.g., brain cells, fibroblasts, white blood cells or other types of cells) from patients with C9ORF72 repeat expansion, as well as RNA transcripts translatable into proteins containing dipeptide repeats encoded by the hexanucleotide repeats, whether translated by repeat-associated non-ATG (RAN) translation or some other mechanism. In some embodiments, proteins containing dipeptide repeats are translated from plus strand RNA transcribed from the antisense strand where the dipeptide repeats are Gly-Ala (e.g., GA or AG), Gly-Pro (e.g., GP or PG) or Gly-Arg (e.g., GR or RG). In other embodiments, proteins containing dipeptide repeats are encoded by minus strand RNA transcribed from the sense strand where the dipeptide repeats are Gly-Pro (e.g., GP or PG), Ala-Pro (e.g., AP or PA) or Pro-Arg (e.g., PR or RP).

Also included within the term “target nucleic acid” are any naturally occurring variants of the plus or minus DNA strands of the C9ORF72 gene and RNA transcribed therefrom, including for example pre-mRNA and mRNA.

The term “naturally occurring variant” refers to any allelic variants of the C9ORF72 gene existing naturally within the human population, and RNA, including pre-mRNA and mRNA, transcribed from such allelic gene variants, and proteins encoded by such allelic gene variants. When referring to the C9ORF72 protein, the term includes naturally occurring forms of the protein (e.g., isoform a or isoform b) and proteins having undergone co-translational or post-translational processing, such as signal peptide cleavage, proteolytic cleavage, or glycosylation. Naturally occurring variants of the C9ORF72 protein also includes proteins comprising repeating dipeptide sequences resulting from repeat associated non-ATG (RAN) translation from RNA transcribed from the C9ORF72 gene, as described more fully in Mori, et al., Science 339:1335-1338 (2013), and Ash, et al., Neuron 77, 639-646 (2013).

Of particular relevance here are naturally occurring variants include alleles of the C9ORF72 gene containing a pathogenic number of GGGGCC hexanucleotide repeats. Naturally occurring variants also includes RNA transcribed from the antisense strand comprising such hexanucleotide repeats (including pre-mRNA and mRNA), as well as RNA transcribed from the sense strand comprising the reverse complement of the hexanucleotide repeats (i.e., GGCCCC).

In some embodiments the nucleic acid sequence of the expanded region is provided by the formula (GGGGCC)n, or (GGCCCC)n wherein GGGGCC or GGCCCC is a single hexanucleotide repeat unit, and wherein “n” is a number that varies from 0 to 5000 or more. In certain embodiments, n can be 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000, 2000, 3000, 4000, 5000, or more, including any integer between or among the previously specified values.

The nucleobase sequence of a hexanucleotide repeat unit depends on which strand is being read. In the plus strand, the repeated hexanucleotide sequence is 5′-GGGGCC-3′ and the sequence would be the same in any RNA transcribed from the complementary minus strand. Conversely, in the minus strand, the repeated hexanucleotide sequence is 5′-GGCCCC-3′ (i.e., the reverse complement) and the sequence would be the same in any RNA transcribed from the complementary plus strand. While the start site of the RNA transcribed from the “minus” or antisense strand of C9ORF72 has not been identified, it is known to contain the hexanucleotide repeat [albeit in the complementary sequence (i.e., GGCCCC)], as both sense and antisense transcripts containing the intron 1 sequence (where the GGGGCC repeat is located) were found to be strongly increased in C9ORF72 patients (Mori et al., ibid), and nuclear RNA foci containing GGCCCC-containing repeats have been identified in fibroblasts from C9ORF72 patients (Lagier-Tourenne et al., ibid)

According to some embodiments, when viewed from the perspective of the plus strand, the nucleic acid sequence of the repeat region (in DNA or RNA) is provided by the formula (GGGGCC)ₙ, wherein GGGGCC is a single hexanucleotide repeat unit and wherein “n” is an integer that varies from 0 to 5000 or more. In other embodiments, when viewed from the perspective of the minus strand, the nucleic acid sequence of the expanded region is provided by the formula (GGCCCC)ₙ, wherein GGCCCC is a single hexanucleotide repeat unit, and wherein “n” is an integer that varies from 0 to 5000 or more.

In certain embodiments, “n” in the formulae above can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000, 2000, 3000, 4000, 5000, or more, including any integer between or among the previously specified values.

In some embodiments, the hexanucleotide repeat region can be flanked on either the 5′ or 3′ end, or both the 5′ and 3′ ends, by a single partial repeat. If on the plus strand, a partial repeat positioned at the 5′ end of the repeat region can have the sequence GGGCC, GGCC, GCC, CC, and C, and a partial repeat positioned at the 3′ end of the repeat region can have the sequence GGGGC, GGGG, GGG, GG, and G. From the perspective of the minus strand, a partial repeat positioned at the 5′ end of the repeat region can have the sequence, GCCCC, CCCC, CCC, CC, or C, and a partial repeat positioned at the 3′ end of the repeat region can have the sequence GGCCC, GGCC, GGC, GG, or G.

In certain embodiments, oligomers of the disclosure can hybridize with a nucleobase sequence present in the plus strand of the C9ORF72 gene or corresponding RNA transcript. In other embodiments, oligomers of the disclosure can hybridize with a nucleobase sequence present in the minus strand of the C9ORF72 gene or corresponding RNA transcript. And in yet other embodiments, oligomers of the disclosure can hybridize with a nucleobase sequence present in both the plus strand and minus strand of the C9ORF72 gene or corresponding RNA transcript. In the latter embodiments, the sequences in the plus and minus strands targeted by the oligomers need not be identical.

Oligomers of the disclosure hybridize to a region in the target nucleic acid (the “target region”) having a sequence of nucleobases complementary or partially complementary to those in the oligomer. Accordingly, the sequence of nucleobases in the oligomer is identical or substantially identical to the reverse complement sequence of the region to which the oligomer hybridizes. If the sequence of the oligomer and the reverse complement sequence of the target region are identical, then the oligomer is “complementary” to the target region. If, however, the sequence of the oligomer and the reverse complement sequence of the target region are similar but not identical, then the oligomer is “partially complementary” to the target region.

Without wishing to be bound by any particular theory of operation, hybridization typically occurs by Watson-Crick base pairing, although Hoogsteen hydrogen bonding and reversed Hoogsteen hydrogen bonding may also be possible. The mechanism by which hybridization may occur in any particular case, however, is not intended to be limiting in any way.

Usually, an oligomer's target region is the nucleobase sequence in a target nucleic acid having the highest percentage complementarity to the oligomer. The percentage complementarity between the oligomer and its target region can be calculated by counting the number of identical bases between the oligomer sequence and the reverse complement sequence of the target region, dividing by the total length of the oligomer, and then multiplying the quotient by 100. In some embodiments, however, an oligomer may be able to hybridize to a target region even when the percentage complementarity is less than 100% by virtue of the existence of one or more mismatches between the complementary sequences of the oligomer and target region.

As used herein, “mismatch” refers to nonidentity of a nucleobase between two sequences under comparison, for example, as between the nucleobase sequence of an oligomer and the reverse complement of the target region to which it hybridizes. Mismatches can consist of a single nucleobase difference or a plurality of nucleobase differences. Typically, mismatches are considered in the context of larger sequences that are otherwise substantially similar.

Identifying an oligomer's target region or regions in a target nucleic acid can efficiently be carried out using computer bioinformatics software implementing a sequence alignment algorithm. A variety of such programs and algorithms are known in the art, which can also calculate percentage complementarity (also called percentage identity or percentage homology). A non-limiting example is software implementing the Smith-Waterman algorithm available from the European Bioinformatics Institute (http://www.ebi.ac.uk/Tools/emboss/) or from the BLAST software suite available from National Institute of Medicine (http://blast.ncbi.nlm.nih.gov/Blast.cgi).

In various embodiments, oligomers of the disclosure are capable of reducing the amount of RNA that is transcribed from the C9ORF72 gene, or otherwise inhibiting the expression of such RNA. In some embodiments, the RNA is a C9ORF72 sense transcript. In other embodiments, the RNA is a C9ORF72 antisense transcript. In particular embodiments, the oligomers of the present invention are capable of inhibiting both a C9ORF72 sense transcript and C9ORF72 antisense transcript. As a consequence of reducing the amount of RNA transcribed from the C9ORF72 gene, oligomers of the disclosure can reduce the average size and/or number of RNA foci containing such transcripts in the cells of patients with ALS, FTD, or other disease or disorder attributed to hexanucleotide repeat expansion in the C9ORF72 gene. Oligomers of the disclosure can also reduce the amount of pre-mRNA and mRNA encoding C9ORF72 protein in its various isoforms.

In certain embodiments, the gene and RNA contain hexanucleotide repeats as defined herein. In particular embodiments, the gapmers of the present invention are capable of preferentially inhibiting expression of a C9ORF72 sense transcript containing an expanded hexanucleotide repeat when compared with inhibiting expression of a wild-type C9ORF72 sense transcript. For example, in one embodiment, the oligomers of the present invention with a half-maximal inhibitory concentration (IC₅₀) for a C9ORF72 sense transcript containing an expanded hexanucleotide repeat that is at least 50% less than the IC50 for a wild-type C9ORF72 sense transcript (i.e., a transcript containing, for example 20 or fewer hexanucleotide repeats, for example, 15 or fewer, 10 or fewer, 8 or fewer, or less, hexanucleotide repeats). As such, in certain embodiments, the gapmer of the present invention can have an IC₅₀ for the C9ORF72 sense transcript containing an expanded hexanucleotide repeat that is 25% or less, 20% or less, 15% or less, 10% or less, 5% or less, 3% or less, 2% or less, 1% or less than the IC50 for a wild-type C9ORF72 sense transcript (i.e., not containing an expanded hexanucleotide repeat). Methods for determining the IC50 of an oligomer for inhibition of expression of a C9ORF transcript are described hereinbelow in the Examples section. Typically, the IC₅₀ is determined in vitro using cells, for example, fibroblasts or kidney cells, that contain either a wild-type human C9ORF72 or one containing expanded hexanucleotide repeats (for example, fibroblasts such as ND40063 cells, which were isolated from patients with familial FTD/ALS and confirmed to have expanded hexanucleotide repeats). Oligomers are provided to the cells at defined concentrations and delivered into the cells using any number of methods known in the art. For example, oligomers can be introduced by gymnotic delivery (see Example 1; see also Soifer, H. S., et. al., (2012) Methods Mol Biol.; 815:333-46, which is incorporated herein by reference in its entirety), transfection (Graff J R, et al., (2007) J Clin Invest 117:2638-2648, incorporated herein by reference in its entirety), or electroporation (Carroll, J. B. et al., (2011) Mol. Ther. 19(12): 2178-2185, incorporated herein by reference in its entirety). A certain time after the oligomer is delivered into the cells (for example, 72 hrs post-transfection), RNA is extracted and the relative amounts of the C9ORF72 sense transcript and/or C9ORF72 antisense transcript are measured and compared to an internal control using methods known in the art (e.g., RT-PCR, RNA-SEQ. etc.) using primers designed to detect the respective transcripts (SEQ ID Nos: 179-184; see Example 1). The IC50 is determined by plotting the relative abundance of the transcript against the concentration, and establishing the concentration at which 50% of maximal inhibition occurs. In one embodiment, the ability of an oligomer, including the gapmers of the present invention to preferentially inhibit expression of a C9ORF72 sense transcript with an expanded hexanucleotide repeat region when compared with a wild-type C9ORF72 sense transcript is measured using ND40063 and wild-type human fibroblast cells, by gymnotic delivery of oligomers, and detecting transcript levels by RT-PCR 72 hrs after delivery, using the primers and conditions described in the Examples section. Methods described above can also be used to determine the ability of an oligomer, including the gapmers of the present invention, to inhibit expression of a C9ORF72 antisense transcript.

As a consequence of reducing RNA accumulation, oligomers of the disclosure can indirectly reduce the amount of protein, if any, encoded by the transcribed RNA. In some embodiments, the protein contains dipeptide repeats encoded by the hexanucleotide repeat region in the C9ORF72 gene, whether from the plus or minus strand, or from both strands. In other embodiments, the protein is C9ORF72 protein in its various isoforms. By reducing the amount of protein translated from RNA transcribed from the C9ORF72 gene, oligomers of the disclosure can reduce the average size and/or number of intracellular aggregates comprising such proteins accumulating in the cells of patients with ALS, FTD, or other disease or disorder attributed to hexanucleotide repeat expansion in the C9ORF72 gene.

In some embodiments, oligomers of the disclosure function to reduce the amount of RNA that is transcribed from the C9ORF72 gene by hybridizing with a target region in the C9ORF72 gene (in the plus and/or minus strand) and inhibiting transcription of the strand containing the target region into RNA. In some other embodiments, oligomers of the disclosure function to reduce the amount of RNA that is transcribed from the C9ORF72 gene by hybridizing with a target region in RNA that has already been transcribed from the plus and/or minus strand of the C9ORF72 gene. Examples of such RNA include unspliced RNA, pre-mRNA or mRNA. In certain embodiments, oligomers of the disclosure promote the destruction of transcribed RNA by endogenous nucleases, and/or inhibit or prevent translation of the RNA into protein. Other mechanisms of reducing the amount of RNA that is transcribed from the C9ORF72 gene by the oligomers of the disclosure are possible and the mechanism of action is not intended to limit the scope of the invention in any respect.

In various embodiments, oligomers of the disclosure reduce the amount RNA that has already been transcribed from the minus strand of the C9ORF72 gene (e.g., unspliced RNA, pre-mRNA or mRNA) in a cell at least 10%, 20%, 30%, 40%, 50%, 60%, 70%, 80%, 90% or 95% or more as compared to the amount of C9ORF72 RNA that would be present in the absence of the oligomers. In other embodiments, oligomers of the disclosure reduce the amount of RNA transcription from the minus strand of the C9ORF72 gene in a cell at least 10%, 20%, 30%, 40%, 50%, 60%, 70%, 80%, 90% or 95% or more as compared to the amount of transcription in the absence of the oligomers. In other embodiments, oligomers of the disclosure reduce the amount C9ORF72 protein in a cell at least 10%, 20%, 30%, 40%, 50%, 60%, 70%, 80%, 90% or 95% or more as compared to the amount of C9ORF72 protein that would be present in the absence of the oligomers.

In various embodiments, oligomers of the disclosure reduce the amount RNA that has already been transcribed from the plus strand of the C9ORF72 gene in a cell (e.g., unspliced RNA, pre-mRNA or mRNA) by at least 10%, 20%, 30%, 40%, 50%, 60%, 70%, 80%, 90% or 95% or more as compared to the amount of C9ORF72 RNA that would otherwise be present in the cell the absence of the oligomers. In other embodiments, oligomers of the disclosure reduce the amount of RNA transcription from the plus strand of the C9ORF72 gene in a cell at least 10%, 20%, 30%, 40%, 50%, 60%, 70%, 80%, 90% or 95% or more as compared to the amount of transcription in the absence of the oligomers.

Techniques for measuring the amount of C9ORF72 RNA in cells, or the amount of C9ORF72 RNA transcribed or translated into C9ORF72 protein, are familiar to those of ordinary skill in the art. For example, the amount of C9ORF72 RNA can be detected using Northern blots, or more sensitive techniques such as quantitative RT-PCR and related techniques. And, for example, the amount of C9ORF72 protein can be measured using Western blots, ELISA or RIA using an antibody that specifically binds C9ORF72 protein. Other techniques that can be used are also well-known in the art.

The efficiency with which a particular oligomer is effective to reduce or inhibit the amount of C9ORF72 RNA in a cell depends on a variety of factors, such as its length, melting temperature and ability to recruit RNase H, and possibly other factors. Irrespective of the underlying mechanism(s), the inhibitory efficiency of an oligomer may be determined empirically and expressed as the 50% inhibitory concentration, abbreviated IC₅₀.

According to one embodiment, the IC₅₀ value of an oligomer can be determined by treating test cells expressing the C9ORF72 gene, for example certain human cells in vitro, with a range of concentrations of the oligomer and then determine what concentration would reduce the amount of C9ORF72 RNA, transcription and/or translation by 50% compared to untreated control cells. Methods for performing such experiments are known in the art. In some embodiments, oligomers can be introduced into test cells using a transfection reagent such as lipofectamine. Alternatively, no transfection reagent is used, but rather the oligomers are simply added to the cells in a physiologically compatible fluid, such as PBS or growth medium. An example of this approach is called gymnosis. See, e.g., Stein, C. A., et al., Nuc. Acids Res., 38(1):e3, pp. 1-8 (2010). In some embodiments, the test cells are fibroblasts or other cells isolated from human patients diagnosed with familial ALS or familial FTD caused by hexanucleotide repeat expansion.

In some embodiments, oligomers of the disclosure have an IC₅₀ value in the micromolar range, e.g., 1-10 uM, 5-20 uM, 10-20 uM, 15-25 uM, 20-30 uM, 25-35 uM, 30-40 uM, 35-45 uM, 40-50 uM, 45-55 uM, 50-60 uM, 55-65 uM, 60-70 uM, 65-75 uM, 70-80 uM, 75-85 uM, 80-90 uM, 85-95 uM, 90-100 uM, 100-150 uM, 150-200 uM, 200-300 uM, 300-400 uM, 400-500 uM or more. In some other embodiments, oligomers of the disclosure have an IC₅₀ value in the nanomolar range, e.g., 1-10 nM, 5-20 nM, 10-20 nM, 15-25 nM, 20-30 nM, 25-35 nM, 30-40 nM, 35-45 nM, 40-50 nM, 45-55 nM, 50-60 nM, 55-65 nM, 60-70 nM, 65-75 nM, 70-80 nM, 75-85 nM, 80-90 nM, 85-95 nM, 90-100 nM, 100-150 nM, 150-200 nM, 200-300 nM, 300-400 nM, 400-500 nM or more. And, in yet other embodiments, oligomers of the disclosure have an IC₅₀ value in the picomolar range, e.g., 30-40 pM, 35-45 pM, 40-50 pM, 45-55 pM, 50-60 pM, 55-65 pM, 60-70 pM, 65-75 pM, 70-80 pM, 75-85 pM, 80-90 pM, 85-95 pM, 90-100 pM, 100-150 pM, 150-200 pM, 200-300 pM, 300-400 pM, 400-500 pM, 500-600 pM, 600-700 pM, 700-800 pM, 800-900 pM, or 900-1000 pM. It is noted that IC50 values can vary depending on the ability of the oligomers to enter the cell. Thus, in one embodiment, IC50 values are determined in vitro in cells after oligomer delivery using a method selected from gymnotic delivery, using transfection reagents containing cationic lipid or lipid derivative, or electroporation. In one embodiment, IC50 values can be determined after gymnotic delivery of the oligomers.

In some embodiments oligomers of the disclosure have an IC₅₀ value in the submicromolar range, i.e., less than 1 uM, less than 500 nM, less than 250 nM, less than 100 nM, less than 50 nM, less than 10 nM, less than 5 nM or less than 2 nM. In some other embodiments oligomers of the disclosure have an IC₅₀ value in the subnanomolar range, i.e., less than 1 nM, less than 500 pM, less than 250 pM, less than 100 pM, less than 50 pM, or less than 10 pM.

The disclosure additionally provides methods of inhibiting expression from the C9ORF72 gene. According to an embodiment, the disclosure provides a method for inhibiting C9ORF72 expression by contacting a cell expressing the C9ORF72 gene with an amount of an oligomer of the disclosure effective to inhibit expression of the C9ORF72 gene. In some embodiments, the oligomer hybridizes to a target region on the plus and/or minus strand of the C9ORF72 gene, or RNA produced from the plus and/or minus strand.

Exemplary embodiments of oligomers of the disclosure capable of hybridizing to target regions in the human C9ORF72 gene (plus and/or minus strand), or RNA produced therefrom, are disclosed in TABLE 4 in the Examples section.

In related embodiments, the disclosure provides oligomers having a nucleobase sequence that differs by 1 or more, 2 or more, 3 or more, or 4 or more bases (i.e., mismatches relative to complementary target regions) from those oligomers listed in TABLE 4, yet are still capable of hybridizing to the C9ORF72 gene or RNA produced therefrom and thereby inhibit production or stability of C9ORF72 RNA. In some embodiments, the destabilizing effect of mismatches on duplex stability may, for example, be compensated for by increased oligomer length and/or increased number of nucleoside analogues, such as LNA monomers, present within the oligomer. In other embodiments, the number of mismatches is 4 or less, 3 or less, 2 or less, or only 1. In yet other embodiments, the disclosure includes oligomers at least 80%, at least 85%, at least 90%, at least 91%, at least 92%, at least 93%, at least 94%, at least 95%, at least 96%, at least 97%, at least 98%, or at least 99% identical to the oligomers specifically exemplified by in Table 4 having SEQ ID NO:1 to SEQ ID NO:176, wherein said similar oligomers are still capable of hybridizing to the C9ORF72 gene or RNA produced therefrom and thereby inhibit production or stability of C9ORF72 RNA.

In some embodiments, the oligomer comprises additional monomers at the 5′ end only, the 3′ end only or at both 5′ and 3′ ends that are non-complementary to the sequence of the target region. In some embodiments, as many as 5 additional monomers, e.g., 1, 2, 3, 4 or 5 additional monomers, can be placed at either the 5′ end, the 3′ end, or both 5′ and 3′ ends. In this way, an oligomer can comprise a region that is complementary to a target region in the C9ORF72 gene, as well as non-complementary flanking region at the 5′ and/or 3′ end of the oligomer. In certain embodiments, the flanking non-complementary monomers are DNA or RNA monomers, for example, nucleotides or nucleosides.

Oligomers of the disclosure may be synthesized using any technique familiar to those of ordinary skill, for example, the technique of solid phase synthesis.

### Oligomer Length

In some non-limiting embodiments, the oligomer comprises or consists of 10-50, 11-48, 12-46, 13-44, 14-42, 15-40, 16-38, 17-36, 18-34, 19-32, 20-30, 21-28, 22-26 contiguous monomers. In other embodiments, oligomers comprise or consist of 10-16, 10-18, 10-22, 10-24, 10-25, 12-14, 12-16, 12-18, 12-24, 12-25, 13-17, 14-16, or 14-18 contiguous monomers. In other embodiments, the oligomer comprises or consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous monomers. In some embodiments, the oligomer consists of no more than 22, 21, 20, 19, 18, 17, 16, 15 or fewer contiguous monomers. In particular embodiments, the oligomers of the present invention, including gapmers described herein, consist of between 12 and 20 monomers.

### Nucleosides and Nucleoside Analogues

In various embodiments, at least one of the monomers of the oligomer is a modified nucleoside, also called a nucleoside analogue. In some embodiments, the modified nucleoside analogue contains a modified base, such as a base selected from the group consisting of 5-methylcytosine (which can be abbreviated m5C, or C5Me), isocytosine, pseudoisocytosine, 5-bromouracil, 5-propynyluracil, 6-aminopurine, 2-aminopurine, inosine, diaminopurine, and 2-chloro-6-aminopurine, xanthine, hypoxanthine, isocytosine, pseudoisocytosine, 5-bromouracil, 5-propynyluracil, 6-aminopurine, 2-aminopurine, inosine, diaminopurine, and 2-chloro-6-aminopurine.

In various embodiments, at least one of the monomers of the oligomer is a nucleoside analogue that contains a modified sugar.

In some embodiments, the linkage between at least two contiguous monomers of the oligomer is other than a phosphodiester bond.

In certain embodiments, oligomers include at least one monomer that has a modified base, at least one monomer having a modified sugar (which may be the same monomer having the modified base), and at least one inter-monomer linkage that is other than a phosphodiester bond. Specific examples of nucleoside analogues useful in the oligomers described herein are described by e.g. Freier & Altmann; Nucl. Acid Res., 1997, 25, 4429-4443 and Uhlmann; Curr. Opinion in Drug Development; 2000, 3(2), 293-213, and in Scheme 1 (in which some nucleoside analogues are shown in the form of nucleotides):

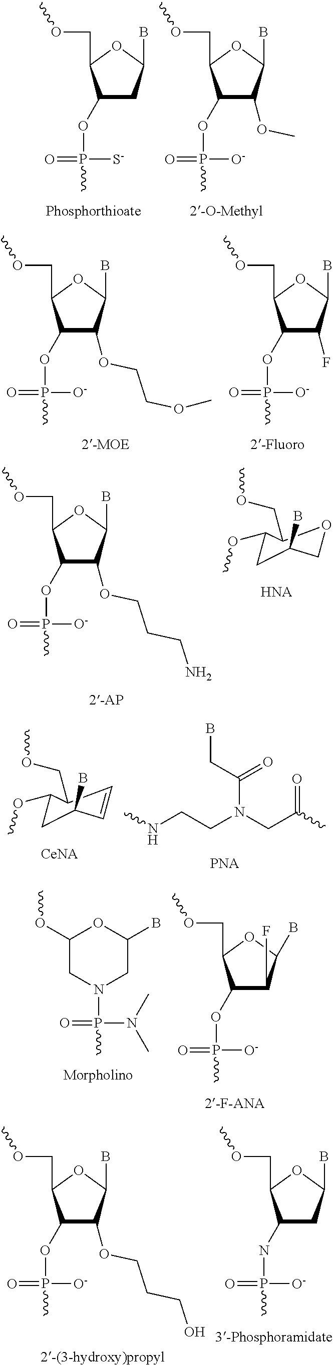

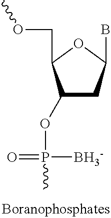

The oligomer may thus comprise or consist of a sequence of linked nucleosides, including for example DNA or RNA, or a combination of nucleosides and one or more nucleoside analogues. In some embodiments, such nucleoside analogues suitably enhance the affinity of the oligomer for the target region of the target nucleic acid.

Examples of suitable and preferred nucleoside analogues are described in WO 2007/031091, or are referenced therein.

In some embodiments, the nucleoside analogue comprises a sugar moiety modified to provide a 2′-substituent group, such as 2′-O-alkyl-ribose sugars, 2′-amino-deoxyribose sugars, and 2′-fluoro-deoxyribose sugars.

In some embodiments, the nucleoside analogue comprises a sugar in which a bridged structure, creating a bicyclic sugar (LNA), is present, which can enhance binding affinity and may also provide some increased nuclease resistance. In various embodiments, the LNA monomer is selected from oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). In certain embodiments, the LNA monomers are beta-D-oxy-LNA. LNA monomers are further described, below.

Incorporation of affinity-enhancing nucleoside analogues in the oligomer, such as LNA monomers or monomers containing 2′-substituted sugars, can allow the size of the oligomer to be reduced, and may also reduce the upper limit to the size of the oligomer before non-specific or aberrant binding takes place.

In certain embodiments, the oligomer comprises at least 2 nucleoside analogues. In some embodiments, the oligomer comprises from 3-8 nucleoside analogues, e.g. 6 or 7 nucleoside analogues. In preferred embodiments, at least one of the nucleoside analogues is a locked nucleic acid (LNA) monomer; for example at least 3 or at least 4, or at least 5, or at least 6, or at least 7, or 8, of the nucleoside analogues are LNA monomers. In some embodiments all the nucleoside analogues are LNA monomers.

It will be recognized that when referring to a preferred oligomer base sequence, in certain embodiments the oligomers comprise a corresponding nucleoside analogue, such as a corresponding LNA monomer or other corresponding nucleoside analogue, which raise the duplex stability, e.g., melting temperature (Tm), of the oligomer/target region duplex (i.e., affinity enhancing nucleoside analogues).

In various preferred embodiments, any mismatches (that is, noncomplementarities) between the base sequence of the oligomer and the base sequence of the target region, if present, are located other than in the regions of the oligomer that contain affinity-enhancing nucleoside analogues (e.g., regions A or C), such as within region B as referred to herein below, and/or within region D as referred to herein below, and/or in regions of the oligomer containing only nucleosides, and/or in regions which are 5′ or 3′ to the region of the oligomer that is complementary to the target region.

In some embodiments the nucleoside analogues present within the oligomer of the disclosure (such as in regions A and C mentioned herein) are independently selected from, for example: monomers containing 2′-O-alkyl-ribose sugars, monomers containing 2′-amino-deoxyribose sugars, monomers containing 2′-fluoro-deoxyribose sugars, LNA monomers, monomers containing arabinose sugars (“ANA monomers”), monomers containing 2′-fluoro-ANA sugars, monomers containing d-arabino-hexitol sugars (“HNA monomers”), intercalating monomers as defined in Christensen, Nucl. Acids. Res. 30:4918-4925 (2002), and monomers containing 2′-O-methyl (2′-OMe) or 2′-O-methoxyethyl (2′-MOE) ribose sugars. In certain embodiments, there is only one of the above types of nucleoside analogues present in the oligomer of the disclosure, or region thereof.

In certain embodiments, the nucleoside analogues contain 2′-O-methoxyethyl-ribose sugars (2′-MOE), or 2′-fluoro-deoxyribose sugars or LNA sugars, and as such the oligonucleotide of the disclosure may comprise nucleoside analogues which are independently selected from these three types of analogue, or may comprise only one type of analogue selected from the three types. In certain oligomer embodiments containing nucleoside analogues, at least one of the nucleoside analogues contains a 2′-MOE-ribose sugar, such as 2, 3, 4, 5, 6, 7, 8, 9 or 10 nucleoside analogues containing 2′-MOE-ribose sugars. In certain embodiments, at least one of the nucleoside analogues contains a 2′-fluoro deoxyribose sugar, such as 2, 3, 4, 5, 6, 7, 8, 9 or 10 nucleoside analogues containing 2′-fluoro-deoxyribose sugars.

In various embodiments, the oligomer according to the disclosure comprises at least one Locked Nucleic Acid (LNA) monomer, such as 1, 2, 3, 4, 5, 6, 7, or 8 LNA monomers, such as 3-7 or 4-8 LNA monomers, or 3, 4, 5, 6 or 7 LNA monomers. In various embodiments, all of the nucleoside analogues are LNA monomers. In some embodiments, the oligomer comprises both beta-D-oxy-LNA monomers, and one or more of the following LNA units: thio-LNA monomers, amino-LNA monomers, oxy-LNA monomers, and/or ENA monomers in either the beta-D or alpha-L configuration or combinations thereof. In certain embodiments, the cytosine base moieties of all LNA monomers in the oligomer are 5-methylcytosines. In certain embodiments of the disclosure the oligomer comprises both LNA and DNA monomers. Typically, the combined total of LNA and DNA monomers is 10-25, preferably 10-20, even more preferably 12-16. In certain embodiments of the disclosure the oligomer, or a region thereof, consists of at least one LNA monomer, and the remaining monomers are DNA monomers. In certain embodiments, the oligomer comprises only LNA monomers and nucleosides (such as RNA or DNA monomers, most preferably DNA monomers), optionally linked with modified linkage groups such as phosphorothioate.

In various embodiments, nucleosides or nucleoside analogues of the oligomer have one of the naturally occurring bases found in DNA or RNA, that is, adenine, guanine, cytosine, thymidine, or uracil. In other embodiments, nucleoside analogues of the oligomer have a base that does not naturally occur in DNA or RNA, or that is a modified base, non-limiting examples of which include xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA Monomers

The term “LNA monomer” refers to a nucleoside analogue containing a bicyclic sugar (an “LNA sugar”). The terms “LNA oligomer” and “LNA oligonucleotide” refer to an oligomer containing one or more LNA monomers.

According to some embodiments, the LNA monomer used in the oligomers of the disclosure has the following structure (formula I):

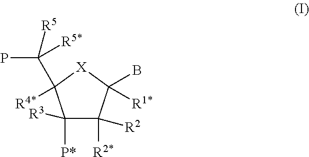

wherein X is selected from —O—, —S—, —N(R^{N*})—, —C(R⁶R^{6*})—;
B is selected from hydrogen, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;
P designates the radical position for an internucleoside linkage to a succeeding monomer, or a 5′-terminal group, such internucleoside linkage or 5′-terminal group optionally including the substituent R⁵ or equally applicable the substituent R^{5*};
P* designates an internucleoside linkage to a preceding monomer, or a 3′-terminal group;
R^{4*} and R^{2*} together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryl-oxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents Ra and R^{b} together may designate optionally substituted methylene (═CH₂), and each of the substituents R^{1*}, R², R³, R⁵, R^{5*}, R⁶ and R^{6*}, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof. For all chiral centers, asymmetric groups may be found in either R or S orientation

In certain embodiments, R^{5*} is selected from H, —CH₃, —CH₂—CH₃, —CH₂—O—CH₃, and —CH═CH₂.

In certain embodiments, R^{4*} and R² together designate a biradical selected from —C(R^{a}R^{b})—O, —C(R^{a}R^{b})—C(R^{c}R^{d})—O, —C(R^{a}R^{b})—C(R^{c}R^{d})—C(R^{e}R^{f})—O, —C(R^{a}R^{b})—O—C(R^{c}R^{d})—, —C(R^{a}R^{b})—O—C(R^{c}R^{d})—O, —C(R^{a}R^{b})—C(R^{c}R^{d})—, —C(R^{a}R^{b})—C(R^{c}R^{d})—C(R^{e}R^{f})—, —C(R^{a})═C(R^{b}) C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryl-oxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate a substituted methylene (═CH₂). For all chiral centers, asymmetric groups may be found in either R or S orientation.

In certain embodiments, R^{4*} and R^{2*} together designate a biradical selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, CH(CH₂—O—CH₃)—O—, —O—CH(CH₂OCH₃)—, —O—CH(CH₂CH₃)—, —O—CH(CH₃)—, and —O—CH₂—O—CH₂—.

For all chiral centers, asymmetric groups may be found in either R or S orientation.

In some other embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1*}, R², R³, R⁵, R^{5*} are hydrogen. For all chiral centers, asymmetric groups may be found in either R or S orientation.

In certain embodiments, R^{1*}, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1*}, R², R³ are hydrogen. For all chiral centers, asymmetric groups may be found in either R or S orientation.

In certain embodiments, R⁵ and R^{5*} are each independently selected from the group consisting of H, —CH₃, —CH₂—CH₃, —CH₂—O—CH₃, and —CH═CH₂. In some embodiments, either R⁵ or R^{5*} are hydrogen, whereas the other group (R⁵ or R^{5*} respectively) is selected from the group consisting of C₁₋₅ alkyl, C₂₋₆ alkenyl, C₂₋₆ alkynyl, substituted C₁₋₆ alkyl, substituted C₂₋₆ alkenyl, substituted C₂₋₆ alkynyl or substituted acyl (—C(═O)—), wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, OJ₁, SJ₁, NJ₁J₂, N₃, COOJ₁, CN, O—C(═O)NJ₁J₂, N(H)C(═NH)NJ,J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, C₁₋₆ aminoalkyl, substituted C₁₋₆ aminoalkyl or a protecting group. In some embodiments either R⁵ or R^{5*} is a substituted C₁₋₆ alkyl. In some embodiments either R⁵ or R^{5*} is a substituted methylene wherein exemplary substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R^{5*} is methyl, ethyl or methoxymethyl. In some embodiments either R⁵ or R^{5*} is methyl. In a further embodiment either R⁵ or R^{5*} is ethylenyl. In some embodiments either R⁵ or R^{5*} is substituted acyl. In some embodiments either R⁵ or R^{5*} is C(═O)NJ₁J₂. For all chiral centers, asymmetric groups may be found in either R or S orientation. Exemplary 5′ modified bicyclic nucleotides are also disclosed in WO 2007/134181.

In certain embodiments B is a nucleobase, including nucleobase analogues and naturally occurring nucleobases, such as a purine or pyrimidine, or a substituted purine or substituted pyrimidine. In certain non-limiting embodiments B can be a nucleobase selected from the group consisting of adenine, cytosine, thymine, adenine, uracil, or a modified or substituted nucleobase, such as 5-thiazolo-uracil, 2-thio-uracil, 5-propynyl-uracil, 2-thio-thymine, 5-methylcytosine, 5-thiozolo-cytosine, 5-propynyl-cytosine, and 2,6-diaminopurine.

In some other embodiments, R^{4*} and R^{2*} together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, substituted C₁₋₁₂-alkyl, substituted C₂₋₁₂-alkenyl, substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In certain embodiments R^{4*} and R^{2*} together designate a biradical selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, —CH(CH₂—O—CH₃)—O—, —CH₂—CH₂—, and —CH═CH—. For all chiral centers, asymmetric groups may be found in either R or S orientation.

In some other embodiments, R^{4*} and R^{2*} together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆alkoxyl, substituted C₁₋₆alkoxyl, acyl, substituted acyl, C₁₋₆ aminoalkyl or substituted C₁₋₆ aminoalkyl, such as hydrogen, and; wherein R^{c} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen. For all chiral centers, asymmetric groups may be found in either R or S orientation.

In some other embodiments, R^{4*} and R^{2*} together designate the biradical C(R^{a}R^{b})—O—C(R^{c}R^{d})—O—, wherein R^{a}, R^{b}, R^{c}, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen. For all chiral centers, asymmetric groups may be found in either R or S orientation.

In some other embodiments, R^{4*} and R^{2*} form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments said substituent group is C₁₋₆ alkoxy. In some embodiments Z is CH₃OCH₂—. For all chiral centers, asymmetric groups may be found in either R or S orientation. Exemplary bicyclic nucleotides are disclosed in U.S. Pat. No. 7,399,845. In some embodiments, R^{1*}, R², R³, R⁵, R^{5*} are hydrogen. In some embodiments, R^{1*}, R², R^{3*} are hydrogen, and one or both of R⁵, R^{5*} may be other than hydrogen as referred to above and as disclosed in WO 2007/134181. For all chiral centers, asymmetric groups may be found in either R or S orientation.

In some other embodiments, R^{4*} and R^{2*} together designate a biradical which comprise a substituted amino group in the bridge such as consist or comprise of the biradical —CH₂—N(R^{c})—, wherein R^{c} is C₁₋₁₂ alkyloxy. In some embodiments R^{4*} and R^{2*} together designate a biradical —Cq₃q₄-NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)N J₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Exemplary bicyclic nucleotides are disclosed in WO2008/150729.

In some other embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1*}, R², R³, R⁵, R^{5*} are hydrogen. In some embodiments, R^{1*}, R², R³ are hydrogen and one or both of R⁵, R^{5*} may be other than hydrogen as referred to above and in WO 2007/134181. In some embodiments R^{4*} and R^{2*} together designate a biradical (bivalent group) C(R^{a}R^{b})—O—, wherein R^{a} and R^{b} are each independently halogen, C₁-C₆ alkyl, substituted C₁-C₆ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁SJ₁, SOJ₁, SO₂J₁, NJ₁J₂, N₃, CN, C(═O)OJ₁, C(═O)NJ₁J₂, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ1J2, and each J₁ and J₂ is independently H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₁-C₆ aminoalkyl, substituted C₁-C₆ aminoalkyl or a protecting group. Exemplary related compounds are disclosed in WO2009006478A. For all chiral centers, asymmetric groups may be found in either R or S orientation.

In some other embodiments, R^{4*} and R^{2*} form the biradical -Q-, wherein Q is C(q₁)(q₂)C(q₃)(q₄), C(q₁)=C(q₃), C[═C(q₁)(q₂)]-C(q₃)(q₄) or C(q₁)(q₂)-C[═C(q₃)(q₄)]; q₁, q₂, q₃, q₄ are each independently H, halogen, C₁₋₁₂ alkyl, substituted C₁₋₁₂ alkyl, C₂₋₁₂ alkenyl, substituted C₁₋₁₂ alkoxy, OJ₁, SJ₁, SOJ₁, SC₂J₁, NJ₁J₂, N₃, CN, C(═O)OJ₁, C(═O)—NJ₁J₂, C(═O) J₁, —C(═O)NJ₁J₂, N(H)C(═NH)NJ₁J₂, N(H)C(═O)NJ₁J₂ or N(H)C(═S)NJ₁J₂, each J₁ and J₂ are independently H, C₁₋₆ alkyl, C₂₋₆ alkenyl, C₂₋₆ alkynyl, C₁₋₆ aminoalkyl or a protecting group, and optionally, wherein when Q is C(q₁)(q₂)(q₃)(q₄) and one of q₃ or q₄ is CH₃, then at least one of the other of q₃ or q₄ or one of q₁ and q₂ is other than H. In some embodiments, R^{1*}, R², R³, R⁵, R^{5*} are hydrogen. Exemplary bicyclic nucleotides are disclosed in WO2008/154401. In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1*}, R², R³, R⁵, R^{5*} are hydrogen. In some embodiments, R^{1*}, R², R³ are hydrogen and one or both of R⁵, R^{5*} may be other than hydrogen as referred to above and in WO 2007/134181 or WO2009/067647 (disclosing certain alpha-L-bicyclic nucleic acids analogs), each of which is incorporated by reference in its entirety. For all chiral centers, asymmetric groups may be found in either R or S orientation.

In some embodiments, LNA monomers have a structure selected from the following group (from the left, formula II, formula III, and formula IV, respectively):

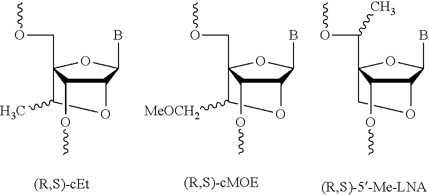

In certain embodiments of the foregoing structures, B is a nucleobase, including nucleobase analogues and naturally occurring nucleobases, such as a purine or pyrimidine, or a substituted purine or substituted pyrimidine. In certain non-limiting embodiments B can be a nucleobase selected from the group consisting of adenine, cytosine, thymine, adenine, uracil, or a modified or substituted nucleobase, such as 5-thiazolo-uracil, 2-thio-uracil, 5-propynyl-uracil, 2-thio-thymine, 5-methylcytosine, 5-thiozolo-cytosine, 5-propynyl-cytosine, and 2,6-diaminopurine. For all chiral centers, asymmetric groups may be found in either R or S orientation.

Certain additional bicyclic nucleoside analogues suitable for use in monomers of the present disclosure are disclosed in WO 2011/115818, WO 2011/085102, WO 2011/017521, WO 2009/100320, WO 2010/036698, WO 2009/124295 and WO 2009/006478.

In some embodiments, LNA monomers have the following structure (formula V):

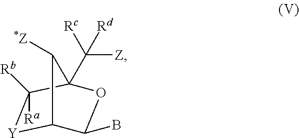

wherein Y is selected from the group consisting of —O—, —CH₂O—, —S—, —NH—, N(R^{e}) and —CH₂—;
Z and Z* are independently selected from the group consisting of an internucleotide linkage, R^{H}, a terminal group and a protecting group, wherein R^{H} is selected from hydrogen and C₁₋₄-alkyl;
B is a natural or non-natural nucleobase; and
R^{a}, R^{b}R^{c}, R^{d} and R^{e} are independently selected from the group consisting of hydrogen, substituted C₁₋₁₂-alkyl, substituted C₂₋₁₂-alkenyl, substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents, R^{a} and R^{b}, together may designate a substituted methylene (═CH₂). In some embodiments R^{a}, R^{b}R^{c}, R^{d} and R^{e} are independently selected from the group consisting of hydrogen and C₁₋₆ alkyl, such as methyl.

For all chiral centers, asymmetric groups may be found in either R or S orientation.

Two exemplary stereochemical isomers of formula V include the following beta-D and alpha-L configurations (from the left, formula VI and formula VII, respectively),

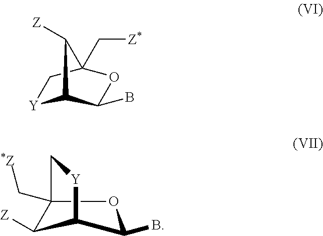

specific exemplary embodiments of which are shown below (formula VIII, IX, X, XI, XII, respectively):

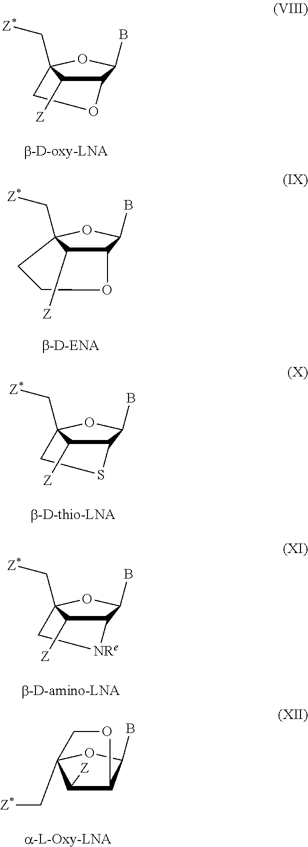

For formulae VI, VII, VIII, IX, X, XI, and XII, the designations for groups B, Y, Z, Z* are the same as those described for formula V, above.

As used herein, the term “thio-LNA” refers to an LNA monomer in which Y in formula VI or formula VII above is either —S— or —CH₂—S—. A thio-LNA monomer can be in either the beta-D or alpha-L-configuration.

As used herein, the term “amino-LNA” refers to an LNA monomer in which Y in formula VI or formula VII above is selected from —N(H)—, N(R)—, CH₂—N(H)—, and —CH₂—N(R)—, where R is selected from hydrogen and C₁₋₄-alkyl. An amino-LNA monomer can be in either the beta-D or the alpha-L-configuration. The term “C₁₋₄-alkyl” means a linear or branched saturated hydrocarbon chain wherein the chain has from one to four carbon atoms, for example, methyl, ethyl, n-propyl, isopropyl, n-butyl, isobutyl, sec-butyl and tert-butyl.

As used herein, the term “oxy-LNA” refers to an LNA monomer in which Y in formula VI or formula VII above is either —O— or —CH₂—O—. An oxy-LNA monomer can be in either the beta-D or the alpha-L-configuration.

As used herein, the term “ENA” refers to an LNA monomer in which Y in formula VI or formula VII above is —CH₂—O— (where the oxygen atom of —CH₂—O— is attached to the 2′-position relative to the base B).

In certain embodiments, the LNA monomer is selected from a beta-D-oxy-LNA monomer, an alpha-L-oxy-LNA monomer, a beta-D-amino-LNA monomer and a beta-D-thio-LNA monomer, in particular a beta-D-oxy-LNA monomer.

### RNAse H Recruitment

Without wishing to be bound by any particular theory of operation, according to certain embodiments, oligomers of the disclosure are capable of recruiting an endoribonuclease, such as Ribonuclease H (RNase H), to RNA targets to which the oligomers hybridize. RNase H is an endoribonuclease that specifically hydrolyzes the phosphodiester bonds of RNA that is hybridized to DNA. Thus, after recruitment, RNase H operates to hydrolyze and eliminate the RNA target, such as a targeted mRNA. Other mechanisms of action are possible. For example, certain oligomers of the disclosure may also be capable of preventing translation of target mRNAs to which the oligomers have hybridized through steric hindrance of the translation machinery.

In certain embodiments, an oligomer comprises a region of at least 6, 7, 8, 9, 10, 11, 12, 13, 14, 15 or 16 contiguous monomers which, after forming a duplex with a target region of a target RNA, contributes to recruiting RNase, such as RNase H. The oligomer region capable of recruiting RNAse may be region B, as that term is defined elsewhere herein. In some embodiments, the region of the oligomer capable of recruiting RNAse H, e.g., region B, consists of 6, 7, 8, 9, 10, 11, 12, 13, 14, 15, 16, 17, 18, 19 or 20 monomers.

US 2004-0102618 A1 provides exemplary, non-limiting, in vitro methods for measuring RNaseH activity that may be used to determine if oligomers of the disclosure can recruit RNaseH. Using these methods, e.g., such as those described in Examples 91-95 of US 2004-0102618, an oligomer is deemed capable of recruiting RNase H if the initial rate of RNase H activity (pmol/L/min) against a duplex containing an RNA target and complementary test oligomer is at least about 1%, 5%, 10%, 20%, 30%, 40%, 50%, 60%, 70%, 80%, 90% or more of the RNase H activity against the same RNA target bound by an oligonucleotide containing the same base sequence, but in which all monomers are DNA, there are no 2′ substitutions, and all internucleoside linkages are phosphorothioate.

In some embodiments, the region of the oligomer responsible for recruiting RNase H after forming a duplex with a target RNA comprises or consists of DNA monomers only, or comprises in addition LNA monomers. In certain embodiments, the LNA monomers are in the alpha-L configuration, a non-limiting example of which is alpha-L-oxy LNA.

An oligomer of the disclosure may comprise nucleosides and nucleoside analogues, each alone or in combination, and may be a gapmer, a headmer, a mixmer (as those terms are defined herein), or some other configuration.

A “headmer” is defined as an oligomer that comprises a first region and a second region of contiguously linked monomers, where the 5′-most monomer of the second region is linked to the 3′-most monomer of the first region, the first region comprising a contiguous stretch of linked nucleoside analogues relatively incapable of recruiting RNase H and the second region comprising a contiguous stretch of linked monomers, for example, DNA monomers, capable of recruiting RNase H after hybridizing to a target RNA.

A “tailmer” is defined as an oligomer that comprises a first region and a second region of contiguously linked monomers, where the 5′-most monomer of the second region is linked to the 3′-most monomer of the first region, the first region comprising a contiguous stretch of linked monomers, for example, DNA monomers, capable of recruiting RNase H after hybridizing to a target RNA and the second region comprising a contiguous stretch of linked nucleoside analogues relatively incapable of recruiting RNase H.

A “mixmer” is an oligomer comprising contiguously linked monomers of both types, i.e., monomers that are capable of recruiting RNase H (if not alone, then contiguously linked with similar monomers), e.g., DNA monomers, and monomers relatively incapable of recruiting RNase H, where monomers of each type are not segregated into separate regions. In certain embodiments, mixmers comprise monomers of both types in an alternating pattern that may be regular or irregular.

### Gapmer Design

According to certain embodiments, the oligomer of the disclosure is a gapmer. A “gapmer” is an oligomer comprising at least one stretch of contiguously linked monomers capable of recruiting an RNAse (for example, RNAseH) when the oligomer forms a duplex with a complementary RNA molecule (such as a mRNA target).

In some embodiments, monomers capable of recruiting RNAse include DNA monomers, alpha-L-LNA monomers, C4′ alkylayted DNA monomers (as described further in WO/2009/090182 and Vester et al., Bioorg. Med. Chem. Lett. 18 (2008) 2296-2300,), and unlocked nucleic acid (UNA) in which the bond between the ribose C2′ and C3′ atoms is cleaved as described in Fluiter et al., Mol. Biosyst., 5:838-843 (2009).

In some embodiments, a gapmer consists of three regions, A-B-C, contiguously arranged from 5′ to 3′. Region A may also be called a 5′ wing or flanking segment or region, region B may also be called a gap or central segment or region, and region C may also be called a 3′ wing or flanking segment or region.

In some embodiments, region A consists of 1, 2, 3, 4, 5 or 6 contiguously linked nucleoside analogues, such as an affinity-enhancing nucleoside analogue. In some embodiments, region C consists of 1, 2, 3, 4, 5 or 6 contiguously linked nucleoside analogues, such as an affinity-enhancing nucleoside analogue. In some embodiments, regions A and C each consist of 1, 2, 3, 4, 5 or 6 contiguously linked nucleoside analogues. In any of the foregoing embodiments, any one or more of the nucleoside analogs of region A and/or region C can be a LNA monomer. In some embodiments, all the monomers of region A and/or region C are LNA monomers.

In some embodiments, region B consists of 1, 2, 3, 4, 5, 6, 7, 8, 9, 10, 11 or 12 contiguously linked monomers capable of recruiting an RNAse, for example RNAse H, when the gapmer forms a duplex with a target RNA, for example mRNA. In some embodiments, any one or more of the monomers of region B is a DNA monomer. In some embodiments, any one or more of the monomers of region B is a nucleotide. In some embodiments, any one or more of the monomers of region B is a DNA nucleotide. In some embodiments all monomers of region B are DNA nucleotides.

In some embodiments, a gapmer optionally includes an additional region D positioned 5′ to region A (that is, D-A-B-C), or positioned 3′ of region C (that is, A-B-C-D). According to such embodiments, regions A and/or C can each independently consist of 1, 2, 3, 4, 5 or 6 contiguously linked nucleoside analogues, any one or more of which can be a LNA monomer, region B consists of 1, 2, 3, 4, 5, 6, 7, 8, 9, 10, 11 or 12 contiguously linked monomers capable of recruiting an RNAse, any one or more of which can be a DNA monomer, such as a DNA nucleotide, and region D consists of 1, 2 or 3 contiguously linked monomers, for example, DNA monomers.

Exemplary gapmers of the disclosure include those having the following structure, where the first position corresponds to the number of contiguously linked nucleoside analogues in region A, the second position corresponds to the number of contiguously linked DNA monomers in region B, and the third position corresponds to the number of contiguously linked nucleoside analogues in region C: 1-8-1, 1-8-2, 2-8-1, 2-8-2, 3-8-3, 2-8-3, 3-8-2, 4-8-1, 4-8-2, 1-8-4, 2-8-4, 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, 3-10-1, 3-10-3, 3-10-4, 4-10-3, 3-9-3, 3-9-4, 4-9-3, 3-8-3, 3-8-4, 4-8-3, 3-7-3, 3-7-4, 4-7-3, 2-7-1, 1-7-2, 2-7-2, 2-7-3, 3-7-2, 5-10-5, 4-12-4. In each of these embodiments, any one or more of the monomers of region A can be a LNA monomer, any one or more of the monomers of region C can be a LNA monomer and any one or more of the monomers of region B can be a DNA monomer. In some embodiments, each monomer of region A and region C are LNA monomers and each monomer of region B is a DNA monomer.

In certain embodiments of the foregoing gapmers, region A and/or region C comprise monomers having a 2′-O-methoxyethyl-ribose sugar (2′ MOE) or monomers having a 2′-fluoro-deoxyribose sugar.

In certain embodiments of the foregoing gapmers, region B comprises at least one LNA monomer in the alpha-L configuration. In certain embodiments, region B comprises at least one alpha-L-oxy LNA monomer. In other embodiments, all the LNA monomers in region B are alpha-L-oxy LNA monomers.

According to some embodiments, a gapmer can be a “shortmer,” defined as a gapmer 10, 11, 12, 13 or 14 contiguously linked monomers in length.

Additional gapmer designs are possible, including those disclosed in WO 2004/046160 and WO 2007/146511.

### Linkage Groups

The monomers of the oligomers, including gapmers, described herein are coupled together into a contiguously linked sequence via linkage groups. Each monomer (except for the monomer occupying the 3′-most position in an oligomer) is linked to a 3′-adjacent monomer via a linkage group.

The terms “linkage group” or “internucleoside linkage” mean a chemical group capable of covalently coupling together two contiguous monomers. Specific non-limiting examples include phosphate groups (forming a phosphodiester between adjacent nucleoside monomers) and phosphorothioate groups (forming a phosphorothioate linkage between adjacent nucleoside monomers).

In certain embodiments, nuclease resistant internucleoside linkages can be used in place of more nuclease sensitive linkages. In a non-limiting example, a phosphorothioate or boranophosphate linkage can be used in place of a phosphodiester linkage group. The former linkage chemistries are more resistant to endo and exonucleases but are still capable of activating RNAse H, thereby permitting RNAse H-mediated antisense inhibition of expression of a target gene.

In some embodiments, linkage groups containing sulphur (S) can be used. As noted above, phosphorothioate linkage groups can be used to link together adjacent monomers of the oligomer, particularly for the gap region (that is, region B) of gapmers. In certain embodiments, phosphorothioate linkages can be used to link together monomers in the flanking A and C regions as well. In other embodiments, phosphorothioate linkages can be used for linking regions A or C to a region D, as well as for linking together monomers within region D if present.

In various embodiments, regions A, B and C comprise linkage groups other than phosphorothioate, such as phosphodiester linkages, particularly, for instance when the use of certain nucleoside analogues acts to protect sensitive linkage groups within regions A and C from endo or exonuclease digestion, for example, when regions A and C comprise LNA monomers.

Inclusion of phosphodiester linkages, for example one, two or three linkages, into an oligomer with a predominantly phosphorothioate backbone, particularly where phosphorothioate groups link nucleoside analogue monomers (for example, in regions A and/or C), can modify the bioavailability and/or bio-distribution of the resulting oligomer. This is described in additional detail in WO2008/113832.

In some embodiments all internucleoside linkage groups are phosphorothioate, whereas in other embodiments the internucleoside linkages of oligomers include both phosphorothioate and phosphodiester linkages.

In describing specific gapmer sequences it should be understood that where phosphorothioate linkages are specified, alternative linkage chemistries disclosed herein, for example phosphodiester linkages, may be used in their place, particularly for linkages between nucleoside analogues, such as LNA monomers.

In another embodiment, oligomers of the disclosure can comprise at least one bicyclic nucleoside attached to the 3′ and/or 5′ terminus by a neutral internucleoside linkage. Use of neutrally linked terminal bicyclic nucleosides is described in additional detail in WO 2009/124238. Examples of neutral internucleoside linkages include phosphotriester, a methylphosphonate, methylene(methylimino) or MMI, amide-3, formacetal or thioformacetal. The remaining linkages in the oligomer may be phosphorothioate or some other type of internucleoside linkage.

### Conjugates

As used herein, the term “conjugate” is intended to indicate a heterogenous molecule formed by the covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, and is hereby incorporated by reference in its entirety.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation (to a conjugate moiety) may be performed in order to enhance or otherwise alter the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example from 1-50, such as 2-20 such as 3-10 amino acid residues in length, and/or polyalkylene oxide such as polyethylglycol(PEG) or polypropylene glycol (see, for example, WO 2008/034123, which is hereby incorporated by reference in its entirety). Suitably the positively charged polymer, such as a polyalkylene oxide may be attached to the oligomer of the invention via a linker such as the releasable inker described in WO 2008/034123.

By way of example, the following conjugate moieties may be used in the conjugates of the invention:

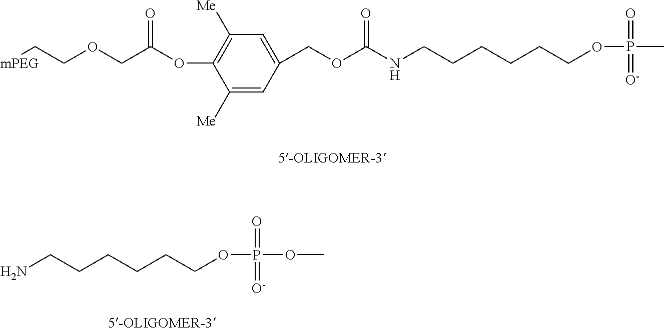

### Activated Oligomers

The term “activated oligomer,” as used herein, refers to an oligomer of the disclosure that is covalently linked to at least one functional moiety that permits covalent linkage of the oligomer to one or more moieties to form a conjugate as described above. Functional moieties are chemical groups that can be covalently bonded to the oligomer.

In some embodiments, a functional moiety can be bonded to an oligomer via a 5′ or 3′ terminal group, or chemical group located elsewhere in an oligomer. Non-limiting examples include amino, sulfhydryl or hydroxyl groups, for example, a 3′-hydroxyl group or an exocyclic NH₂ group of an adenine base. In other embodiments, a functional group can be bonded to a spacer that in turn is bonded to the oligomer.

In some embodiments, terminal and other groups are unprotected, whereas in some other embodiments, the terminal or other group is protected, for example, by any suitable protecting group such as those described in “Protective Groups in Organic Synthesis” by Theodora W Greene and Peter G M Wuts, 3rd edition (John Wiley & Sons, 1999).

Non-limiting examples of hydroxyl protecting groups include esters such as acetate ester, or aralkyl groups such as benzyl, diphenylmethyl, triphenylmethyl, or tetrahydropyranyl groups. Non-limiting examples of amino protecting groups include benzyl, alpha-methylbenzyl, diphenylmethyl, triphenylmethyl, benzyloxycarbonyl, or tert-butoxycarbonyl groups, and acyl groups such as trichloroacetyl or trifluoroacetyl groups.

In some embodiments, the functional moiety is self-cleaving. In other embodiments, the functional moiety is biodegradable. See e.g., U.S. Pat. No. 7,087,229, which is incorporated herein by reference in its entirety.

In some embodiments, oligomers of the disclosure are functionalized at the 5′ end in order to allow covalent attachment of a moiety to the 5′ end of the oligomer. In other embodiments, oligomers of the disclosure can be functionalized at the 3′ end to allow covalent attachment of a moiety to the 3′ end of the oligomer. In still other embodiments, oligomers of the disclosure can be functionalized along the backbone or on the heterocyclic base moiety. In yet other embodiments, oligomers of the disclosure can be functionalized at more than one position independently selected from the 5′ end, the 3′ end, the backbone and the base.

In some embodiments, activated oligomers of the disclosure are synthesized by incorporating during the synthesis one or more monomers that are covalently attached to a functional moiety. In other embodiments, activated oligomers of the disclosure are synthesized with unfunctionalized monomers and the oligomer is functionalized after completion of synthesis.

In some embodiments, the oligomers are functionalized with a hindered ester containing an aminoalkyl linker, wherein the alkyl portion has the formula (CH₂)_{w}, wherein w is an integer ranging from 1 to 10, for example 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, for example 6, wherein the alkyl portion of the alkylamino group can be straight chain or branched chain, and wherein the functional group attached to the oligomer via an ester group (—O—C(O)—(CH₂)_{w}SH).

In some embodiments, sulfhydryl-activated oligonucleotides are conjugated with polymer moieties such as polyethylene glycol or peptides via formation of a disulfide bond.

Activated oligomers covalently linked to at least one functional moiety can be synthesized by any method known in the art, including those disclosed in U.S. Patent Publication No. 2004/0235773 and in Zhao et al. (2007) J. Controlled Release 119:143-152; and Zhao et al. (2005) Bioconjugate Chem. 16:758-766.

In still other embodiments, oligomers of the disclosure can be functionalized by introducing sulfhydryl, amino or hydroxyl groups into the oligomer using substantially linear functionalizing reagents having a phosphoramidite at one end linked through a hydrophilic spacer chain to the opposing end which comprises a protected or unprotected sulfhydryl, amino or hydroxyl group. Such reagents, their synthesis and use to functionalize monomers or oligomers are described further in U.S. Pat. Nos. 4,962,029 and 4,914,210, each of which is incorporated herein by reference in its entirety. These reagents primarily react with hydroxyl groups of oligomers. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the disclosure have a functionalizing reagent coupled to a hydroxyl group on the backbone of the oligomer.

In some embodiments, the 5′-terminus of an oligomer bound to a solid-phase is functionalized with a dienyl phosphoramidite derivative followed by conjugation of the deprotected oligomer with, e.g., an amino acid or peptide via a Diels-Alder cycloaddition reaction.

In various embodiments, incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 32:7171 (1991).

In still further embodiments, oligomers of the disclosure can be provided with amine-containing functional moieties on the nucleobase, including on the N6 purine amino groups, on the exocyclic N2 of guanine, or on the N4 or 5 positions of cytosine. In some embodiments, such functionalization may be achieved during oligomer synthesis by using a commercial reagent that is already functionalized.

Certain functional moieties are available from commercial sources. For example, heterobifunctional and homobifunctional linking moieties are available from the Pierce Co. (Rockford, Ill.), 5′-Amino-Modifier C6 reagents are available from Glen Research Corporation (Sterling, Va.) and from ABI (Applied Biosystems Inc., Foster City, Calif.) as Aminolink-2, and 3′-Amino-Modifier reagents are available from Glen Research Corporation (Sterling, Va.) and from Clontech Laboratories Inc. (Palo Alto, Calif.).

### Pharmaceutical Compositions Comprising Oligomers

The disclosure further provides pharmaceutical compositions comprising oligomers or conjugates.

Oligomers of the disclosure may be admixed with pharmaceutically acceptable active and/or inert substances for the preparation of pharmaceutical compositions or formulations. Pharmaceutical compositions of the disclosure include, but are not limited to, solutions, suspensions, emulsions, and liposome-containing formulations.

Suspensions can be prepared using aqueous, non-aqueous or mixed media. Aqueous suspensions may further contain substances which increase the viscosity of the suspension including, for example, carboxymethylcellulose, sorbitol or dextran.

Compositions of the disclosure can also include a pharmaceutically acceptable carrier diluent or solvent, such as sterile water, alcohol, saline or phosphate-buffered saline (PBS). Such diluents are useful, for example, in compositions to be delivered parenterally, for example, intrathecally or intravenously.

Compositions of the disclosure can also include one or more pharmaceutically acceptable excipients. Excipients are selected so as to provide for certain desired properties of the composition, such as stability, bioavailability, or pharmacokinetics of the active agent, and ease of administration according to the desired mode. See, e.g., Martin, Remington's Pharmaceutical Sciences, 18th Ed. (1990, Mack Publishing Co., Easton, Pa. 18042). Compositions may be prepared in liquid form, or may be in dried powder, such as lyophilized form.

Exemplary pharmaceutical excipients include, but are not limited to, carrier substances, such as polyinosinic acid, dextran sulfate, polycytidic acid or 4-acetamido-4′ isothiocyano-stilbene-2,2′-disulfonic acid, fillers, bulking substances, such as an inert bulk nucleic acid, or stabilizers, such as lactose, sucrose, mannitol and other sugars, pH buffers, such as Tris-HCl, acetate, or phosphate, detergents, surfactants, including ionic and non-ionic surfactants, such as polysorbate 80, solubilizing agents, wetting agents, anti-oxidants, such as ascorbic acid or sodium metabisulfite, preservatives, such as thimersol or benzyl alcohol, and chelating agents.

In other embodiments, compositions can include agents that enhance uptake of oligonucleotides at the cellular level. For example, cationic lipids, such as lipofectin (Junichi et al, U.S. Pat. No. 5,705,188), cationic glycerol derivatives, and polycationic molecules, such as polylysine (Dello et al., PCT Application WO 1997/30731), have been demonstrated to enhance the cellular uptake of oligonucleotides. Other agents that can be added to enhance the penetration of oligomers include glycols such as ethylene glycol and propylene glycol, pyrrols such as 2-pyrrol, azones, and terpenes such as limonene and menthone.

To prepare pharmaceutical compositions, oligomers of the disclosure can be used in unmodified form, or can be modified to create prodrugs, pharmaceutically acceptable acid and base addition salts, esters, or salts of such esters, of such oligomers. See, for example, Berge, et al., “Pharmaceutical Salts,” J. of Pharm. Sci., 1977, 66, 1-19.

A prodrug can include the incorporation of additional nucleosides or nucleotides at one or both ends of an oligomer that are cleaveable by endogenous nucleases within a subject's body to form the biologically active oligomer. Prodrug versions of oligomers of the disclosure can be prepared as SATE [(S-acetyl-2-thioethyl)phosphate]derivatives according to the methods disclosed in WO 1993/24510, WO 1994/26764 and U.S. Pat. No. 5,770,713.

As used herein, the term “pharmaceutically acceptable salt” refers to a salt that retains the desired biological activity of the oligomer while at the same time exhibiting acceptable levels of undesired toxic effects.

Pharmaceutically acceptable base addition salts of the oligomers of the disclosure can be formed with basic compounds, such as metal cations, such as alkali and alkaline earth metals, ammonium and quaternary ammonium cations, or amines, such as organic amines. Non-limiting examples of metal cations include sodium, potassium, magnesium, calcium, zinc, bismuth, barium, aluminum, copper, cobalt, nickel, or cadmium. Non-limiting examples of organic amines are N,N′-dibenzylethylenediamine, chloroprocaine, choline, diethanolamine, dicyclohexylamine, ethylenediamine, N-methylglucamine, D-glucosamine, tetraethylammonium, and procaine. Polyamines such as spermine and spermidine can also be used. Base addition salts can be prepared by contacting the free acid form of a compound, for example, an oligomer, with a sufficient amount of the desired base to produce the salt. The free acid form may be regenerated by contacting the salt form with an acid and isolating the free acid.

Pharmaceutically acceptable acid addition salts of the oligomers of the disclosure can be formed with organic or inorganic acids. Exemplary inorganic acids include hydrochloric acid, hydrobromic acid, sulfuric acid, phosphoric acid, or nitric acid. Exemplary organic acids include carboxylic, sulfonic, sulfo or phospho acids, or N-substituted sulfamic acids. More specific examples include acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, tannic acid, palmitic acid, alginic acid, polyglutamic acid, methanesulfonic acid, 4-aminosalicylic acid, 2-phenoxybenzoic acid, 2-acetoxybenzoic acid, embonic acid, nicotinic acid, isonicotinic acid, phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalenesulfonic acid, naphthalenedisulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), p-toluenesulfonic acid, polygalacturonic acid, or ascorbic acid. Other organic acids include any of the natural or non-naturally occurring amino acids, including for example, glutamic acid or aspartic acid.

Pharmaceutically acceptable salts can also be formed from elemental anions such as chlorine, bromine, and iodine.

In some embodiments, compositions comprising oligomers of the disclosure can be administered together with at least a second agent to treat diseases caused by expansion of the C9ORF72 hexameric repeat region. In some embodiments, the second agent is a second oligomer of the disclosure which can be included in the same or a different composition as the first oligomer of the disclosure.

Pharmaceutical compositions may be prepared in unit dosage form for the convenience of care providers and to help ensure sterility and reduce wastage. Such dosage forms can be included in kits that additionally provide a device for administering the composition, such as a syringe and needle for intravenous or intrathecal delivery. In some embodiments, the pharmaceutical composition comprising an oligomer of the disclosure is provided in liquid form, and in other embodiments is provided in lyophilized form. When a lyophilized preparation of the composition is provided, a kit can additionally provide a suitable diluent, such as sterile water, saline, or other diluent.

### Dosages

Optimal dosing of the oligomers of the disclosure will depend on a variety of factors, including the indication to be treated, the specific biological activity of the oligomer, therapeutic index of the oligomer and the mode of administration. Typically, the chosen dose seeks to maximize efficacy in a particular subject under treatment while minimizing any expected side effects.

In some embodiments, dosage can be expressed as a certain mass of the oligomer to be administered per unit of mass of the subject being treated. Exemplary dosages include 10 microgram (mcg) per kg, 25 mcg/kg, 50 mcg/kg, 75 mcg/kg, 100 mcg/kg, 200 mcg/kg, 300 mcg/kg, 400 mcg/kg, 600 mcg/kg, 700 mcg/kg, 800 mcg/kg, 900 mcg/kg, 250 mcg/kg, 500 mcg/kg, 750 mcg/kg, 1 milligram (mg) per kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 150 mg/kg, 200 mg/kg or more.

According to other embodiments, the mass of oligomer to be administered is not related to mass of the subject, but to some other variable, such as the subject's brain volume or mass, or volume of cerebrospinal fluid. According to yet other embodiments, for example where a composition comprising an oligomer of the disclosure is to be administered intrathecally or intraventricularly, the total mass of oligomer to be administered may be less than if the administration were to be made intravenously or in some other way outside the central nervous system.

### Methods of Treatment or Prevention

According to certain embodiments, the disclosure provides for methods of treating or preventing diseases or disorders of the central nervous system associated with expansion of the hexanucleotide repeat region of the C9ORF72 gene, or any other gene targeted by an oligomer of the disclosure. In some embodiments, the disease is frontotemporal dementia (FTD) or amyotrophic lateral sclerosis (ALS). Methods of treatment are carried out by administering a therapeutically effective amount of an oligomer of the disclosure, or composition thereof, to a subject in thereof diagnosed as having the disease or disorder or who is determined to be at greater than normal risk of later developing the disease or disorder. Methods of diagnosis and determinations as to predisposition for developing a disease or disorder are known in the art, including without limitation, observations of behavior, biopsies or genetic tests.

Methods are also provided for down-regulating the expression of the C9ORF72 gene in a cell or tissue by contacting said cell or tissue with an effective amount of an oligomer of the disclosure, or composition comprising such oligomer. Exemplary cells include neurons and exemplary tissues include brain tissue, for example, gray matter (i.e., that portion of brain tissue predominantly comprising the cell bodies of neurons).

In other embodiments, the disclosure provides for the use of the oligomers of the disclosure for the manufacture of a medicament for the treatment or prevention of a disease or disorder of the central nervous system caused by expansion of the hexanucleotide repeat region of the C9ORF72 gene, or any other gene targeted by an oligomer of the disclosure. In some embodiments, the disease is frontotemporal dementia (FTD) or amyotrophic lateral sclerosis (ALS).

According to some embodiments, the subject to be treated is a human, and in other embodiments, the subject is a non-human animal, for example, non-human mammal.

As used herein, “treatment” refers both to treatment of a disease associated with hexanucleotide repeat expansion where symptoms have already presented, as well as prevention of such disease in susceptible subjects who have not experienced a prodrome or more severe symptoms. Thus, in certain embodiments, treatment includes prevention or prophylaxis.

In some embodiments, the disease to be treated with an oligomer of the disclosure is frontotemporal dementia (FTD) or amyotrophic lateral sclerosis (ALS) associated with hexanucleotide repeat expansion. The diseases that may be treated with the instant oligomers are not limited to FTD and ALS, however. Instead, any disease associated with a hexanucleotide repeat expansion that can be targeted with an oligomer of the disclosure can be treated, even if such diseases have not yet been recognized in the art.

Treatment involves administering a therapeutically effective amount of an oligomer of the disclosure, or composition thereof. Therapeutic efficacy means any temporary or permanent reduction or amelioration in the severity of symptoms associated with the disease under treatment, or improvement in the condition, functioning or prognosis of the subject with the disease being treated. Therapeutic efficacy can also refer to a reduction in the rate at which a progressive disease worsens, a delay of onset of the first symptoms associated with a disease, or delay before a subject with a lethal disease ultimately succumbs and dies, compared to untreated subjects with the disease.

In the case of ALS, administration of a therapeutically effective amount of an oligomer of the disclosure can reduce muscle weakness associated with ALS, such as in the hands, arms or legs, or in the muscles that control speech, swallowing or breathing. Efficacious treatment can also improve the ability of the subject with ALS to form speech, swallow or breathe, or use his or her arms or legs. Efficacious treatment can also improve mobility, as well as reduce twitching or fasciculation and cramping of the muscles, especially in the hands and feet. Therapeutic efficacy is also associated with a reduction or cessation in the rate at which motor neurons affected by the disease die. Methods for determining amelioration of disease symptoms, including those described herein, may be assessed using methods well-known in the art.

In the case of FTD, symptoms that may improve after administration of therapeutically effective amount of an oligomer depends in part on the type of FTD a subject has. For example, with behavioral variant frontotemporal dementia (bvFTD), the most significant initial symptoms are associated with personality and behavior. Thus, in the case of bvFTD, a therapeutically effective amount of an oligomer can reduce the extent or rate at which a subject experiences disinhibition, which presents as a loss of restraint in personal relations and social life, as assessed according to methods well-known in the art.

With primary progressive aphasia (PPA), the most significant initial symptoms affects language skills. In semantic dementia, subjects can still form language, but their words convey less and less meaning as they substitute more general for specific terms (e.g., animal instead of cat). Language comprehension also declines. In progressive nonfluent aphasia, subjects lose their ability to generate words easily and speech becomes halting and ungrammatical. Ability to read and write also may be impaired. Thus, in the case of this type of FTD, a therapeutically effective amount of an oligomer can reduce the extent or rate at which the disease process impairs language formation or the ability to read and write. Language functioning can be assessed with certain standardized tests and/or other methods well-known in the art.

In some subjects, FTD initially presents as a movement disorder affecting certain involuntary, automatic muscle functions. These disorders also may impair language and behavior. The two primary FTD movement disorders are corticobasal degeneration (CBD), which causes shakiness, lack of coordination and muscle rigidity and spasms, and progressive supranuclear palsy (PSP), which causes walking and balance problems, frequent falls and muscle stiffness, especially in the neck and upper body, as well as affecting eye movements. In the case of this type of FTD, a therapeutically effective amount of an oligomer can reduce the extent or rate at which the disease process impairs muscle function which may be assessed by methods well-known in the art.

In other embodiments, a therapeutically effective amount of an oligomer reduces the extent or rate of neurodegeneration caused by FTD or other neurological disorder associated with hexanucleotide repeat expansion. In addition to an improvement, or at least reduction in the extent or rate of deterioration, in behavioral symptoms, therapeutic efficacy can also be monitored with brain scans, e.g., CAT scan, functional MRI, or PET scan, or other methods well-known in the art.

The therapeutic efficacy of an oligomer of the disclosure can also be predicted based on in vitro tests of the ability of the oligomer to reduce the transcription, transcript stability, or translation of a targeted gene. Efficacy can further be assessed by studying the effect of an oligomer on the cells in and behaviors of animal models for a human disease to be treated using methods well-known in the art.

For the instant methods of treatment or prevention, a therapeutically effective amount of an oligomer of the disclosure, or composition thereof, can be administered as a single dose, or as two or more divided doses separated by predetermined intervals. In non-limiting examples, such intervals include a period of minutes, for example, 5, 10, 15, 30 or more minutes, a period of hours, for example, 1, 1.5, 2, 3, 4, 5, 6, 7, 8, 9, 10, 12, 15, 18, 20 hours or more, a period of days, for example, 1, 1.5, 2, 3, 4, 5, 6 days or more, or a period of weeks, for example, 1, 2, 3, or more weeks.

The invention further provides for an oligomer according to the invention, for use in medicine. In particular, the invention further provides for the use of the oligomer of the invention in the manufacture of a medicament for the treatment of one or more of the diseases referred to herein, such as a disease selected from the group consisting of frontotemporal dementia (FTD) or amyotrophic lateral sclerosis (ALS).

The invention further provides for an oligomer according to the invention, for use for the treatment of one or more of the diseases referred to herein, such as a disease selected from the group consisting of frontotemporal dementia (FTD) or amyotrophic lateral sclerosis (ALS).

### Modes of Administration

Oligomers of the disclosure can be administered by any mode demonstrated to result in the direct or indirect delivery of a therapeutically effective concentration of oligomers into the brains of subjects to be treated. In some embodiments, administration of a composition comprising an oligomer of the disclosure includes parenteral administration, examples of which include administration by the intravenous, intra-arterial, intraperitoneal, intramuscular, subcutaneous, intrathecal or intraventricular routes of administration. Administration by any one or more of these routes can be accomplished by injection or infusion through a needle, catheter or cannula, or by some other means.

In some embodiments, oligomers of the disclosure can be administered intrathecally into the cerebrospinal fluid (CSF) by injecting or infusing a composition containing the oligomer under the arachnoid membrane of the brain or spinal cord. In other embodiments, oligomers of the disclosure can be administered into the CSF by intracerebral ventricular (ICV) administration (or simply intraventricular administration) by injecting or infusing a composition containing the oligomer into the brain ventricles through a catheter or cannula. In some embodiments, the cannula can be connected to an Ommaya reservoir or osmotic pump implanted under the skin of a subject so that oligomers targeting C9ORF72 can be intermittently or continuously infused into the ventricles and CSF over an extended period time, for example, hours, days or weeks. According to various embodiments, the cannula can be directed into the left or right lateral ventricle, the third ventricle or to the fourth ventricle.

ICV has been demonstrated to be an effective way of administering antisense compounds into the brains of test animals. It was demonstrated, for example, that an antisense oligonucleotide targeting superoxide dismutase 1 (SOD1) administered into the ventricles of rats via a cannula reduced both SOD1 mRNA and protein expression throughout the brain and spinal cord. And, in a more recent study, intraventricular administration of an antisense oligonucleotide into mice targeting cellular prion protein was found to delay onset of neurodegenerative disease caused by a pathogenic prion isoform. Smith, et al., J. Clin. Invest. 116:2290-2296 (2006) and Friberg, et al., Molecular Therapy-Nucleic Acids, e9:1-12 (2012), respectively. Thus, in some embodiments, oligomers of the disclosure can be administered directly into the brain ventricles to mix with the cerebrospinal fluid to be transported around the brain and spinal cord and into the parenchyma of these tissues. Oligomers can then be taken up by neurons or other affected cells to target C9ORF72 RNA.

Reports in the scientific literature suggest that at least on some cases, antisense oligonucleotides administered intravenously are able to cross the blood-brain-barrier and affect gene expression within the central nervous system. See, e.g., Farr et al., J Alzheimers Dis. 2014 Jan. 1; 40(4):1005-16, and Farr et al., Free Radic Biol Med. 2014 February; 67:387-95.

### EXAMPLES

### Example 1

### Treatment with oligomers targeting C9ORF72 RNA

Materials and Methods:

Tissue Culture

Cells were cultured in the appropriate medium as described below and maintained at 37° C. at 95-98% humidity and 5% CO₂. Cells were routinely passaged 1-2 times weekly.

ND40063:

Primary human skin fibroblast cells isolated from a 41 year old female subject with familial FTD/ALS contains a C9ORF72 gene with expanded hexanucleotide repeats. ND40063 cells were purchased from Coriell Institute for Medical Research. and cultured in DMEM (Sigma) with 15% FBS+100 units/ml penicillin (Gibco)+100 μg/ml streptomycin (Gibco).

HEK-293:

Human embryonic kidney cell line purchased from ATCC and cultured in DMEM (Sigma) with 10% FBS+100 units/ml penicillin (Gibco)+100 μg/ml streptomycin (Gibco).

Unassisted Oligomer Uptake in Cells (“Gymnotic Delivery”)

Cell culturing: ND40063 or HEK-293 cells were seeded in 24-well plates at 37° C. (5% CO₂) in 0.5 ml growth medium supplemented with 15% or 10% FBS, 100 units/ml penicillin (Gibco) and 100 μg/ml streptomycin (Gibco). Seeding density was 40000 cells/well. Cells were treated in triplicates with different concentrations of oligomers (0.05 μM-50 μM) for 3 days. In short, 25 μl (20×) oligomer was added to a total volume of 0.5 ml cell mix per well. Cells were incubated at 37° C. for 3 days, then harvested for RNA analysis.

Total RNA Isolation

Total RNA was isolated using the RNeasy kit (Qiagen). Cells were washed with PBS, and cell lysis buffer (RTL, Qiagen) supplemented with 1% mercaptoethanol was added directly to the wells. After a few minutes the samples were processed according to manufacturer's instructions. Total RNA was DNAse-I treated before elution from columns to eliminate contaminating genomic DNA.

First Strand Synthesis

First strand synthesis was performed using either random DNA decamers (for total cDNA) or strand-specific primers (sense RT#2 (CAATTCCACCAGTCGCTAGA (SEQ ID NO:177)) or antisense RT#2 (CTGCGGTTGCGGTGCCTGC (SEQ ID NO:178)) together with M-MLV-Reverse Transcriptase (essentially as described by manufacturer (Ambion)). For each reaction, 0.25 μg (total cDNA)/25 ng (strand-specific cDNA) DNase-I-treated total RNA (10.8 μl), 2 μl decamer, 2 μl dNTP mix (2.5 mM each) was mixed. Samples were incubated at 70° C. for 3 min and put on ice to cool. Then 3.25 μl of a mix containing (2 μl 10×RT buffer, 1 μl M-MLV Reverse transcriptase and 0.25 μl RNAse inhibitor) was added. cDNA was synthesized at 42° C. for 60 min followed by heat-inactivation at 95° C. for 10 min.

Real-Time Quantitative PCR Analysis of C9orf72 RNA Levels

To determine the relative human C9orf72 RNA level in treated and untreated samples, the generated cDNA was used in quantitative PCR analysis using a Real-time PCR system from Applied Biosystems. The C9orf72 RNA expression quantified generally as described by the manufacturer. In brief, 4 μl of cDNA was added 6 μl of a mastermix containing Taqman Fast Universal PCR master mix and a primer-probe mix available from Life technologies. All samples were run in duplicates and correlated to a 2-fold dilution series generated from cDNA made from the respective cell line. Relative quantities of C9orf72 RNA were determined from the calculated threshold cycle using the sequence detection software from Applied Biosystems and normalized to the relative quantities of either GAPDH mRNA (qper-assay#1) or an internal Mock sample (qper-assay#2).

Primers used in the quantitative PCR assays were as follows:

Qpcr-assay#1, forward primer:
(SEQ ID NO: 179):
ATCATTTGGGGTTTTGATGG
Qpcr-assay#1, reverse primer:
(SEQ ID NO: 180)
TCTTGGCAACAGCTGGAGAT
Qpcr-assay#1, detection probe:
(SEQ ID NO: 181)
GTTGGAATGCAGTGATGTCG
Qpcr-assay#2, forward primer:
(SEQ ID NO: 182)
AAGAGGCGCGGGTAGAAG
Qpcr-assay#2, reverse primer:
(SEQ ID NO: 183)
AGTCGCTAGAGGCGAAAGC
Qpcr-assay#2, detection probe:
(SEQ ID NO: 184)
CCCTCTCATTTCTCTGACCG

In accordance to the present disclosure, a series of oligomers were designed to target different regions of human C9orf72 RNA. See Table 1: Oligomers are evaluated for their potential to knockdown C9orf72 pre-mRNA in human ND40063 cells following unassisted uptake. The results showed very potent down regulation of total C9orf72 pre-mRNA (75-80%: ASO-13, ASO-146 and ASO-156) with 15.8 μM of all oligomers.

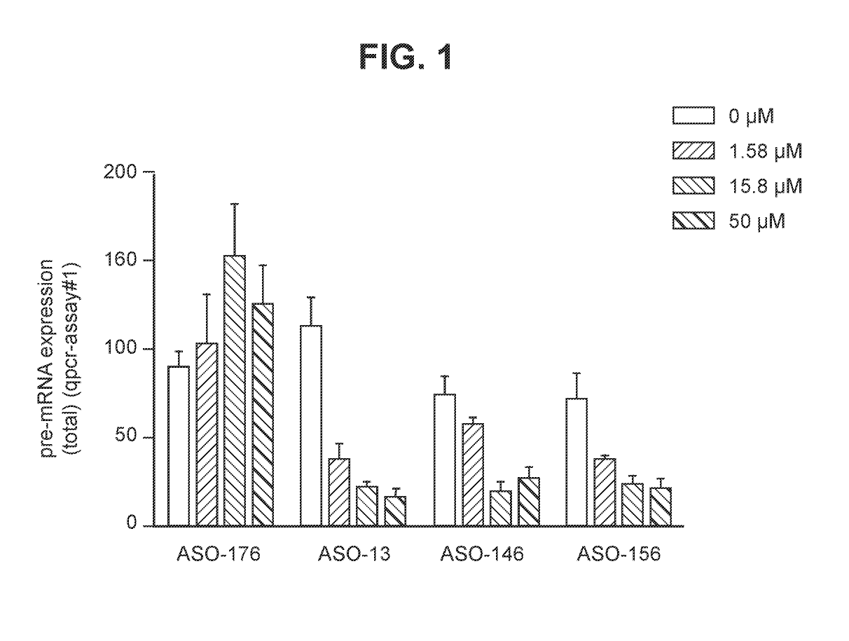

The structure and nucleobase sequence of the oligomers described in FIG. 1 and Table 1 are disclosed in Table 4 in which the oligomer numbers in the first column correspond to the numbers following the prefix “ASO-”.

Human ND40063 cells were treated with different oligomers targeting the C9orf72-repeat expansion. Total C9orf72 pre-mRNA expression after 3 days of oligomer treatment was measured by qPCR using qper-assay#1, normalized to the house keeping gene GAPDH and presented relative to the Mock control. Oligomers were tested at a concentration of 0 μM, 1.58 μM, 15.8 μM and 50 μM. ASO-176 was included as a non-functional control oligomer. Values are represented as Mean+SEM. The results are shown in the graph of FIG. 1.

TABLE 1
Data values presented in FIG. 1.

| Oligomer | 0 μM (N = 3) | | 1.58 μM (N = 3) | | 15.8 μM (N = 3) | | 50 μM (N = 3) | |
|---|---|---|---|---|---|---|---|---|
| ID | Mean | SEM | Mean | SEM | Mean | SEM | Mean | SEM |
| ASO-176 | 90.00 | 9.00 | 103.67 | 27.06 | 152.33 | 29.73 | 125.00 | 22.34 |
| ASO-13 | 112.94 | 16.46 | 37.82 | 9.20 | 22.37 | 2.13 | 16.99 | 4.59 |
| ASO-146 | 74.30 | 10.31 | 57.78 | 4.15 | 20.72 | 4.34 | 27.35 | 6.47 |
| ASO-156 | 72.24 | 13.80 | 37.55 | 2.40 | 24.61 | 3.90 | 21.78 | 5.64 |

### Example 2

### Targeting Both Sense and Antisense C9orf72 RNA Strands with Oligomers

In accordance to the present disclosure, a series of oligomers were tested for their ability to simultaneously target both sense and antisense C9orf72 pre-mRNA strands. See Table 2: Oligomers are evaluated for their potential to knockdown sense or antisense C9orf72 pre-mRNA in human ND40063 cells following unassisted uptake. The results showed very potent down regulation of sense C9orf72 pre-mRNA (80-86%: ASO-9, ASO-154, ASO-156 and ASO-158) and antisense C9orf72 pre-mRNA (76-91%: ASO-9, ASO-154, ASO-156 and ASO-158) with 15.8 μM of all oligomers.

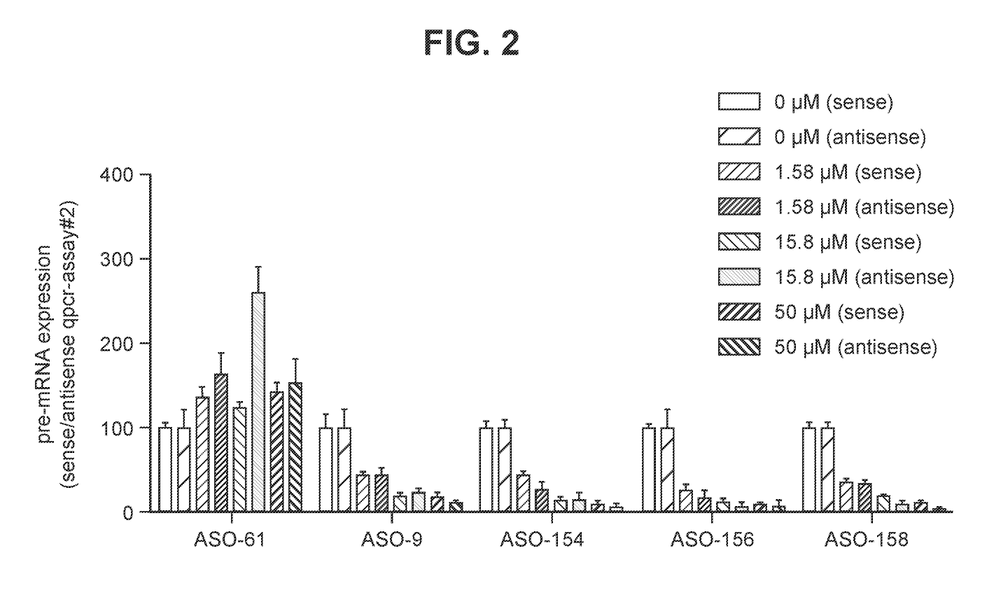

The structure and nucleobase sequence of the oligomers described in FIG. 2 and Table 2 are disclosed in Table 4 in which the oligomer numbers in the first column correspond to the numbers following the prefix “ASO-”.

Human ND40063 cells were treated with different oligomers that target the C9orf72-repeat expansion. After 3 days of oligomer treatment the level of sense and antisense C9orf72 pre-mRNA expression was measured using a strand-specific RT reaction (using either sense RT#2 or antisense RT#2 primer) of 25 ng total RNA combined with qPCR analysis (qper-assay#2). Expression was normalized to the Mock control. ASO-61 was included as a non-functional control oligomer. Values are represented as Mean+SEM. The results are shown in the graph of FIG. 2.

TABLE 2
Data values presented in FIG. 2.

| Oligomer | 0 μM (N = 3) | | 1.58 μM (N = 3) | | 15.8 μM (N = 3) | | 50 μM (N = 3) | |
|---|---|---|---|---|---|---|---|---|
| ID | Mean | SEM | Mean | SEM | Mean | SEM | Mean | SEM |
| Sense | | | | | | | | |
| RT | | | | | | | | |
| ASO-61 | 100.00 | 4.58 | 137.00 | 10.60 | 125.33 | 3.38 | 143.67 | 7.84 |
| ASO-9 | 100.00 | 14.42 | 44.33 | 2.40 | 19.33 | 1.76 | 19.00 | 3.61 |
| ASO-154 | 100.00 | 6.65 | 45.90 | 1.51 | 15.63 | 0.86 | 10.80 | 0.57 |
| ASO-156 | 100.00 | 3.53 | 27.73 | 4.59 | 13.26 | 1.86 | 8.48 | 1.58 |
| ASO-158 | 100.00 | 5.48 | 37.40 | 1.72 | 19.50 | 1.28 | 11.93 | 1.04 |
| Antisense | | | | | | | | |
| RT | | | | | | | | |
| ASO-61 | 100.33 | 19.68 | 164.67 | 22.93 | 260.67 | 28.20 | 152.67 | 27.33 |
| ASO-9 | 100.00 | 20.23 | 44.33 | 7.97 | 23.67 | 3.18 | 11.50 | 1.50 |
| ASO-154 | 100.00 | 9.27 | 27.77 | 7.22 | 15.20 | 6.40 | 5.35 | 3.55 |
| ASO-156 | 100.00 | 20.97 | 18.07 | 6.49 | 8.23 | 2.29 | 7.03 | 5.34 |
| ASO-158 | 100.00 | 5.16 | 33.51 | 2.64 | 10.29 | 2.58 | 0.92 | 0.19 |

### Example 3

### Potent Knock-Down in Cells Containing the C9orf72-Repeat Expansion

A series of oligomers were tested for their ability to specifically target and to knockdown mutant C9orf72 RNA (C9orf72-repeat expansion containing RNA). Table 3 shows how different oligomers specifically down regulate mutant C9orf72 RNA in ND40063 cells, but not wild-type C9orf72 RNA in HEK-293 cells. The results showed very potent down regulation of C9orf72 pre-mRNA in ND40063 cells (80-87%: ASO-127, ASO-144, ASO-145, ASO-146 and ASO-147) and a much milder effect on C9orf72 mRNA (33-44%: ASO-127, ASO-144, ASO-145, ASO-146 and ASO-147) at 25 μM concentration. There was no C9orf72 RNA knockdown observed using the same concentration of oligomer in HEK-293 cells.

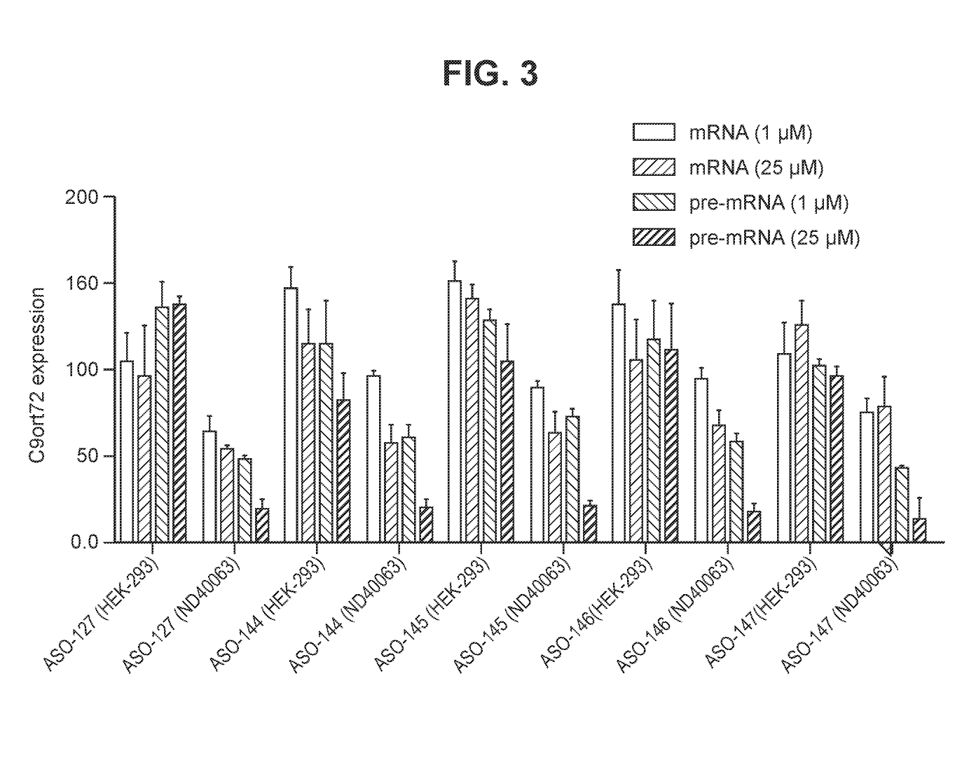

The structure and nucleobase sequence of the oligomers described in FIG. 3 and Table 3 are disclosed in Table 4 in which the oligomer numbers in the first column correspond to the numbers following the prefix “ASO-”.

Human ND40063 and HEK-293 cells were treated with different oligomers that targets the C9orf72-repeat expansion. After 3 days of oligomer treatment the level of C9orf72 pre-mRNA and mRNA expression was measured using quantitative per analysis. All oligomers were found to selectively down regulate C9orf72 pre-mRNA in ND40063 cells with the repeat-expansion, but not in cells without the repeat-expansion (HEK-293). The results are shown in the graph of FIG. 3.

TABLE 3
Data values presented in FIG. 3.

| | | mRNA | | mRNA | | Pre-mRNA | | Pre-mRNA | |
|---|---|---|---|---|---|---|---|---|---|
| | Oligomer | (1 μM, N = 2) | | (25 μM, N = 2) | | (1 μM, N = 2) | | (25 μM, N = 2) | |
| Cell type | ID | Mean | SEM | Mean | SEM | Mean | SEM | Mean | SEM |
| HEK-293 | ASO-127 | 1.04 | 0.16 | 0.96 | 0.29 | 1.36 | 0.14 | 1.38 | 0.03 |
| ND40063 | ASO-127 | 0.64 | 0.08 | 0.54 | 0.01 | 0.48 | 0.01 | 0.19 | 0.05 |
| HEK-293 | ASO-144 | 1.47 | 0.12 | 1.15 | 0.19 | 1.15 | 0.25 | 0.82 | 0.15 |
| ND40063 | ASO-144 | 0.95 | 0.03 | 0.57 | 0.09 | 0.61 | 0.06 | 0.20 | 0.04 |
| HEK-293 | ASO-145 | 1.51 | 0.11 | 1.41 | 0.07 | 1.28 | 0.05 | 1.05 | 0.21 |
| ND40063 | ASO-145 | 0.89 | 0.03 | 0.63 | 0.12 | 0.72 | 0.04 | 0.20 | 0.03 |
| HEK-293 | ASO-146 | 1.37 | 0.19 | 1.05 | 0.22 | 1.17 | 0.21 | 1.11 | 0.27 |
| ND40063 | ASO-146 | 0.95 | 0.06 | 0.67 | 0.09 | 0.58 | 0.04 | 0.18 | 0.03 |
| HEK-293 | ASO-147 | 1.09 | 0.18 | 1.25 | 0.14 | 1.02 | 0.04 | 0.96 | 0.05 |
| ND40063 | ASO-147 | 0.75 | 0.08 | 0.78 | 0.17 | 0.43 | 0.01 | 0.13 | 0.12 |

TABLE 4
Efficacy data for all oligomers tested in ND40063 cells

| Oligo- | Oligomer | | | | Anti- | gDNA | mRNA |
|---|---|---|---|---|---|---|---|
| mer No. | nucleobase | Max. KD | Total | Sense | sense | location | location |
| (SEQ ID | sequence and | efficacy | pre mRNA | pre-mRNA | pre-mRNA | (SEQ ID | (SEQ ID |
| NO:) | structure (5′ → 3′) | (@25 μM) | IC50 (μM) | IC50 (μM) | IC₅₀ (μM) | NO: 187) | NO: 188) |
| 1 | Ggc ccc ggc ccc G | 0.193 | | | | 5326 | N/A |
| 2 | Ccc cgg ccc cgg cC | 0.332 | | | | 5323 | N/A |
| 3 | Gcc ccg gcc ccg gcC | 0.187 | | | | 5323 | N/A |
| 4 | Ccc cgg ccc cgg ccc C | 0.327 | | | | 5321 | N/A |
| 5 | Ccc ggc ccc ggc ccc G | 0.253 | | | | 5326 | N/A |
| 6 | Cgg ccc cgg ccc cgg cC | 0.269 | | | | 5323 | N/A |
| 7 | Ccg gcc ccg gcc ccg gcC | 0.144 | | | | 5323 | N/A |
| 8 | Ccc ggc ccc ggc ccc ggc C | 0.319 | | | | 5323 | N/A |
| 9 | Gcc ccg gcc ccg gcc ccg gC | 0.202 | 4.7 | 0.9 | 0.7 | 5324 | N/A |
| 10 | GGc ccc ggc cCC | 0.083 | 5.2 | 0.1 | 7.8 | 5321 | N/A |
| 11 | GGc ccc ggc ccC G | 0.17 | 3.5 | 5.4 | 1.8 | 5326 | N/A |
| 12 | CCc cgg ccc cgg CC | 0.212 | 2.3 | 2.5 | 1.1 | 5323 | N/A |
| 13 | GCc ccg gcc ccg gCC | 0.216 | 1.1 | 1.6 | 1.2 | 5323 | N/A |
| 14 | CCc cgg ccc cgg ccC C | 0.145 | 2.2 | 1 | 4.4 | 5321 | N/A |
| 15 | CCc ggc ccc ggc ccC G | 0.17 | | | | 5326 | N/A |
| 16 | CGg ccc cgg ccc cgg CC | 0.167 | 0.9 | 0.6 | 0.1 | 5323 | N/A |
| 17 | CCg gcc ccg gcc ccg gCC | 0.238 | 1.6 | 1.5 | 1 | 5323 | N/A |
| 18 | CCc ggc ccc ggc ccc ggC C | 0.276 | | | | 5323 | N/A |
| 19 | GCc ccg gcc ccg gcc ccg GC | 0.194 | 0.3 | 0.6 | 0.8 | 5324 | N/A |
| 20 | Ggc ccc ggc ccC | 0.18 | 2.3 | 2.5 | 1.7 | 5321 | N/A |
| 21 | GGc ccc ggc cCC G | 0.144 | 7.2 | 1.9 | 2.4 | 5326 | N/A |
| 22 | GCc ccg gcc ccg GCC | 0.134 | 3.2 | 7.5 | 3.7 | 5323 | N/A |
| 23 | GCC ccg gcc ccg GCC | 0.077 | 7.7 | 5.7 | 6.2 | 5323 | N/A |
| 24 | CCc ggc ccc ggc cCC G | 0.108 | 4.3 | 9.9 | 3.6 | 5326 | N/A |
| 25 | CCC ggc ccc ggc cCC G | 0.316 | | | | 5326 | N/A |
| 26 | CCg gcc ccg gcc ccg GCC | 0.214 | 2.3 | 1 | 2.8 | 5323 | N/A |
| 27 | CCg gcc ccg gcc ccG GCC | 0.177 | | | | 5323 | N/A |
| 28 | CCG gcc ccg gcc ccg GCC | 0.189 | | | | 5323 | N/A |
| 29 | GCc ccg gcc ccg gcc ccG GC | 0.161 | | | | 5324 | N/A |
| 30 | GCC ccg gcc ccg gcc ccG GC | 0.226 | | | | 5324 | N/A |
| 31 | GGA Cac cgt agg ttA C | 0.939 | | | | 5003 | 3 |
| 32 | CTA gcg gga cac cgT A | 0.967 | | | | 5009 | 9 |
| 33 | TCT Ttc cta gcg ggA C | 0.738 | | | | 5015 | 15 |
| 34 | CAC ctc tct ttc ctA G | 0.752 | | | | 5021 | 21 |
| 35 | TTG Acg cac ctc tcT T | 0.697 | | | | 5027 | 27 |
| 36 | CGc tgt ttg acg CAC C | 0.876 | | | | 5033 | 33 |
| 37 | ACt tgt cgc tgt tTG A | 0.892 | | | | 5039 | 39 |
| 38 | GGc gga act tgt cGC T | 0.916 | | | | 5045 | 45 |
| 39 | TAC gtg ggc gga aCT T | 1.34 | | | | 5051 | 51 |
| 40 | ATC ttt tac gtg gGC G | 1.057 | | | | 5057 | 57 |
| 41 | AGC gtc atc ttt tAC G | 0.858 | | | | 5063 | 63 |
| 42 | ACA cca agc gtc aTC T | 0.865 | | | | 5069 | N/A |
| 43 | GCT gac aca cca agC G | 0.821 | | | | 5075 | N/A |
| 44 | GGG acg get gac acA C | 0.392 | | | | 5081 | N/A |
| 45 | GCa gca ggg acg gcT G | 0.499 | | | | 5087 | N/A |
| 46 | AAC cgg gca gca ggG A | 0.581 | | | | 5093 | N/A |
| 47 | AGA agc aac cgg gCA G | 0.769 | | | | 5099 | N/A |
| 48 | CAA aag aga agc aAC C | 0.621 | | | | 5105 | N/A |
| 49 | CGC Ccc caa aag agA A | 0.466 | | | | 5111 | N/A |
| 50 | AGA ccc cgc ccc caA A | 0.28 | | | | 5117 | N/A |
| 51 | Ctt get aga ccc cgC C | 0.758 | | | | 5123 | N/A |
| 52 | CCt get ctt get agA C | 0.624 | | | | 5129 | N/A |
| 53 | CCc aca cct get ctT G | 0.682 | | | | 5135 | N/A |
| 54 | CCT Aaa ccc aca ccT G | 1.124 | | | | 5141 | N/A |
| 55 | ACA Cct cct aaa ccC A | 0.968 | | | | 5147 | N/A |
| 56 | AAa cac aca cct CCT A | 1.389 | | | | 5153 | N/A |
| 57 | AGA Ggg tgg gaa aaA C | 1.396 | | | | 5171 | N/A |
| 58 | Ggg gag aga ggg tgG G | 1.521 | | | | 5177 | N/A |
| 59 | AGT agt ggg gag agA G | 0.315 | | | | 5183 | N/A |
| 60 | AGA gca agt agt ggG G | 0.466 | | | | 5189 | N/A |
| 61 | ACt gtg aga gca AGT A | 0.691 | >100 | >100 | >100 | 5195 | N/A |
| 62 | GCG agt act gtg agA G | 0.663 | | | | 5201 | N/A |
| 63 | CCc tca gcg agt acT G | 0.321 | | | | 5207 | N/A |
| 64 | TGT tca ccc tca gcG A | 0.228 | | | | 5213 | N/A |
| 65 | TTt tct tgt tca cCC T | 0.7 | | | | 5219 | N/A |
| 66 | CAG gtc ttt tct tGT T | 0.559 | | | | 5225 | N/A |
| 67 | CTT Tat cag gtc ttT T | 0.673 | | | | 5231 | N/A |
| 68 | GTt aat ctt tat CAG G | 0.519 | | | | 5237 | N/A |
| 69 | CTT Ctg gtt aat ctT T | 0.55 | | | | 5243 | N/A |
| 70 | TGT Ttt ctt ctg gtT A | 0.341 | | | | 5249 | N/A |
| 71 | CCt cct tgt ttt ctT C | 0.985 | | | | 5255 | N/A |
| 72 | TGt ttc cct cct tgT T | 0.468 | | | | 5261 | N/A |
| 73 | TGc ggt tgt ttc ccT C | 0.464 | | | | 5267 | N/A |
| 74 | ACA ggc tgc ggt tgT T | 0.315 | | | | 5273 | N/A |
| 75 | CTT get aca ggc tgC G | 0.944 | | | | 5279 | N/A |
| 76 | CCA gag ctt get acA G | 0.287 | | | | 5285 | N/A |
| 77 | TGa gtt cca gag cTT G | 0.371 | | | | 5291 | N/A |
| 78 | GAc tcc tga gtt cCA G | 0.46 | | | | 5297 | N/A |
| 79 | GCg cgc gac tcc tgA G | 0.228 | | | | 5303 | N/A |
| 80 | CCc cta gcg cgc gaC T | 0.431 | | | | 5309 | N/A |
| 81 | CCG gcc ccg GCC | 0.828 | | | | 5323 | N/A |
| 82 | CCg gcc ccG GCC | 0.902 | | | | 5323 | N/A |
| 83 | CCg gcc ccg GCC | 0.392 | | | | 5323 | N/A |
| 84 | CCC ggc ccc GGC | 0.608 | | | | 5324 | N/A |
| 85 | CCc ggc ccC GGC | 0.408 | | | | 5324 | N/A |
| 86 | CCc ggc ccc GGC | 0.217 | | | | 5324 | N/A |
| 87 | CCC cgg ccc CGG | 0.356 | | | | 5325 | N/A |
| 88 | CCc cgg ccC CGG | 0.494 | | | | 5325 | N/A |
| 89 | CCc cgg ccc CGG | 0.426 | | | | 5325 | N/A |
| 90 | CGG ccc cgg cCC C | 0.31 | | | | 5321 | N/A |
| 91 | CGg ccc cgg CCC C | 0.444 | | | | 5321 | N/A |
| 92 | CGg ccc cgg cCC C | 0.224 | | | | 5321 | N/A |
| 93 | CCG gcc ccg gCC C | 0.698 | | | | 5322 | N/A |
| 94 | CCg gcc ccg GCC C | 0.586 | | | | 5322 | N/A |
| 95 | CCg gcc ccg gCC C | 0.144 | 13.7 | 4.6 | 15.8 | 5322 | N/A |
| 96 | GCC ccg gcc cCG G | 0.332 | | | | 5325 | N/A |
| 97 | GCc ccg gcc CCG G | 0.709 | | | | 5325 | N/A |
| 98 | GCc ccg gcc cCG G | 0.13 | 3.2 | 5.4 | 9.5 | 5325 | N/A |
| 99 | GGC ccc ggc cCC G | 0.429 | | | | 5326 | N/A |
| 100 | GGc ccc ggc CCC G | 0.386 | | | | 5326 | N/A |
| 101 | CCG gcc ccg gcC CC | 0.306 | | | | 5321 | N/A |
| 102 | CCg gcc ccg gCC CC | 0.489 | | | | 5321 | N/A |
| 103 | CCg gcc ccg gcC CC | 0.126 | 4.7 | 3 | 0.8 | 5321 | N/A |
| 104 | CCC ggc ccc ggC CC | 0.476 | | | | 5322 | N/A |
| 105 | CCc ggc ccc gGC CC | 0.504 | | | | 5322 | N/A |
| 106 | CCc ggc ccc ggC CC | 0.192 | 30 | 1.9 | 0.4 | 5322 | N/A |
| 107 | GGC ccc ggc ccC GG | 0.467 | | | | 5325 | N/A |
| 108 | GGc ccc ggc cCC GG | 0.54 | | | | 5325 | N/A |
| 109 | GGc ccc ggc ccC GG | 0.164 | 3.6 | 2.4 | 0.8 | 5325 | N/A |
| 110 | CGG ccc cgg ccC CG | 0.245 | | | | 5326 | N/A |
| 111 | CGg ccc cgg cCC CG | 0.124 | | | | 5326 | N/A |
| 112 | CGg ccc cgg ccC CG | 0.113 | | | | 5326 | N/A |
| 113 | CCC ggc ccc ggc CCC | 0.335 | | | | 5321 | N/A |
| 114 | CCc ggc ccc ggC CCC | 0.327 | | | | 5321 | N/A |
| 115 | CCc ggc ccc ggc CCC | 0.23 | | | | 5321 | N/A |
| 116 | GCc ccg gcc ccG GCC | 0.246 | | | | 5323 | N/A |
| 117 | CGG ccc cgg ccc CGG | 0.213 | | | | 5325 | N/A |
| 118 | CGg ccc cgg ccC CGG | 0.452 | | | | 5325 | N/A |
| 119 | CGg ccc cgg ccc CGG | 0.161 | 2.8 | 2.2 | 0.7 | 5325 | N/A |
| 120 | CCG gcc ccg gcc CCG | 0.305 | 92.1 | 17.4 | 16.7 | 5326 | N/A |
| 121 | CCg gcc ccg gcC CCG | 0.389 | | | | 5326 | N/A |
| 122 | CCg gcc ccg gcc CCG | 0.202 | | | | 5326 | N/A |
| 123 | CCC cgg ccc cgg cCC C | 0.292 | 14 | 16.7 | 1.6 | 5321 | N/A |
| 124 | CCc cgg ccc cgg CCC C | 0.137 | | | | 5321 | N/A |
| 125 | CCc cgg ccc cgg cCC C | 0.261 | 32.6 | 1.3 | 7.2 | 5321 | N/A |
| 126 | CGG ccc cgg ccc cGG C | 0.264 | 2.6 | 2 | 0.7 | 5324 | N/A |
| 127 | CGg ccc cgg ccc CGG C | 0.148 | 8.1 | 0.5 | 2.4 | 5324 | N/A |
| 128 | CGg ccc cgg ccc cGG C | 0.175 | | | | 5324 | N/A |
| 129 | CCG gcc ccg gcc cCG G | 0.464 | | | | 5325 | N/A |
| 130 | CCg gcc ccg gcc CCG G | 0.482 | | | | 5325 | N/A |
| 131 | CCg gcc ccg gcc cCG G | 0.604 | | | | 5325 | N/A |
| 132 | CCc ggc ccc ggc CCC G | 0.336 | | | | 5326 | N/A |
| 133 | CGG ccc cgg ccc cgG CC | 0.219 | | | | 5323 | N/A |
| 134 | CGg ccc cgg ccc cGG CC | 0.218 | 22 | 11.1 | 10.2 | 5323 | N/A |
| 135 | CGg ccc cgg ccc cgG CC | 0.138 | 16.6 | 0.5 | 0.8 | 5323 | N/A |
| 136 | CCG gcc ccg gcc ccG GC | 0.235 | | | | 5324 | N/A |
| 137 | CCg gcc ccg gcc cCG GC | 0.293 | | | | 5324 | N/A |
| 138 | CCg gcc ccg gcc ccG GC | 0.345 | | | | 5324 | N/A |
| 139 | CCC ggc ccc ggc ccC GG | 0.407 | | | | 5325 | N/A |
| 140 | CCc ggc ccc ggc cCC GG | 0.334 | | | | 5325 | N/A |
| 141 | CCc ggc ccc ggc ccC GG | 0.245 | | | | 5325 | N/A |
| 142 | CCC cgg ccc cgg ccC CG | 0.197 | 6.6 | 1.4 | 1.4 | 5326 | N/A |
| 143 | CCc cgg ccc cgg cCC CG | 0.192 | | | | 5326 | N/A |
| 144 | CCc cgg ccc cgg ccC CG | 0.16 | 70.6 | 3 | 1.8 | 5326 | N/A |
| 145 | CGG ccc cgg ccc cgg CCC | 0.176 | 3.7 | 10.7 | 5.9 | 5322 | N/A |
| 146 | CGg ccc cgg ccc cgG CCC | 0.144 | 0.7 | 3.7 | 1 | 5322 | N/A |
| 147 | CGg ccc cgg ccc cgg CCC | 0.009 | 2.3 | 1.4 | 1.1 | 5322 | N/A |
| 148 | CCC ggc ccc ggc ccc GGC | 0.253 | | | | 5324 | N/A |
| 149 | CCc ggc ccc ggc ccC GGC | 0.288 | 6 | 4.6 | 1.3 | 5324 | N/A |
| 150 | CCc ggc ccc ggc ccc GGC | 0.237 | | | | 5324 | N/A |
| 151 | CCC cgg ccc cgg ccc CGG | 0.147 | >100 | 5.8 | 4.7 | 5325 | N/A |
| 152 | CCc cgg ccc cgg ccC CGG | 0.107 | 8 | 17.1 | 3.2 | 5325 | N/A |
| 153 | CCc cgg ccc cgg ccc CGG | 0.113 | 3.4 | 5.8 | 0.5 | 5325 | N/A |
| 154 | CGG ccc cgg ccc cgg cCC C | 0.111 | 1.8 | 0.8 | 0.5 | 5321 | N/A |
| 155 | CGg ccc cgg ccc cgg CCC C | 0.119 | 2.2 | 4.1 | 1 | 5321 | N/A |
| 156 | CGg ccc cgg ccc cgg cCC C | 0.207 | 0.6 | 0.5 | 0.2 | 5321 | N/A |
| 157 | CCG gcc ccg gcc ccg gCC C | 0.072 | 9.8 | 0.5 | 0.2 | 5322 | N/A |
| 158 | CCg gcc ccg gcc ccg GCC C | 0.182 | 1.3 | 0.7 | 0.5 | 5322 | N/A |
| 159 | CCg gcc ccg gcc ccg gCC C | 0.094 | 2.3 | 1.8 | 0.8 | 5322 | N/A |
| 160 | CCC ggc ccc ggc ccc gGC C | 0.108 | 8.8 | 3.5 | 1.6 | 5323 | N/A |
| 161 | CCc ggc ccc ggc ccc GGC C | 0.113 | 5.7 | 2.4 | 0.3 | 5323 | N/A |
| 162 | CCc ggc ccc ggc ccc gGC C | 0.106 | 2.8 | 1 | 0.8 | 5323 | N/A |
| 163 | GGC ccc ggc ccc ggc cCC G | 0.099 | 2.4 | 0.6 | 0.4 | N/A | N/A |
| 164 | GGc ccc ggc ccc ggc CCC G | 0.253 | | | | N/A | N/A |
| 165 | GGc ccc ggc ccc ggc cCC G | 0.171 | | | | N/A | N/A |
| 166 | CCG gcc ccg gcc ccg gcC CC | 0.055 | 4.2 | 0.3 | 4 | 5321 | N/A |
| 167 | CCg gcc ccg gcc ccg gCC CC | 0.14 | 7.5 | 7.9 | 3.5 | 5321 | N/A |
| 168 | CCg gcc ccg gcc ccg gcC CC | 0.17 | | | | 5321 | N/A |
| 169 | CCC cgg ccc cgg ccc cgG CC | 0.176 | 4.5 | 6.2 | 0.2 | 5323 | N/A |
| 170 | CCc cgg ccc cgg ccc cGG CC | 0.24 | 3 | 3.3 | 1.4 | 5323 | N/A |
| 171 | CCc cgg ccc cgg ccc cgG CC | 0.322 | | | | 5323 | N/A |
| 172 | GCc ccg gcc ccg gcc cCG GC | 0.211 | 6.1 | 9 | 0.5 | 5324 | N/A |
| 173 | CGG ccc cgg ccc cgg ccC CG | 0.114 | 3.5 | 0.7 | 0.4 | N/A | N/A |
| 174 | CGg ccc cgg ccc cgg cCC CG | 0.17 | 4.7 | 19.3 | 12.6 | N/A | N/A |
| 175 | CGg ccc cgg ccc cgg ccC CG | 0.156 | 2.4 | 0.4 | 1.1 | N/A | N/A |
| 176 | CGT cag tat gcg AAT c | 0.9 | >100 | | | N/A | N/A |

| Oligo- | | | | | |
|---|---|---|---|---|---|
| mer No. | Total | Gap | | 5′ | 3′ |
| (SEQ ID | 5mC | 5mC | LNA | Wing | Wing |
| NO:) | Count | Count | Count | Length | Length |
| 1 | 2 | 2 | 2 | 1 | 1 |
| 2 | 7 | 2 | 2 | 1 | 1 |
| 3 | 3 | 2 | 2 | 1 | 1 |
| 4 | 4 | 2 | 2 | 1 | 1 |
| 5 | 3 | 3 | 2 | 1 | 1 |
| 6 | 5 | 2 | 2 | 1 | 1 |
| 7 | 6 | 3 | 2 | 1 | 1 |
| 8 | 4 | 3 | 2 | 1 | 1 |
| 9 | 2 | 3 | 2 | 1 | 1 |
| 10 | 4 | 1 | 4 | 2 | 2 |
| 11 | 3 | 1 | 4 | 2 | 2 |
| 12 | 0 | 2 | 4 | 2 | 2 |
| 13 | 3 | 2 | 4 | 2 | 2 |
| 14 | 5 | 2 | 4 | 2 | 2 |
| 15 | 3 | 2 | 4 | 2 | 2 |
| 16 | 2 | 2 | 4 | 2 | 2 |
| 17 | 2 | 2 | 4 | 2 | 2 |
| 18 | 5 | 3 | 4 | 2 | 2 |
| 19 | 2 | 3 | 4 | 2 | 2 |
| 20 | 2 | 1 | 2 | 1 | 1 |
| 21 | 6 | 1 | 5 | 2 | 3 |
| 22 | 3 | 2 | 5 | 2 | 3 |
| 23 | 4 | 2 | 6 | 3 | 3 |
| 24 | 3 | 2 | 5 | 2 | 3 |
| 25 | 4 | 1 | 6 | 3 | 3 |
| 26 | 1 | 2 | 5 | 2 | 3 |
| 27 | 2 | 2 | 6 | 2 | 4 |
| 28 | 1 | 2 | 6 | 3 | 3 |
| 29 | 2 | 3 | 5 | 2 | 3 |
| 30 | 1 | 3 | 6 | 3 | 3 |
| 31 | 7 | 1 | 6 | 4 | 2 |
| 32 | 7 | 2 | 5 | 3 | 2 |
| 33 | 5 | 1 | 6 | 4 | 2 |
| 34 | 6 | 0 | 5 | 3 | 2 |
| 35 | 6 | 1 | 6 | 4 | 2 |
| 36 | 7 | 1 | 6 | 2 | 4 |
| 37 | 7 | 1 | 5 | 2 | 3 |
| 38 | 4 | 2 | 5 | 2 | 3 |
| 39 | 5 | 1 | 6 | 3 | 3 |
| 40 | 7 | 1 | 6 | 3 | 3 |
| 41 | 7 | 0 | 6 | 3 | 3 |
| 42 | 7 | 1 | 6 | 3 | 3 |
| 43 | 7 | 0 | 5 | 3 | 2 |
| 44 | 7 | 1 | 5 | 3 | 2 |
| 45 | 5 | 1 | 4 | 2 | 2 |
| 46 | 5 | 1 | 5 | 3 | 2 |
| 47 | 6 | 1 | 6 | 3 | 3 |
| 48 | 7 | 0 | 6 | 3 | 3 |
| 49 | 7 | 0 | 6 | 4 | 2 |
| 50 | 5 | 1 | 5 | 3 | 2 |
| 51 | 7 | 1 | 3 | 1 | 2 |
| 52 | 6 | 0 | 4 | 2 | 2 |
| 53 | 6 | 0 | 4 | 2 | 2 |
| 54 | 5 | 0 | 6 | 4 | 2 |
| 55 | 8 | 0 | 6 | 4 | 2 |
| 56 | 6 | 0 | 6 | 2 | 4 |
| 57 | 5 | 0 | 6 | 4 | 2 |
| 58 | 2 | 0 | 3 | 1 | 2 |
| 59 | 6 | 0 | 5 | 3 | 2 |
| 60 | 4 | 0 | 5 | 3 | 2 |
| 61 | 6 | 0 | 6 | 2 | 4 |
| 62 | 6 | 0 | 5 | 3 | 2 |
| 63 | 3 | 1 | 4 | 2 | 2 |
| 64 | 2 | 1 | 5 | 3 | 2 |
| 65 | 6 | 0 | 5 | 2 | 3 |
| 66 | 5 | 0 | 6 | 3 | 3 |
| 67 | 6 | 0 | 6 | 4 | 2 |
| 68 | 5 | 0 | 6 | 2 | 4 |
| 69 | 5 | 0 | 6 | 4 | 2 |
| 70 | 3 | 0 | 6 | 4 | 2 |
| 71 | 7 | 0 | 4 | 2 | 2 |
| 72 | 4 | 0 | 4 | 2 | 2 |
| 73 | 8 | 1 | 4 | 2 | 2 |
| 74 | 6 | 1 | 5 | 3 | 2 |
| 75 | 8 | 0 | 5 | 3 | 2 |
| 76 | 5 | 0 | 5 | 3 | 2 |
| 77 | 4 | 0 | 5 | 2 | 3 |
| 78 | 5 | 0 | 5 | 2 | 3 |
| 79 | 0 | 2 | 4 | 2 | 2 |
| 80 | 3 | 3 | 4 | 2 | 2 |
| 81 | 7 | 1 | 6 | 3 | 3 |
| 82 | 5 | 1 | 6 | 2 | 4 |
| 83 | 8 | 1 | 5 | 2 | 3 |
| 84 | 7 | 1 | 6 | 3 | 3 |
| 85 | 5 | 1 | 6 | 2 | 4 |
| 86 | 1 | 2 | 5 | 2 | 3 |
| 87 | 2 | 1 | 6 | 3 | 3 |
| 88 | 7 | 1 | 6 | 2 | 4 |
| 89 | 3 | 1 | 5 | 2 | 3 |
| 90 | 6 | 1 | 6 | 3 | 3 |
| 91 | 5 | 1 | 6 | 2 | 4 |
| 92 | 1 | 1 | 5 | 2 | 3 |
| 93 | 6 | 1 | 6 | 3 | 3 |
| 94 | 6 | 1 | 6 | 2 | 4 |
| 95 | 6 | 1 | 5 | 2 | 3 |
| 96 | 6 | 1 | 6 | 3 | 3 |
| 97 | 6 | 1 | 6 | 2 | 4 |
| 98 | 2 | 1 | 5 | 2 | 3 |
| 99 | 4 | 1 | 6 | 3 | 3 |
| 100 | 5 | 1 | 6 | 2 | 4 |
| 101 | 5 | 1 | 6 | 3 | 3 |
| 102 | 4 | 1 | 6 | 2 | 4 |
| 103 | 5 | 1 | 5 | 2 | 3 |
| 104 | 4 | 1 | 6 | 3 | 3 |
| 105 | 5 | 2 | 6 | 2 | 4 |
| 106 | 4 | 2 | 5 | 2 | 3 |
| 107 | 4 | 1 | 6 | 3 | 3 |
| 108 | 5 | 1 | 6 | 2 | 4 |
| 109 | 5 | 1 | 5 | 2 | 3 |
| 110 | 2 | 1 | 6 | 3 | 3 |
| 111 | 7 | 1 | 6 | 2 | 4 |
| 112 | 5 | 1 | 5 | 2 | 3 |
| 113 | 7 | 1 | 6 | 3 | 3 |
| 114 | 6 | 2 | 6 | 2 | 4 |
| 115 | 3 | 2 | 5 | 2 | 3 |
| 116 | 1 | 2 | 6 | 2 | 4 |
| 117 | 1 | 1 | 6 | 3 | 3 |
| 118 | 6 | 1 | 6 | 2 | 4 |
| 119 | 1 | 1 | 5 | 2 | 3 |
| 120 | 6 | 1 | 6 | 3 | 3 |
| 121 | 4 | 1 | 6 | 2 | 4 |
| 122 | 1 | 1 | 5 | 2 | 3 |
| 123 | 4 | 2 | 6 | 3 | 3 |
| 124 | 5 | 2 | 6 | 2 | 4 |
| 125 | 5 | 2 | 5 | 2 | 3 |
| 126 | 5 | 2 | 6 | 3 | 3 |
| 127 | 3 | 1 | 6 | 2 | 4 |
| 128 | 2 | 2 | 5 | 2 | 3 |
| 129 | 8 | 1 | 6 | 3 | 3 |
| 130 | 5 | 1 | 6 | 2 | 4 |
| 131 | 5 | 1 | 5 | 2 | 3 |
| 132 | 7 | 2 | 6 | 2 | 4 |
| 133 | 1 | 2 | 6 | 3 | 3 |
| 134 | 2 | 2 | 6 | 2 | 4 |
| 135 | 6 | 2 | 5 | 2 | 3 |
| 136 | 0 | 2 | 6 | 3 | 3 |
| 137 | 5 | 1 | 6 | 2 | 4 |
| 138 | 3 | 2 | 5 | 2 | 3 |
| 139 | 7 | 1 | 6 | 3 | 3 |
| 140 | 7 | 2 | 6 | 2 | 4 |
| 141 | 0 | 2 | 5 | 2 | 3 |
| 142 | 3 | 2 | 6 | 3 | 3 |
| 143 | 3 | 2 | 6 | 2 | 4 |
| 144 | 3 | 2 | 5 | 2 | 3 |
| 145 | 2 | 2 | 6 | 3 | 3 |
| 146 | 6 | 2 | 6 | 2 | 4 |
| 147 | 2 | 2 | 5 | 2 | 3 |
| 148 | 6 | 2 | 6 | 3 | 3 |
| 149 | 5 | 2 | 6 | 2 | 4 |
| 150 | 2 | 3 | 5 | 2 | 3 |
| 151 | 6 | 2 | 6 | 3 | 3 |
| 152 | 4 | 2 | 6 | 2 | 4 |
| 153 | 6 | 2 | 5 | 2 | 3 |
| 154 | 6 | 2 | 6 | 3 | 3 |
| 155 | 6 | 2 | 6 | 2 | 4 |
| 156 | 0 | 2 | 5 | 2 | 3 |
| 157 | 3 | 2 | 6 | 3 | 3 |
| 158 | 3 | 2 | 6 | 2 | 4 |
| 159 | 4 | 2 | 5 | 2 | 3 |
| 160 | 2 | 2 | 6 | 3 | 3 |
| 161 | 5 | 3 | 6 | 2 | 4 |
| 162 | 5 | 3 | 5 | 2 | 3 |
| 163 | 5 | 2 | 6 | 3 | 3 |
| 164 | 5 | 2 | 6 | 2 | 4 |
| 165 | 3 | 2 | 5 | 2 | 3 |
| 166 | 4 | 2 | 6 | 3 | 3 |
| 167 | 6 | 2 | 6 | 2 | 4 |
| 168 | 3 | 2 | 5 | 2 | 3 |
| 169 | 2 | 3 | 6 | 3 | 3 |
| 170 | 2 | 3 | 6 | 2 | 4 |
| 171 | 4 | 3 | 5 | 2 | 3 |
| 172 | 0 | 2 | 6 | 2 | 4 |
| 173 | 5 | 2 | 6 | 3 | 3 |
| 174 | 2 | 2 | 6 | 2 | 4 |
| 175 | 3 | 2 | 5 | 2 | 3 |
| 176 | 5 | 1 | 6 | 3 | 3 |

Column 1 (from left) lists the identification number assigned to each specific oligomer; these identification numbers also correspond to the sequence identification number (SEQ ID NO). Column 2 identifies the nucleobase sequence of each oligomer, including the bases in the 5′ flanking region, the gap region and the 3′ flanking region. In each oligomer, the 5′ flanking region and 3′ flanking region each consist of one or more beta-D-oxy-LNA monomers identified by capital letters (i.e., A, T, G, C), whereas the gap region consists of DNA monomers identified by lower case letters (i.e., a, t, g, c). All internucleoside linkages are phosphorothioate. Additionally, certain oligomers described in Table 4 contain cytosine bases modified to be 5-methylcytosine. Specifically, all LNA cytosines in 5′ flanking regions and 3′ flanking regions (i.e., those denoted by capital “C”) are 5-methylcytosine. Furthermore, in the DNA gap regions, any cytosine that immediately precedes a guanine (i.e., those denoted by “cg” or “cG”), are 5-methylcytosine. Other cytosines in gap regions are unmodified. Column 3 reports the maximum C9ORF72 RNA reduction effected using 25 μM of each oligomer tested in the primary screen. Where tested, Columns 4, 5, and 6 report IC₅₀ values for the oligomers. Specifically, Column 4 reports the concentration in μM of tested oligomers sufficient to reduce C9ORF72 total pre-mRNA by 50% (qper-assay#1); Column 5 reports the concentration in μM of tested oligomers sufficient to reduce C9ORF72 sense pre-mRNA by 50% (qper-assay#2); and Column 6 reports the concentration in μM of tested oligomers sufficient to reduce C9ORF72 antisense pre-mRNA (qper-assay#2). Column 7 indicates the location on the reference genomic DNA (SEQ ID NO:187) to which the oligomer is complementary. Column 8 indicates the location on the reference mRNA (SEQ ID NO:188) to which the oligomer is complementary, if applicable. Columns 9 and 10 provide the number of 5-methylcytosines within the oligomer: Column 9 provides the total number of 5-methylcytosines in the oligomer, and Column 10 provides the number of 5-methylcytosines within the gap region. Column 11 provides the total number of locked nucleic acids (LNAs) within the oligomer. Columns 11 and 12 provide the lengths of the 5′ and 3′ flanking regions within each gapmer (“wings”), respectively.

### Example 4

### Comparative Inhibition of C9ORF72 Sense and Antisense Transcripts by MOE- and LNA-Based Gapmers

Oligomer 109 is an LNA-containing gapmer consisting of the sequence of SEQ ID NO: 109. The efficacy of this oligomer was compared to a number of gapmers comprising 2′-MOE chemistry, as shown in Table 5. Column 1 lists the sequence identification numbers for the 2′-MOE gapmers used in this Example. Column 2 provides the lengths of the oligomers. Column 3 provides the chemistry and the length of the 5′ flanking region (wing), gap region and 3′ flanking region (wing), respectively. For example, a 3-10-3 gapmer is 16 nucleosides in length, wherein the central gap segment comprises ten 2′-deoxynucleosides and is flanked by wing segments on both the 5′ end and on the 3′ end comprising three nucleosides each. Column 4 provides the sequence and structure of the gapmer. Each nucleoside in the 5′ wing segment and each nucleoside in the 3′ wing segment comprises a 2′-MOE group.

TABLE 5

| | SEQ | | Chem- | |
|---|---|---|---|---|
| Oligo | ID | | istry | |
| Name | NO: | Length | Structure | Sequence |
| MOE1 | 212 | 16 | 2′-MOE | GGC ccc ggc |
| | | | 3-10-3 | ccc gGC C |
| MOE2 | 196 | 20 | 2′-MOE | CCG GCc ccg gcc |
| | | | 5-10-5 | ccg GCC CC |
| MOE3 | 234 | 19 | 2′-MOE | GGC CCc ggc ccc |
| | | | 5-10-4 | ggc CCC G |
| MOE4 | 235 | 20 | 2′-MOE | GCC TTa ctc tag |
| | | | 5-10-5 | gac CAA GA |

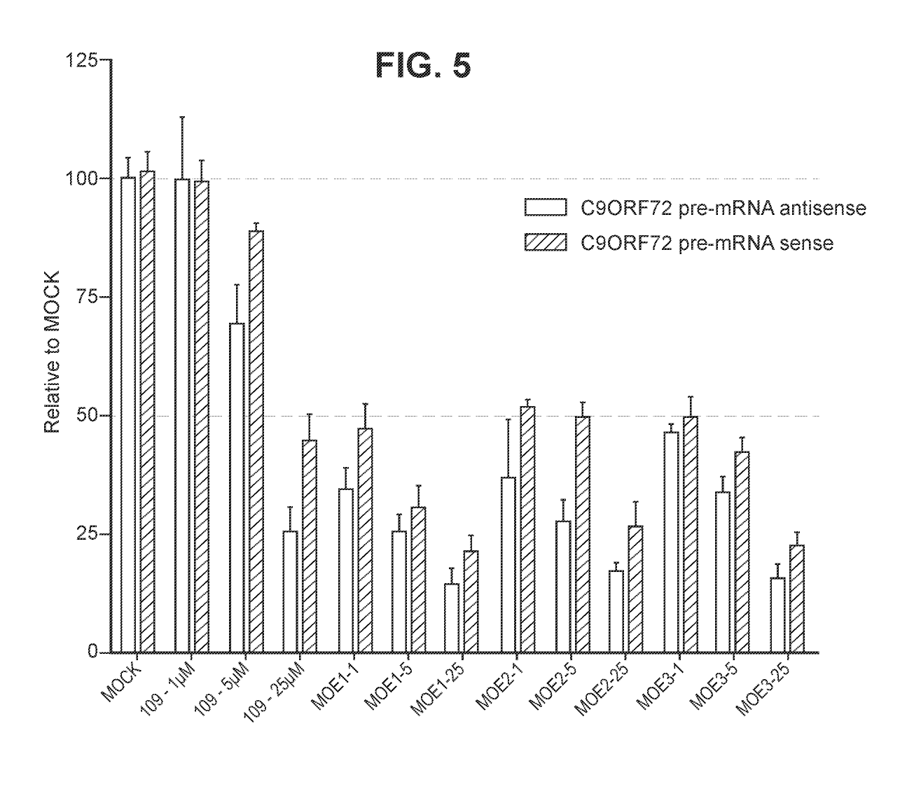

The LNA-based oligomer No. 109 and the 2′-MOE-based oligos listed in Table 5 were tested for the ability to inhibit expression of C9ORF72 sense and antisense transcripts. The methods used for this example are essentially as described above, for example in Example 2. Briefly, ND40063 fibroblasts were treated with the respective oligonucleotides by gymnotic delivery at 1 μM, 5 μM and 25 μM, and incubated for 72 hours prior to RNA isolation and analysis as described above. Sense and antisense transcript levels for each treatment were measured and normalized to RNA from mock transfected cells. As shown in FIG. 5, the LNA-based Oligomer No. 109 was equally effective in inhibiting expression of both C9ORF72 sense and antisense transcripts as several of the 2′-MOE-based oligomers tested.

### SPECIFIC EMBODIMENTS, CITATION OF REFERENCES

The present disclosure is not to be limited in scope by the specific embodiments described herein. Indeed, various modifications of the disclosure in addition to those described herein will become apparent to those skilled in the art from the foregoing description and accompanying figures. Such modifications are intended to fall within the scope of the appended claims.

Various references, including patent applications, patents, and scientific publications, are cited herein; the disclosure of each such reference is hereby incorporated herein by reference in its entirety.

What is claimed is:

1. A gapmer comprising between 12 to 30 nucleosides, complementary to at least a 12 contiguous nucleobase portion of SEQ ID NO:187, wherein the gapmer further comprises in 5′ to 3′ order:
(a) a 5′ flanking region consisting of 1 to 5 contiguously linked nucleosides, at least one of which is an LNA monomer,
(b) a gap region consisting of contiguously linked deoxyribonucleosides; and
(c) a 3′ flanking region consisting of 1 to 5 contiguously linked nucleosides, at least one of which is an LNA monomer.
wherein the gapmer is capable of preferentially inhibiting expression of C9ORF72 sense transcript containing an expanded hexanucleotide repeat region when compared with inhibiting expression of a wild-type C9ORF72 sense transcript, and
wherein the gapmer is further capable of inhibiting expression of C9ORF72 antisense transcript containing an expanded hexanucleotide repeat region.

2. The gapmer of claim 1, having a sequence that is identical to at least a 12 nucleobase portion of a nucleobase sequence selected from the group consisting of SEQ ID NO:1 to SEQ ID NO:175.

3. The gapmer of any of the preceding claims, wherein all internucleoside linkages are phosphorothioate.

4. The gapmer of claim 1, wherein all of the nucleosides in the 5′ flanking region and 3′ flanking region are modified.

5. The gapmer of claim 1, wherein all modified nucleosides are LNA monomers.

6. The gapmer of claim 1, wherein all cytosine nucleosides in the 5′ flanking region and 3′ flanking region are 5-methylcytosines.

7. The gapmer of claim 1, wherein the nucleobase sequence of said gapmer is GGc ccc ggc ccC GG (SEQ ID NO:109), wherein the 5′ flanking region and 3′ flanking region are each respectively indicated by capital letters, and wherein the gap region is indicated by lower case letters.

8. The gapmer of claim 7, wherein cytosines at positions 6 and 12 are 5-methylcytosines.

9. The gapmer of claim 8, wherein all the nucleosides in the 5′ flanking region and 3′ flanking region are LAN monomers.

10. A gapmer consisting of 14 nucleotides wherein the nucleobase sequence consists of the sequence of SEQ ID NO:109, and the 5′ flanking region consists of two nucleosides that are LNA monomers, the 3′ flanking region consists of three nucleosides that are LNA monomers, wherein all internucleoside linkages are phosphorothioate linkages, and each cytosine at positions 6 and 12 is a 5-methylcytosine.

11. A composition comprising the gapmer of any of the preceding claims and a pharmaceutically acceptable carrier.

12. A method of treating a disorder in a subject in need thereof wherein the subject is suffering from a disease or disorder mediated by or associated with repeat expansion in a C9ORF72 sequence, comprising administering to said subject a therapeutically effective amount of the gapmer of any of the preceding claims.

13. The method of any of claim 12, wherein said neurological disorder is selected from amyotrophic lateral sclerosis (ALS) and frontotemporal dementia (FTD).

14. The method of any of claim 13, wherein the gapmer is administered into the central nervous system intrathecally or intraventricularly.

15. A method of reducing the amount of RNA in a cell from a C9ORF72 gene containing an expanded hexanucleotide repeat region, the method comprising contacting a cell containing C9ORF72 RNA with an effective amount of the gapmer of claim 1, thereby reducing the amount of C9ORF72 RNA in the cell.

16. The method of claim 15, wherein the RNA is a C9ORF72 sense transcript.

17. The method of any of claim 15, wherein the RNA is a C9ORF72 antisense transcript.

18. A method of simultaneously down-regulating the expression of C9ORF72 sense transcript containing an expanded hexanucleotide repeat region and C9ORF72 antisense transcript containing an expanded hexanucleotide repeat region in a cell, tissue or organism comprising contacting or administering said cell, tissue or organism with an effective amount of one or more of the gapmers of claim 1.
